(19) United States
(12) Patent Application Publication
(10) Pub. No.: US 2025/0018068 A1
(43) Pub. Date: Jan. 16, 2025

(54) 4H-IMIDAZO[1,5-B]PYRAZOLE DERIVATIVES FOR DIAGNOSIS

(71) Applicant: AC IMMUNE SA, Lausanne (CH)

(72) Inventor: Jérôme Molette, Prevessin-Moens (FR)

(21) Appl. No.: 18/709,187

(22) PCT Filed: Nov. 10, 2022
(86) PCT No.: PCT/EP2022/081555

§ 371 Date: May 10, 2024

(30) Foreign Application Priority Data
Nov. 10, 2021 (EP) 21207613.7

(51) Int. Cl. A61K 51/04; C07D 487/04

(52) U.S. Cl. CPC A61K 51/0455; C07D 487/04

(57) ABSTRACT

The present invention relates to novel compounds of formula (I), or a detectably labelled compound, stereoisomer, racemic mixture, pharmaceutically acceptable salt, hydrate, or solvate thereof, that can be employed in the imaging of alpha-synuclein aggregates and determining an amount thereof. Furthermore, the compounds can be used for diagnosing a disease, disorder or abnormality associated with an alpha-synuclein aggregates, including, but not limited to, Lewy bodies and/or Lewy neurites (such as Parkinson's disease), determining a predisposition to such a disease, disorder or abnormality, prognosing such a disease, disorder or abnormality, monitoring the evolution of the disease in a patient suffering from such a disease, disorder or abnormality, monitoring the progression of such a disease, disorder or abnormality and predicting responsiveness of a patient suffering from such a disease, disorder or abnormality to a treatment thereof.

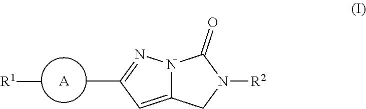

### FIELD OF THE INVENTION

The present invention relates to novel compounds of formula (I), or a detectably labelled compound, stereoisomer, racemic mixture, pharmaceutically acceptable salt, hydrate, or solvate thereof, that can be employed in the imaging of alpha-synuclein aggregates and determining an amount thereof. Furthermore, the compounds can be used for diagnosing a disease, disorder or abnormality associated with alpha-synuclein (α-synuclein, A-synuclein, aSynuclein, A-syn, α-syn, aSyn, a-syn) aggregates, including, but not limited to, Lewy bodies and/or Lewy neurites (such as Parkinson's disease), determining a predisposition to such a disease, disorder or abnormality, prognosing such a disease, disorder or abnormality, monitoring the evolution of the disease in a patient suffering from such a disease, disorder or abnormality, monitoring the progression of such a disease, disorder or abnormality and predicting responsiveness of a patient suffering from such a disease, disorder or abnormality to a treatment thereof. The present invention also relates to processes for the preparation of the compounds and their precursors, diagnostic compositions comprising the compounds, methods of using the compounds, kits comprising the compounds and their uses thereof.

### BACKGROUND OF THE INVENTION

Many diseases of aging are based on or associated with extracellular or intracellular deposits of amyloid or amyloid-like proteins that contribute to the pathogenesis as well as to the progression of the disease. The best characterized amyloid protein that forms extracellular aggregates is amyloid beta (Abeta or Aβ).

Amyloid-like proteins that form mainly intracellular aggregates include, but are not limited to, Tau, alpha-synuclein, and huntingtin (HTT). Diseases involving alpha-synuclein aggregates are generally listed as synucleinopathies (or alpha-synucleinopathies) and these include, but are not limited to, Parkinson's disease (PD). Synucleinopathies with primarily neuronal aggregates include, but are not limited to, Parkinson's disease (sporadic, familial with SNCA (the gene encoding for the alpha-synuclein protein) mutations or SNCA gene duplication or triplication, familial with mutations in other genes than SNCA, pure autonomic failure and Lewy body dysphagia), SNCA duplication carrier, Lewy Body dementia (LBD), dementia with Lewy bodies (DLB) (“pure” Lewy body dementia), Parkinson's disease dementia (PDD), diffuse Lewy body disease (DLBD), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease and normal aging in Down syndrome. Synucleinopathies with neuronal and glial aggregates of alpha-synuclein include, but are not limited to, multiple system atrophy (MSA) (Shy-Drager syndrome, striatonigral degeneration and olivopontocerebellar atrophy). Other diseases that may have alpha-synuclein-immunoreactive lesions are, but are not limited to, traumatic brain injury, chronic traumatic encephalopathy, dementia puglistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder (Jellinger, Mov. Disord. 2003, 18 Suppl. 6, S2-12; Galvin et al. JAMA Neurology 2001, 58 (2), 186-190; Kovari et al., Acta Neuropathol. 2007, 114(3), 295-8; Saito et al., J. Neuropathol. Exp. Neurol. 2004, 63(4), 323-328; McKee et al., Brain, 2013, 136 (Pt 1), 43-64; Puschmann et al., Parkinsonism Relat. Disord. 2012, 18S1, S24-S27; Usenovic et al., J. Neurosci. 2012, 32(12), 4240-4246; Winder-Rhodes et al., Mov. Disord. 2012, 27(2), 312-315; Ferman et al., J. Int. Neuropsychol. Soc. 2002, 8(7), 907-914; Smith et al., J. Pathol. 2014; 232:509-521, Lippa et al., Ann Neurol. 1999 March; 45(3):353-7; Schmitz et al., Mol. Neurobiol. 2018 Aug. 22; Charles et al., Neurosci. Lett. 2000 Jul. 28; 289(1):29-32; Wilhelmsen et al., Arch Neurol. 2004 March; 61(3):398-406; Yamaguchi et al., J. Neuropathol. Exp. Neurol. 2004, 80th annual meeting, vol. 63; Askanas et al., J. Neuropathol. Exp. Neurol. 2000 July; 59(7):592-8).

Alpha-synuclein is a 140 amino acid natively unfolded protein (Iwai et al., Biochemistry 1995, 34(32), 10139-10145). The sequence of alpha-synuclein can be divided into three main domains: 1) the N-terminal region comprising of residues 1-60, which contains the 11-mer amphipatic imperfect repeat residues with highly conserved hexamer (KTKEGV). This region has been implicated in regulating alpha-synuclein binding to membranes and its internalization; 2) the hydrophobic Non Amyloid beta Component (NAC) domain spanning residues 61-95; which is essential for alpha-synuclein fibrillization; and 3) the C-terminal region spanning residues 96-140 which is highly acidic and proline-rich and has no distinct structural propensity. Alpha-synuclein has been shown to undergo several posttranslational modifications, including truncations, phosphorylation, ubiquitination, oxidation and/or transglutaminase covalent cross linking (Fujiwara et al., Nat. Cell. Biol. 2002, 4(2); 160-164; Hasegawa et al., J. Biol. Chem. 2002, 277(50), 49071-49076; Li et al., Proc. Natl. Acad. Sci. USA 2005, 102(6), 2162-2167; Oueslati et al, Prog. Brain Res. 2010, 183, 115-145; Schmid et al., J. Biol. Chem. 2009, 284(19), 13128-13142). Interestingly, the majority of these modifications involve residues within the C-terminal region.

Several phosphorylation sites have been detected in the carboxyl-terminal region on Tyr-125, -133, and -136, and on Ser-129 (Negro et al., FASEB J. 2002, 16(2), 210-212). Tyr-125 residues can be phosphorylated by two Src family protein tyrosine kinases, c-Src and Fyn (Ellis et al., J. Biol. Chem. 2001, 276(6), 3879-3884; Nakamura et al., Biochem. Biophys. Res. Commun. 2001, 280(4), 1085-1092). Phosphorylation by Src family kinases does not suppress or enhance the tendency of alpha-synuclein to polymerize. Alpha-synuclein has proved to be an outstanding substrate for protein tyrosine kinase p72^{syk} (Syk) in vitro; once it is extensively Tyr-phosphorylated by Syk or tyrosine kinases with similar specificity, it loses the ability to form oligomers, suggesting a putative anti-neurodegenerative role for these tyrosine kinases (Negro et al., FASEB J. 2002, 16(2), 210-212).

Alpha-synuclein can be Ser-phosphorylated by protein kinases CKI and CKII (Okochi et al., J. Biol. Chem. 2000, 275(1), 390-397). The residue Ser-129 is also phosphorylated by G-protein-coupled receptor protein kinases (Pronin et al., J. Biol. Chem. 2000, 275(34), 26515-26522). Extensive and selective phosphorylation of alpha-synuclein at Ser-129 is evident in synucleinopathy lesions, including Lewy bodies (Fujiwara et al., Nat. Cell. Biol. 2002, 4(2); 160-164). Other post-translational modifications in the carboxyl-terminal, including glycosylation on Ser-129 (McLean et al., Neurosci. Lett. 2002, 323(3), 219-223) and nitration on Tyr-125, -133, and -136 (Takahashi et al., Brain Res. 2002, 938 (1-2), 73-80), may affect aggregation of alpha-synuclein. Truncation of the carboxyl-terminal region by proteolysis has been reported to play a role in alpha-synuclein fibrillogenesis in various neurodegenerative diseases (Rochet et al., Biochemistry 2000, 39(35), 10619-10626). Full-length as well as partially truncated and insoluble aggregates of alpha-synuclein have been detected in highly purified Lewy bodies (Crowther et al., FEBS Lett. 1998, 436(3), 309-312).

Abnormal protein aggregation appears to be a common feature in aging brain and in several neurodegenerative diseases (Trojanowski et al., 1998, Cell Death Differ. 1998, 5(10), 832-837, Koo et al., Proc. Natl. Acad. Sci. 1999, 96(18), 9989-9990, Hu et al., Chin. Sci. Bull. 2001, 46, 1-3); although a clear role in the disease process remains to be defined. In in vitro models, alpha-synuclein (or some of its truncated forms) readily assembles into filaments resembling those isolated from the brain of patients with Lewy Body (LB) dementia and familiar PD (Crowther et al., FEBS Lett. 1998, 436(3), 309-312). Alpha-synuclein and its mutated forms (A53T and A30P) have a random coil conformation and do not form significant secondary structures in aqueous solution at low concentrations; however, at higher concentrations they are prone to self-aggregate, producing amyloid fibrils (Wood et al., J. Biol. Chem. 1999, 274(28), 19509-19512). Several differences in the aggregation behavior of the PD-linked mutants and the wild-type protein have been documented. Monomeric alpha-synuclein aggregates in vitro form stable fibrils via a metastable oligomeric (i.e., protofibril) state (Volles et al., Biochemistry 2002, 41(14), 4595-4602).

Parkinson's disease (PD) is the most common neurodegenerative motor disorder. PD is mainly an idiopathic disease, although in at least 5% of the PD patients the pathology is linked to mutations in one or several specific genes. Several point mutations have been described in the alpha-synuclein gene (A30P, E46K, H50Q, G51D, A53T) which cause familial PD with autosomal dominant inheritance. Furthermore, duplications and triplications of the alpha-synuclein gene have been described in patients that developed PD, underlining the role of alpha-synuclein in PD pathogenesis (Lesage et al., Hum. Mol. Genet., 2009, 18, R48-59). The pathogenesis of PD remains elusive. However, growing evidence suggests a role for the pathogenic folding of the alpha-synuclein protein that leads to the formation of amyloid-like fibrils. Indeed, the hallmarks of PD are the presence of intracellular alpha-synuclein aggregate structures called Lewy Bodies and neurites mainly in the nigral neurons, as well as the death of dopaminergic neurons in the substantia nigra and elsewhere. Alpha-synuclein is a natively unfolded presynaptic protein that can misfold and aggregate into larger oligomeric and fibrillar forms which are linked to the pathogenesis of PD. Recent studies have implicated small soluble oligomeric and protofibrillar forms of alpha-synuclein as the most neurotoxic species (Lashuel et al., J. Mol. Biol., 2002, 322, 1089-102). However, the precise role of alpha-synuclein in the neuronal cell toxicity remains to be clarified (review: Cookson, Annu. Rev. Biochem., 2005, 74, 29-52).

Besides Parkinson's disease, the accumulation of aggregated alpha-synuclein into Lewy bodies is a characteristic of all Lewy body diseases, including Parkinson's disease with dementia (PDD), and dementia with Lewy bodies (DLB) (Capouch et al., Neurol. Ther. 2018, 7, 249-263). In DLB, Lewy Bodies are diffusely distributed throughout the cortices of the brain and in addition to Lewy Bodies and neurites, more threads and dot-like structures (Lewy dots) were found to be immunopositive for alpha-synuclein phosphorylated at Ser-129 (Outeiro et al., Mol. Neurodegener. 2019, 14, 5). Alpha-synuclein aggregates are also found in multiple system atrophy (MSA). MSA is a rare and sporadic neurodegenerative disorder that manifests with rapidly progressive autonomic and motor dysfunction, as well as variable cognitive decline. Such disorders include Shy-Drager syndrome, striatonigral degeneration and olivopontocerebellar atrophy. The disease can be clinically sub-classified in parkinsonian (MSA-P) or cerebellar (MSA-C) variant, depending on the predominant motor phenotype (Fanciulli et al., N. Engl. J. Med. 2015; 372, 249-63). It is characterized by the aggregation of alpha-synuclein in the cytoplasm of oligodendrocytes, forming glial cytoplasmic inclusions (GCIs). GCIs, consisting primarily of fibrillary forms of alpha-synuclein, are the neuropathological hallmark of MSA and are found throughout the neocortex, hippocampus, brainstem, spinal cord and dorsal root ganglia (Galvin et al., Arch Neurol. 2001, 58, 186-90). GCIs are considered a central player in the pathogenesis of MSA. A correlation between the GCI load and the degree of neuronal loss has been reported in both the striatonigral and the olivopontocerebellar regions (Stefanova et al., Neuropathol. Appl. Neurobiol. 2016, 42, 20-32). Furthermore, a causative link between GCIs and the induction of neuronal loss has been shown in transgenic mice overexpressing human alpha-synuclein in oligodendrocytes under various oligodendroglia-specific promoters. A key event in the pathophysiological cascade is considered to be the permissive templating (‘prion-like’ propagation) of misfolded alpha-synuclein.

The diagnosis of Parkinson's disease is largely clinical and depends on the presence of a specific set of symptoms and signs (the initial core feature being bradykinesia, rigidity, rest tremor and postural instability), the absence of atypical features, a slowly progressive course, and the response to a symptomatic drug therapy, mainly limited to a dopamine replacement therapy. The accurate diagnosis requires sophisticated clinical skills and is open to a degree of subjectivity and error, as several other degenerative and non-degenerative diseases can mimic PD symptoms (multiple system atrophy (MSA), progressive supranuclear palsy (PSP), Alzheimer's disease (AD), essential tremor, dystonic tremor), (Guideline No. 113: Diagnosis and pharmacological management of Parkinson's disease, January 2010. SIGN). The final confirmation of the pathology can only be made by post-mortem neuropathological analysis.

Computed tomography (CT) and conventional magnetic resonance imaging (MRI) brain scans of people with Parkinson's disease (PD) usually appear normal. These techniques are nevertheless useful to rule out other diseases that can be secondary causes of parkinsonism, such as basal ganglia tumors, vascular pathology and hydrocephalus. A specific technique of MRI, diffusion MRI, has been reported to be useful at discriminating between typical and atypical parkinsonism, although its exact diagnostic value is still under investigation. Dopaminergic function in the basal ganglia can be measured with different PET and SPECT radiotracers. Examples are ioflupane (¹²³I) (trade name DaTSCAN) and iometopane (Dopascan) for SPECT or fluorodeoxyglucose (¹⁸F) (¹⁸F-FDG) and dihydrotetrabenazine (¹¹C) (¹¹C-DTBZ) for PET. A pattern of reduced dopaminergic activity in the basal ganglia can aid in diagnosing PD, particularly in the symptomatic stage (Brooks, J. Nucl. Med., 2010, 51, 596-609; Redmond, Neuroscientist, 2002, 8, 457-88; Wood, Nat. Rev. Neurol., 2014, 10, 305).

Strategies are being developed to apply recent advances in understanding the potential causes of Parkinson's disease to the development of biochemical biomarkers (Schapira Curr. Opin. Neurol. 2013; 26(4):395-400). Such biomarkers that have been investigated in different body fluids (cerebrospinal fluid (CSF), plasma, saliva) include alpha-synuclein levels but also DJ-1, Tau and Abeta, as well as neurofilaments proteins, interleukins, osteopontin and hypocrontin (Schapira Curr. Opin. Neurol. 2013; 26(4):395-400), but so far none of these biomarkers alone or in combination can be used as a determinant diagnostic test. To our knowledge, no approved alpha-synuclein diagnostic agent is currently on the market or available for clinical trials despite a crucial need for Parkinson's disease research and drug development (Eberling et al., J Parkinsons Dis. 2013; 3(4):565-7).

The ability to image alpha-synuclein deposition in the brain would be a huge achievement for alpha-synucleopathies research, including Parkinson's disease research, diagnosis, and drug development. The accumulation of aggregated alpha-synuclein in the brain is considered a key pathological hallmark of Parkinson's disease (PD) and can start many years before the appearance of the symptoms. Therefore, alpha-synuclein is a priority target for drug development given not only its likely contribution to neurodegeneration but also because it can offer the possibility to treat the disease while still in the asymptomatic or prodromal stages. In vivo imaging of alpha-synuclein pathology could be useful as a biomarker to (i) detect the presence of the disease potentially in early stages, (ii) to evaluate disease progression and (iii) to be used as a pharmacodynamics tool for drug development. The development of an alpha-synuclein PET imaging agent is considered nowadays key for an accurate diagnosis of synucleinopathies as well as to support the clinical development of therapeutics targeting alpha-synuclein, starting from the optimal selection of the trial population (Eberling, Dave and Frasier, J. Parkinson's Disease, 3, 565-567 (2013)). Despite a huge effort to identify an alpha-synuclein PET ligand, so far only compounds that bind with reasonably high affinity to artificial alpha-synuclein fibrils were identified but none of them were confirmed in human clinical trials. They are not optimal for a number of reasons: low affinity or no binding was observed on pathological aggregates of alpha-synuclein present in the diseased brains, low or no selectivity for alpha-synuclein over other aggregated proteins was reported and inappropriate physicochemical properties for their use as brain-penetrant PET agents (Eberling et al., J Parkinsons Dis. 2013; 3(4):565-7; Neal et al., Mol. Imaging Biol. 2013, 15:585-595; Bagchi et al., PLoS One 2013, 8(2):e55031; Yu et al., Bioorganic and Medicinal chemistry 2012, 20:4625-4634; Zhang et al., Appl Sci (Basel) 2014, 4(1):66-78; Chu et al., J. Med. Chem., 2015, 58 (15):6002-17).

WO 2011/128455 refers to specific compounds which are suitable for treating disorders associated with amyloid proteins or amyloid-like proteins. US 2012/0302755 relates to certain imaging agents for detecting neurological dysfunction. Further compounds for the diagnosis of neurodegenerative disorders on the olfactory epithelium are discussed in WO 2012/037928.

WO 2010/063701 refers to a certain in vivo imaging agent for use in a method to determine the presence of, or susceptibility to, Parkinson's disease, wherein the in vivo imaging agent comprises an alpha-synuclein binder labelled with an in vivo imaging moiety, and wherein the in vivo imaging agent binds to alpha-synuclein with a binding affinity.

US 2014/0142089 relates to a method for preventing or treating a degenerative brain disease, the method comprising administering to a subject in need thereof an effective amount of a pharmaceutical composition comprising a specific compound, a pharmaceutically acceptable salt, an isomer, a solvate, a hydrate, and a combination thereof.

WO 2009/155017 describes aryl or heteroaryl substituted azabenzoxazole derivatives, which are stated to be useful as tracers in positron emission tomography (PET) imaging to study amyloid deposits in the brain in vivo to allow diagnosis of Alzheimer's disease.

WO 2016/033445 refers to a specific compound for imaging huntingtin protein.

WO 2017/153601 and WO 2019/234243 refer to bicyclic compounds for diagnosing alpha-synuclein aggregates.

Therefore, there is a need for a new class of imaging compounds that bind with high affinity to alpha-synuclein.

### SUMMARY OF THE INVENTION

The present invention provides compounds that can be employed in diagnosing a disease, disorder or abnormality associated with alpha-synuclein aggregates, including, but not limited to, Lewy bodies and/or Lewy neurites (such as Parkinson's disease), prognosing such a disease, disorder or abnormality, and monitoring the progression of such a disease, disorder or abnormality. In particular, the compounds should be suitable for determining a predisposition to such a disease, disorder or abnormality, monitoring the progression of the disease, disorder or abnormality, or predicting the responsiveness of a patient who is suffering from such a disease, disorder or abnormality to the treatment with a certain medicament. Furthermore, the compounds should be suitable for diagnosing a disease, disorder or abnormality associated with alpha-synuclein aggregates and/or detecting and optionally quantifying alpha-synuclein aggregates.

Various embodiments of the invention are described herein.

Within a certain aspect, provided herein is a compound of formula (I):

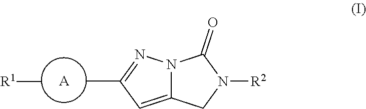

or a detectably labelled compound, stereoisomer, racemic mixture, pharmaceutically acceptable salt, hydrate, or solvate thereof, wherein

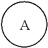

is a 6-membered heteroaryl, which is optionally substituted with at least one substituent independently selected from halo, or C₁-C₄alkyl;
R¹ is halo, haloC₁-C₄alkoxy, or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl.

In another aspect the invention is also directed to a compound having the following subformulae

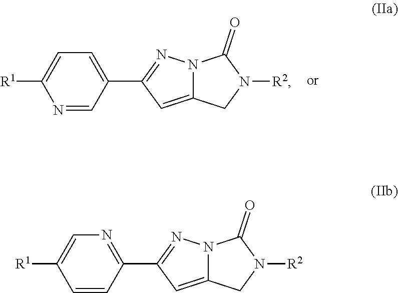

or a detectably labelled compound, stereoisomer, racemic mixture, pharmaceutically acceptable salt, hydrate, or solvate thereof.

In another aspect the invention is also directed to a compound having the following subformulae

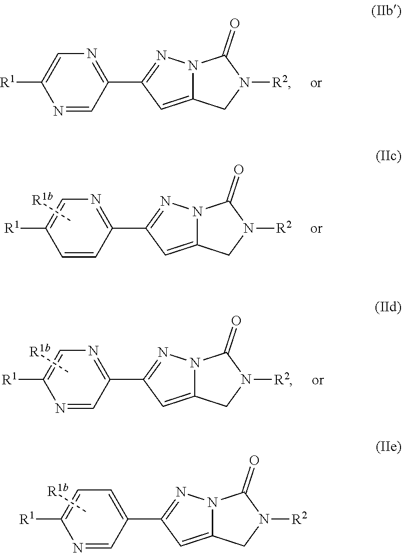

or a detectably labelled compound, stereoisomer, racemic mixture, pharmaceutically acceptable salt, hydrate, or solvate thereof.

In one aspect, the present invention provides a diagnostic composition comprising a compound of formula (I), and optionally at least one pharmaceutically acceptable excipient, carrier, diluent and/or adjuvant.

In one aspect, the present invention provides a compound of formula (I), or a diagnostic composition as defined herein, which can be use in the imaging of alpha-synuclein aggregates including, but not limited to, Lewy bodies and/or Lewy neurites. In another aspect the compound of formula (I), or the diagnostic composition can be for use in positron emission tomography imaging of alpha-synuclein aggregates including, but not limited to, Lewy bodies and/or Lewy neurites. In another aspect, the compound of formula (I) or the diagnostic composition, as defined herein, can be for use for in vitro imaging, ex vivo imaging, or in vivo imaging, preferably the use is for in vivo imaging, more preferably the use is for brain imaging. In yet another aspect, the compound of formula (I) or the diagnostic composition, as defined herein, can be use in diagnostics.

In a further aspect, the present invention refers to a method of diagnosing a disease, disorder or abnormality associated with alpha-synuclein aggregates including, but not limited to, Lewy bodies and/or Lewy neurites, in a subject, the method comprising the steps:
(a) Administering a compound of formula (I), or a diagnostic composition which comprises a compound of formula (I), as defined herein, to the subject;
(b) Allowing the compound to bind to the alpha-synuclein aggregates, including, but not limited to, Lewy bodies and/or Lewy neurites; and
(c) Detecting the compound bound to the alpha-synuclein aggregates, including, but not limited to, Lewy bodies and/or Lewy neurites.

In another aspect, the present invention refers to a method of positron emission tomography (PET) imaging of alpha-synuclein aggregates, including but not limited to, Lewy bodies and/or Lewy neurites, in a tissue of a subject, the method comprising the steps:
(a) Administering a compound of formula (I), or a diagnostic composition which comprises a compound of formula (I), as defined herein to the subject;
(b) Allowing the compound to bind to the alpha-synuclein aggregates, including, but not limited to, Lewy bodies and/or Lewy neurites; and
(c) Detecting the compound bound to the alpha-synuclein aggregates, including, but not limited to, Lewy bodies and/or Lewy neurites by collecting a positron emission tomography (PET) image of the tissue of the subject.

In a further aspect, the present invention is directed to a method for the detection and optionally quantification of alpha-synuclein aggregates, including, but not limited to, Lewy bodies and/or Lewy neurites, in a tissue of a subject, the method comprising the steps:
(a) Bringing a sample or a specific body part or body area suspected to contain alpha-synuclein aggregates, including, but not limited to, Lewy bodies and/or Lewy neurites, into contact with a compound of formula (I), or a diagnostic composition which comprises a compound of formula (I), as defined herein;
(b) Allowing the compound to bind to the alpha-synuclein aggregates, including, but not limited to, Lewy bodies and/or Lewy neurites;
(c) Detecting the compound bound to the alpha-synuclein aggregates, including, but not limited to, Lewy bodies and/or Lewy neurites; and
(d) Optionally quantifying the amount of the compound bound to the alpha-synuclein aggregates, including, but not limited to, Lewy bodies and/or Lewy neurites.

The present invention is also directed to a method of collecting data for the diagnosis of a disease, disorder or abnormality associated with alpha-synuclein aggregates including, but not limited to, Lewy bodies and/or Lewy neurites, wherein the method comprises the steps:
(a) Bringing a sample or a specific body part or body area suspected to contain alpha-synuclein aggregates including, but not limited to, Lewy bodies and/or Lewy neurites into contact with a compound of the formula (I), or a diagnostic composition which comprises a compound of formula (I), as defined herein;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites; and
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area.

The present invention also refers to a method of collecting data for determining a predisposition to a disease, disorder or abnormality associated with alpha-synuclein aggregates including, but not limited to, Lewy bodies and/or Lewy neurites, the method comprising the steps:
(a) Bringing a sample or a specific body part or body area suspected to contain alpha-synuclein aggregates including, but not limited to, Lewy bodies and/or Lewy neurites into contact with a compound of the formula (I), or a diagnostic composition which comprises a compound of formula (I), as defined herein;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites; and
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area.

In a further aspect the present invention also relates to a method of collecting data for prognosing a disease, disorder or abnormality associated with alpha-synuclein aggregates including, but not limited to, Lewy bodies and/or Lewy neurites, wherein the method comprises the steps:
(a) Bringing a sample, a specific body part or body area suspected to contain alpha-synuclein aggregates including, but not limited to, Lewy bodies and/or Lewy neurites into contact with a compound of the formula (I), or a diagnostic composition which comprises a compound of formula (I), as defined herein;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites;
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; and
(e) Optionally repeating steps (a) to (c) and, if present, optional step (d) at least one time.

In another aspect the present invention is directed to a method of collecting data for monitoring the progression of a disease, disorder or abnormality associated with alpha-synuclein aggregates including, but not limited to, Lewy bodies and/or Lewy neurites in a patient, the method comprising the steps:
(a) Bringing a sample, a specific body part or body area suspected to contain alpha-synuclein aggregates including, but not limited to, Lewy bodies and/or Lewy neurites into contact with the compound of the formula (I), or a diagnostic composition which comprises a compound of formula (I), as defined herein;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites;
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; and
(e) Optionally repeating steps (a) to (c) and, if present, optional step (d) at least one time.

In a further aspect, the present invention relates to a method of collecting data for predicting responsiveness of a patient suffering from a disease, disorder or abnormality associated with alpha-synuclein aggregates including, but not limited to, Lewy bodies and/or Lewy neurites to a medicament, the method comprising the steps:
(a) Bringing a sample, a specific body part or body area suspected to contain alpha-synuclein aggregates including, but not limited to, Lewy bodies and/or Lewy neurites into contact with a compound of formula (I), or a diagnostic composition which comprises a compound of formula (I), as defined herein;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites;
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; and
(e) Optionally repeating steps (a) to (c) and, if present, optional step (d) at least one time.

In another aspect the invention is further directed to a compound of formula (III-F) or (III-F′):

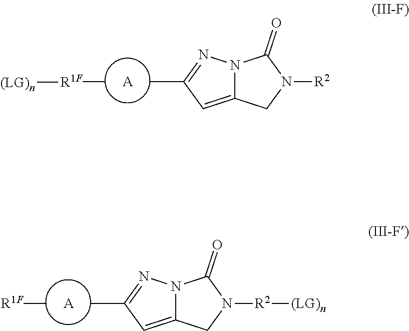

or a stereoisomer, racemic mixture, pharmaceutically acceptable salt, hydrate, or solvate thereof, wherein

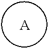

is a 6-membered heteroaryl which is optionally substituted with at least one substituent independently selected from halo, or C₁-C₄alkyl
R^{1F} is a 4- to 6-membered heterocyclyl, or C₁-C₄alkoxy and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
LG is a leaving group; and
n is at least 1.

In another aspect the invention is further directed to compound of formula (III-H)

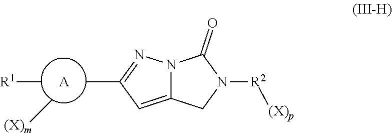

or a stereoisomer, racemic mixture, pharmaceutically acceptable salt, hydrate, or solvate thereof, wherein

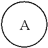

is a 6-membered heteroaryl, which is optionally substituted with at least one substituent independently selected from halo, or C₁-C₄alkyl;
R¹ is halo or a 4- to 6-membered heterocyclyl which is optionally substituted with at least one halo or haloC₁-C₄alkoxy;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
m is 0, 1, or 2;
p is 0, 1, or 2; and
X is bromo, chloro or iodo;
with the proviso that the compound of formula (III-H) comprises at least one X.

In another aspect, the invention is further directed to a method of preparing a compound of formula (I-F), by reacting a compound of formula (III-F) with a ¹⁸F-fluorinating agent, so that the Leaving Group (LG) is replaced by ¹⁸F.

In another aspect, the invention is further directed to a method of preparing a compound of formula (I-H), by reacting the compound of formula (III-H) with a ³H radiolabelling agent, so that X is replaced by ³H.

In another aspect, the invention is further directed to the use of the compound according to compound of formula (I) as an in vitro analytical reference or an in vitro screening tool.

In another aspect, the invention is further directed to a test kit for detection and/or diagnosis of a disease, disorder or abnormality associated with alpha-synuclein aggregates, wherein the test kit comprises at least one compound of formula (I) as defined herein.

The invention is further directed to a kit for preparing a radiopharmaceutical preparation, wherein the kit comprises a sealed vial containing at least one compound of formula (III-F) or (III-H).

### Definitions

For the purpose of interpreting this specification, the following definitions will apply unless specified otherwise, and when appropriate, terms used in the singular will also include the plural and vice versa. It must also be noted that as used herein and in the appended claims, the singular forms “a”, “an” and “the” include plural referents unless the context clearly dictates otherwise. Thus, for example, reference to “the compound” includes reference to one or more compounds; and so forth.

The term “C₁-C₄alkyl” refers to a saturated straight or branched hydrocarbon chain consisting solely of carbon and hydrogen atoms, containing no unsaturation, having from one to four carbon atoms, and which is attached to the rest of the molecule by a single bond. Examples of suitable alkyl groups having 1 to 4 carbon atoms include, but are not limited to, methyl, ethyl, propyl, isopropyl, 1-methylethyl, n-butyl, t-butyl and isobutyl.

The term “C₁-C₄alkoxy” refers to a radical of the formula —ORa where Ra is a C₁-C₄alkyl radical as generally defined above. Examples of C₁-C₄alkoxy include, but are not limited to, methoxy, ethoxy, propoxy, isopropoxy, butoxy, and isobutoxy.

The term “halogenC₁-C₄alkyl” or “haloC₁-C₄alkyl” refer to a C₁-C₄alkyl radical as defined above, substituted with one or more halo radicals as defined below. Examples of “haloC₁-C₄alkyl” include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

The term “halogenC₁-C₄alkoxy” refers to a C₁-C₄alkoxy radical as defined above, substituted with one or more halo radicals as defined below. Examples of “haloC₁-C₄alkoxy” include, but are not limited to, trifluoromethoxy, difluoromethoxy, fluoromethoxy, 2,2,2-trifluoroethoxy, 3,3,3-trifluoropropoxy, 4,4,4-trifluorobutoxy, 2,2-difluorobutoxy, and 4-bromobutoxy.

The term “heterocyclyl” refers to a stable 4- to 6-membered non-aromatic monocyclic ring radical which comprises 1 or 2 heteroatoms which are, e.g., selected from N, O or S. The heterocyclyl group can be unsaturated or saturated. The heterocyclyl radical may be bonded via a carbon atom or a heteroatom. Examples include, but are not limited to, azetidinyl, oxetanyl, pyrrolidinyl, pyrrolidyl, tetrahydrofuryl, tetrahydrothienyl, piperidyl, piperazinyl, tetrahydropyranyl, or morpholinyl, preferably azetidinyl, pyrrolidinyl, or piperidyl.

The term “heteroaryl” refers to a 5- or 6-membered aromatic monocyclic ring, which comprises 1, 2, or 3 heteroatoms independently selected from N, O and S. The heteroaryl radical may be bonded via a carbon atom or heteroatom selected from N, O and S. Examples of heteroaryl include, but are not limited to, thiopyranyl, dioxanyl, pyranyl, pyrazinyl, pyridazinyl, pyrimidyl or pyridyl.

The term “Hal” or “halogen” or “Halo” refers to F, Cl, Br, and L. With respect to diagnostic and pharmaceutical applications, F (e.g., ¹⁹F and ¹⁸F) is particularly preferred.

The term “leaving group” (LG) as employed herein is any leaving group and means an atom or group of atoms that can be replaced by another atom or group of atoms. Examples are given e.g. in Synthesis (1982), p. 85-125, table 2, Carey and Sundberg, Organische Synthese, (1995), page 279-281, table 5.8; or Netscher, Recent Res. Dev. Org. Chem., 2003, 7, 71-83, schemes 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33). Preferably, the “leaving group” (LG) is selected from halogen, C₁-C₄alkylsulfonate and C₆-C₁₀arylsulfonate, wherein the C₆-C₁₀arylsulfonate can be optionally substituted with —CH₃ or —NO₂.

Unless specified otherwise, the term “compound of the invention” refers to a compound of formula (I), or of subformulae thereof (e.g. (IIa), (IIb), (I-F), (I-H*), (I-H)), or a detectably labelled compound, stereoisomer (including diastereomeric mixtures and individual diastereomer, enantiomeric mixture and single enantiomer, mixture of conformers and single conformer), racemic mixture, pharmaceutically acceptable salt, hydrate, or solvate thereof. It is understood that every reference to a compound of formula (I) also covers the subformulae thereof (e.g. (IIa), (IIb), (I-F), (I-H*), (I-H)). The compounds of the formulae (III-F) and (III-H) will be referred to as the precursors of the compounds of the present invention.

Compounds of the present invention and their precursors having one or more optically active carbons can exist as racemates and racemic mixtures, stereoisomers (including diastereomeric mixtures and individual diastereomers, enantiomeric mixtures and single enantiomers, mixtures of conformers and single conformers), tautomers, atropoisomers, and rotamers. All isomeric forms are included in the present invention.

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds of the present invention and their precursors can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, PA, 1990, p. 1445, the disclosure of which is hereby incorporated by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Solvates” can be formed from the compound of the present invention and any suitable pharmaceutically acceptable solvent. Examples include C₁-4 alcohols (such as methanol or ethanol).

The patients or subjects in the present invention are typically animals, particularly mammals, more particularly humans.

Alpha-synuclein aggregates are multimeric beta-sheet rich assemblies of alpha-synuclein monomers that can form either soluble oligomers or soluble/insoluble protofibrils or mature fibrils which coalesce into intracellular deposits detected as a range of Lewy pathologies in Parkinson's disease and other synucleinopathies. Alpha-synuclein aggregates that are composing Lewy pathologies can be detected as having the following morphologies: Lewy bodies, Lewy neurites, premature Lewy bodies or pale bodies, perikaryal deposits with diffuse, granular, punctate or pleomorphic patterns. Moreover, alpha-synuclein aggregates are the major component of intracellular fibrillary inclusions detected in oligodendrocytes (also referred to as glial cytoplasmic inclusions) and in neuronal somata, axons and nuclei (referred to as neuronal cytoplasmic inclusions) that are the histological hallmarks of multiple system atrophy. Alpha-synuclein aggregates in Lewy pathologies often display substantial increase in post-translational modifications such as phosphorylation, ubiquitination, nitration, and truncation.

Lewy bodies are abnormal aggregates of protein that develop inside nerve cells in Parkinson's disease (PD), Lewy body dementia and other synucleinopathies. Lewy bodies appear as spherical masses that displace other cell components. Morphologically, Lewy bodies can be classified as being brainstem or cortical type. Classic brainstem Lewy bodies are eosinophilic cytoplasmic inclusions consisting of a dense core surrounded by a halo of 5-10-nm-wide radiating fibrils, the primary structural component of which is alpha-synuclein; cortical Lewy bodies differ by lacking a halo. The presence of Lewy bodies is a hallmark of Parkinson's disease.

Lewy neurites are abnormal neuronal processes in diseased neurons, containing granular material, abnormal alpha-synuclein (a-syn) filaments similar to those found in Lewy bodies, dot-like, varicose structures and axonal spheroids. Like Lewy bodies, Lewy neurites are a feature of α-synucleinopathies such as dementia with Lewy bodies, Parkinson's disease, and multiple system atrophy.

The terms “disease”, “disorder” or “abnormality” are used interchangeably herein.

The compounds of formula (I) can bind to alpha-synuclein aggregates. The type of bonding with the compounds of formula (I) has not been elucidated and any type of bonding is covered by the present invention. The wording “compound bound to the alpha-synuclein aggregates” and the like are used interchangeably herein and are not considered to be limited to any specific type of bonding.

The preferred definitions given in the “Definition”-section apply to all of the embodiments described below unless stated otherwise. Various embodiments of the invention are described herein, it will be recognized that features specified in each embodiment may be combined with other specified features to provide further embodiments of the present invention.

### BRIEF DESCRIPTION OF THE FIGURES

FIG. 1: Target engagement of [³H]-Example-1 on tissue from different alpha-synucleinopathies. Accumulation of silver grains on Lewy bodies and Lewy neurites, as shown in bottom panels. Immunofluorescence staining with a-syn-pS129 antibody was performed on the same sections, shown on top panels, to co-label alpha-synuclein aggregates. PD, Parkinson's Disease; PDD, Parkinson's Disease with Dementia; MSA, Multiple System Atrophy; DLB, Dementia with Lewy Bodies; LBV, Lewy Body Variant of Alzheimer's disease. Scale bar, 50 μm.

FIG. 2: Assessment of binding affinity of [³H]-Example-1 on human brain tissue from a familial PD case (G51D missense mutation) by autoradiography. A) Autoradiography images, B) Immunofluorescence staining with an a-syn-pS129 antibody, C) Specific binding of [³H]-Example-1, (counts per minute per mm²). Scale bar, 2 mm. ‘TB’, total binding; ‘NSB’, self-block, non-specific binding.

FIG. 3: Assessment of binding specificity of [³H]-Example-1 to diverse alpha-synucleinopathies and non-demented control cases by autoradiography. A) Autoradiography images; B) Immunofluorescence staining with an a-syn-pS129 antibody for the diseased donors. Scale bar, 5 mm. SNCA, alpha-synuclein [SNCA] gene G51D missense mutation; PDD, Parkinson's Disease with Dementia; LBV, Lewy Body Variant of Alzheimer's disease; MSA, Multiple System Atrophy; NDC, Non-Demented Control. ‘TB’, total binding; ‘NSB’, non-specific binding.

FIG. 4: Saturation binding with [³H]-Example 1 on PD brain-derived alpha-synuclein aggregates by micro-radiobinding. The plot displays specific binding, (counts per minute per mm²).

FIG. 5: Assessment of Kᵢ value of the compound of Example 1 for the displacement of reference Abeta compound ([³H]-Abeta-Ref) with non-radiolabelled compound of Example 1 on AD brain-derived homogenates. Percent competition values of [³H]-Abeta-Ref binding are plotted against increasing concentrations of non-radiolabelled compound of Example 1. Mean values of two independent experiments (with two technical replicates each) are shown.

FIG. 6: Assessment of target engagement of [³H]-Example-1 on AD tissue containing pathological Tau aggregates. No accumulation of silver grains on Tau tangles with [³H]-Example-1, as compared to a reference Tau ligand ([³H]-Tau-Ref).

### DETAILED DESCRIPTION OF THE INVENTION

The compounds of the present invention and their precursors are described in the following. It is to be understood that all possible combinations of the following definitions are also envisaged.

The invention relates to a compound of formula (I)

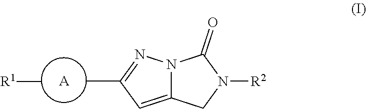

or a detectably labelled compound, stereoisomer, racemic mixture, pharmaceutically acceptable salt, hydrate, or solvate thereof, wherein

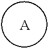

is a 6-membered heteroaryl, which is optionally substituted with at least one substituent independently selected from halo, or C₁-C₄alkyl;
R¹ is halo, haloC₁-C₄alkoxy, or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl.

In an embodiment the present invention relates to a compound of formula (I):

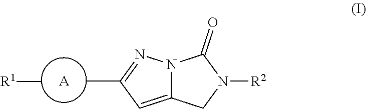

or a detectably labelled compound, stereoisomer, racemic mixture, pharmaceutically acceptable salt, hydrate, or solvate thereof, wherein

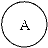

is a 6-membered heteroaryl;
R¹ is halo or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl.

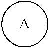

is a 6-membered heteroaryl, which is optionally substituted with at least one substituent independently selected from halo, or C₁-C₄alkyl. In one embodiment,

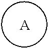

is a 6-membered heteroaryl.

In another embodiment, the invention provides a compound of formula (I) having a formula (IIa) or (IIb):

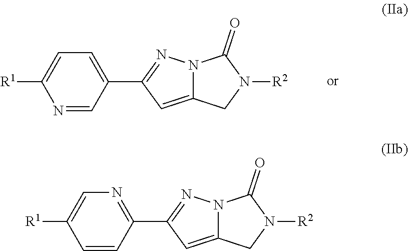

or a detectably labelled compound, stereoisomer, racemic mixture, pharmaceutically acceptable salt, hydrate, or solvate thereof.

In another embodiment, the invention provides a compound of formula (I) having a formula (IIb′) or (IIc) or (IId) or (IIe).

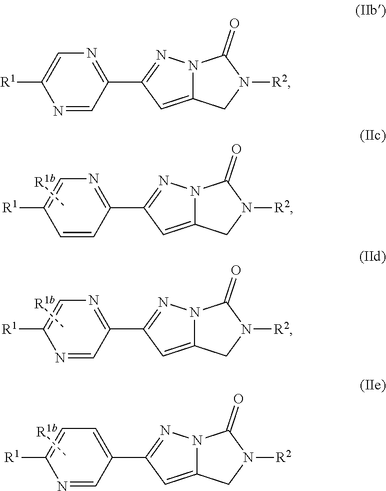

or a detectably labelled compound, stereoisomer, racemic mixture, pharmaceutically acceptable salt, hydrate, or solvate thereof, wherein R^{1b} is halo or C₁-C₄alkyl, preferably halo or CH₃. In one embodiment R^{1b} is halo, preferably F. Preferably F is ¹⁹F or ¹⁸F, even more preferably ¹⁸F. On another embodiment R^{1b} is CH₃.

In one embodiment, R¹ is halo or a 4- to 6-membered heterocyclyl which is optionally substituted with at least one halo. In one embodiment, R¹ is halo. In another embodiment, R¹ is a 4- to 6-membered heterocyclyl which is optionally substituted with at least one halo. In another embodiment R¹ is haloC₁-C₄alkoxy. In a preferred embodiment, R¹ is a 4- to 6-membered heterocyclyl which is substituted with at least one halo. Preferably, the heterocyclyl is substituted with at least one halo, more preferably with one or two halo, even more preferably with one halo. In one embodiment halo is F, and more preferably F is ¹⁹F or ¹⁸F, even more preferably ¹⁸F.

In one embodiment halo in R¹ and R^{1b} are F. Preferably F is ¹⁹F or ¹⁸F, more preferably ¹⁸F.

In one embodiment, R¹ is a 4- to 6-membered heterocyclyl selected from the following:

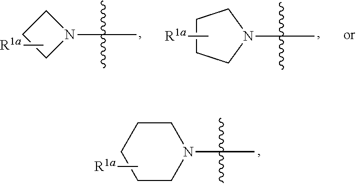

wherein R^{1a} is H or halo, preferably halo.

In a preferred embodiment, R¹ is a 4- to 5-membered heterocyclyl selected from the following:

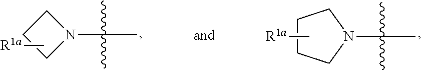

wherein R^{1a} is H or halo, preferably halo.

In a preferred embodiment, halo in R¹ and R^{1a} are F. Preferably, F is ¹⁹F or ¹⁸F, more preferably ¹⁸F.

In yet another embodiment R¹ is a 5-membered heterocyclyl which is:

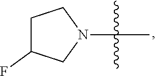 [SMILES: *N1CCC(F)C1]

preferably F is ¹⁹F or ¹⁸F, more preferably ¹⁸F.

In yet another embodiment R¹ is —O—(CH₂)ₘ-halo, wherein m is an integer from 1 to 4, preferably 1 or 2, more preferably 2.

In one embodiment, R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl, preferably haloC₁-C₄alkyl, or C₁-C₄alkyl.

In one preferred embodiment, R² is a 5-membered or 6-membered heteroaryl selected from the following:

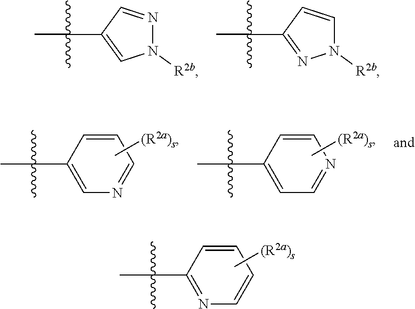

wherein
R^{2a} is independently selected from haloC₁-C₄alkyl, haloC₁-C₄alkoxy, C₁-C₄alkoxy, and C₁-C₄alkyl;
R^{2b} is selected from H, haloC₁-C₄alkyl, haloC₁-C₄alkoxy, C₁-C₄alkoxy, and C₁-C₄alkyl; and
s is 0, 1 or 2 (preferably 0 or 1).

In another embodiment, R² is a 5-membered heteroaryl selected from the following

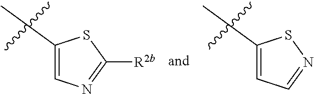

wherein
R^{2b} is selected from H, haloC₁-C₄alkyl, haloC₁-C₄alkoxy, C₁-C₄alkoxy, and C₁-C₄alkyl.

Preferably, R² is a 5-membered or 6-membered heteroaryl selected from the following:

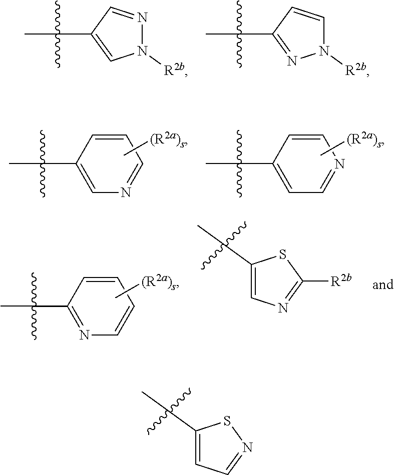

wherein
R^{2b} is selected from H, C₁-C₄alkyl, and haloC₁-C₄alkyl; and
s is 0.

In one preferred embodiment, R² is a 5-membered or 6-membered heteroaryl selected from the following:

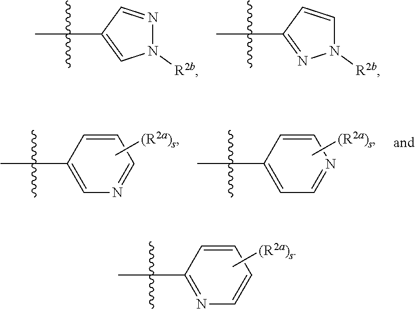

wherein
R^{2b} is selected from H, C₁-C₄alkyl, and haloC₁-C₄alkyl; and
s is 0.

In one embodiment, the present invention provides a compound of formula (I), wherein the compound is selected from

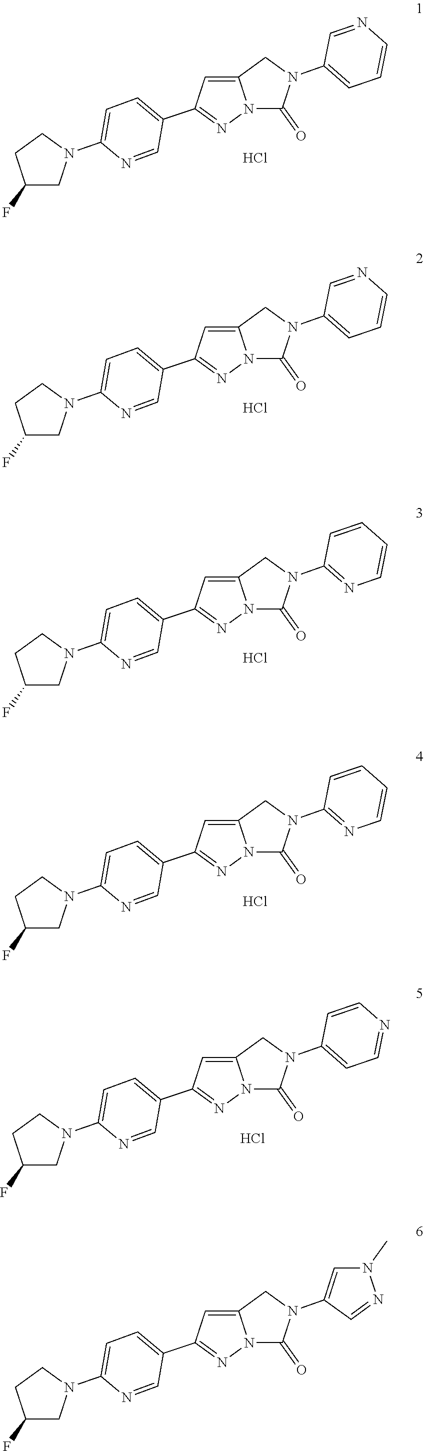

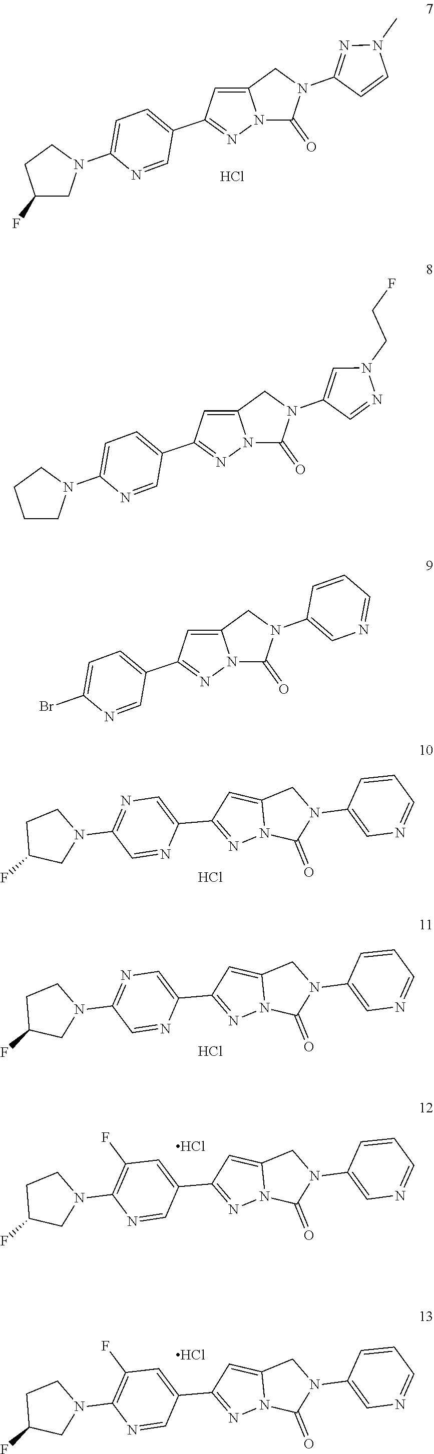

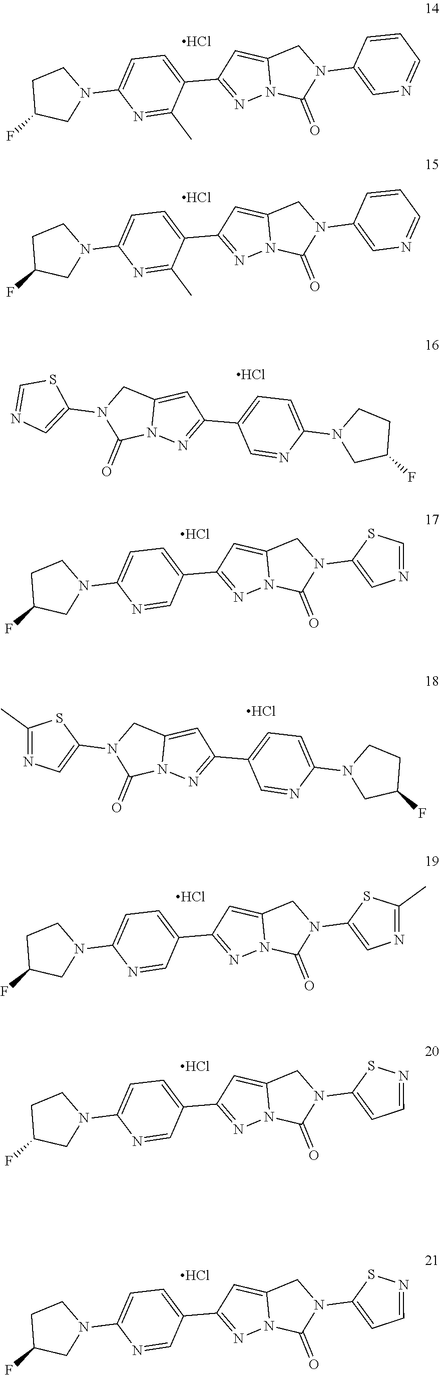

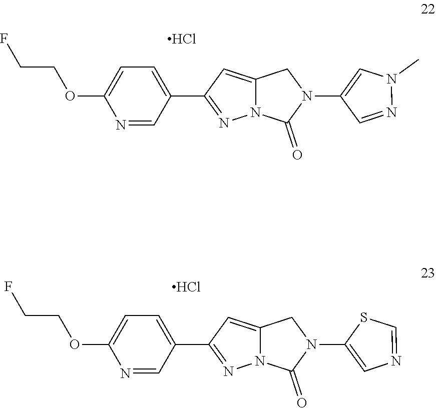 [SMILES: Cn1cc(N2Cc3cc(-c4ccc(OCCF)nc4)nn3C2=O)cn1 ; Cl ; O=C1N(c2cncs2)Cc2cc(-c3ccc(OCCF)nc3)nn21 ; Cl]

or a detectably labelled compound, stereoisomer, racemic mixture, pharmaceutically acceptable salt, hydrate, or solvate thereof.

In one embodiment, the present invention provides a compound of formula (I) wherein the compound of formula (I) is a detectably labelled compound. The detectable label can be a radioisotope. In one embodiment, the compound of formula (I) comprises at least one radioisotope. Preferably, the detectable label is a radioisotope selected from ¹⁸F, ²H and ³H. Most preferably, the radioisotope is selected from ¹⁸F and ³H.

In one embodiment the present invention provides a compound of formula (I), wherein the compound is a detectably labelled compound of formula (I-F) or (I-F′):

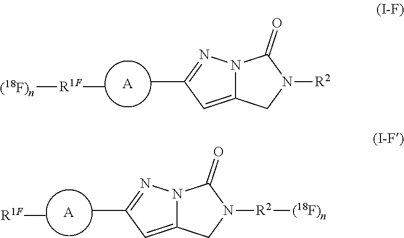

or a stereoisomer, racemic mixture, pharmaceutically acceptable salt, hydrate, or solvate thereof, wherein

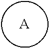

is a 6-membered heteroaryl which is optionally substituted with at least one substituent independently selected from halo, or C₁-C₄alkyl;
R^{1F} is a 4- to 6-membered heterocyclyl which is optionally substituted with at least one halo; or
R^{1F} is C₁-C₄alkoxy;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl; or and
n is at least 1, preferably 1.

In one embodiment the present invention provides a compound of formula (I), wherein the compound is a detectably labelled compound of formula (I-F) or (I-F′):

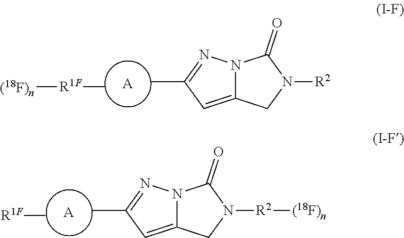

or a stereoisomer, racemic mixture, pharmaceutically acceptable salt, hydrate, or solvate thereof, wherein

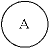

is a 6-membered heteroaryl;
R^{1F} is a 4- to 6-membered heterocyclyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl; preferably
R² is a 5-membered heteroaryl substituted with C₁-C₄alkyl and
n is at least 1, preferably 1.

In a preferred embodiment, —R^{1F}-(¹⁸F), is selected from the following:

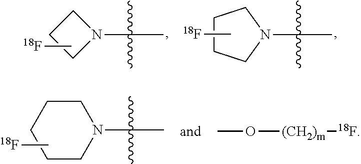

wherein m is at least 1, preferably 1 or 2, more preferably 2.

More preferably, —R^{1F}-(¹⁸F)ₙ is selected from the following:

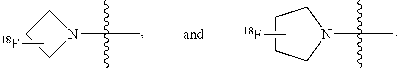

Even more preferably, —R^{1F}-(¹⁸F)ₙ is:

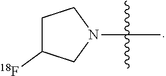 [SMILES: *N1CCC([18F])C1]

The detectably labelled compound of formula (I-F) or (I-F′) comprises at least one ¹⁸F. Preferably, the detectably labelled compound of formula (I-F) or (I-F′) comprises one or two ¹⁸F. Even more preferably, one ¹⁸F.

In one embodiment, the present invention provides a compound of formula (I), wherein the compound is a detectably labelled compound of formula (I-H*)

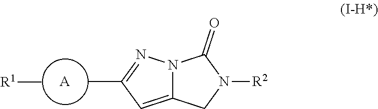

or a stereoisomer, racemic mixture, pharmaceutically acceptable salt, hydrate, or solvate thereof, wherein

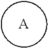

is a 6-membered heteroaryl, which is optionally substituted with at least one substituent independently selected from halo, or C₁-C₄alkyl;
R¹ is halo, haloC₁-C₄alkoxy or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl; with the proviso that the compound of formula (I-H*) comprises at least one ²H (deuterium “D”) or ³H (Tritium “T”), preferably T, preferably 1, 2, or 3 D or T.

In one embodiment, the present invention provides a compound of formula (I), wherein the compound is a detectably labelled compound of formula (I-H*)

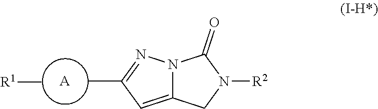

or a stereoisomer, racemic mixture, pharmaceutically acceptable salt, hydrate, or solvate thereof, wherein

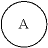

is a 6-membered heteroaryl;
R¹ is halo or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
with the proviso that the compound of formula (I-H*) comprises at least one ²H (deuterium “D”) or ³H (Tritium “T”), preferably T, preferably 1, 2, or 3 D or T.

In a preferred embodiment, the compound is a detectably labelled compound of formula (I-H)

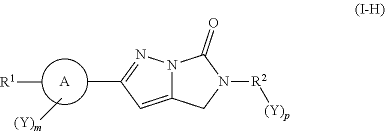

or a stereoisomer, racemic mixture, pharmaceutically acceptable salt, hydrate, or solvate thereof, wherein

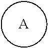

is a 6-membered heteroaryl, which is optionally substituted with at least one substituent independently selected from halo, or C₁-C₄alkyl;
R¹ is halo, haloC₁-C₄alkoxy or a 4- to 6-membered heterocyclyl which is optionally substituted with at least one halo;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;

### Y is T or CT₃;

m is 0, 1, 2 or 3;
p is 0, 1, 2 or 3;
with the proviso that the compound of formula (I-H) comprises at least one T or CT₃, wherein T is ³H (Tritium).

In a preferred embodiment, the compound is a detectably labelled compound of formula (I-H)

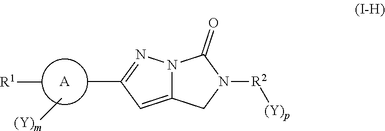

or a stereoisomer, racemic mixture, pharmaceutically acceptable salt, hydrate, or solvate thereof, wherein

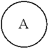

is a 6-membered heteroaryl;
R¹ is halo or a 4- to 6-membered heterocyclyl which is optionally substituted with at least one halo;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;

### Y is T or CT₃;

m is 0, 1, 2 or 3;
p is 0, 1, 2 or 3;
with the proviso that the compound of formula (I-H) comprises at least one T or CT₃, wherein T is ³H (Tritium).

It is understood that the tritium can present at any available position at which a hydrogen is present. For instance, in the group R² tritium can be present either directly bound to the 5-membered or 6-membered heteroaryl (such as in the form of T) or can be present in the haloC₁-C₄alkyl, haloC₁-C₄alkoxy, C₁-C₄alkoxy, and C₁-C₄alkyl (such as in the form of CT₃). In the 4- to 6-membered heterocyclyl of R¹ tritium can be, e.g., directly bound to the 4- to 6-membered heterocyclyl.

In one embodiment,

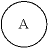

is a 6-membered heteroaryl and m is 1, 2 or 3, e.g., 1.

In one embodiment, R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl, and p is 1, 2 or 3, e.g., 1.

In a preferred embodiment, R² is a 5-membered or 6-membered heteroaryl selected from the following:

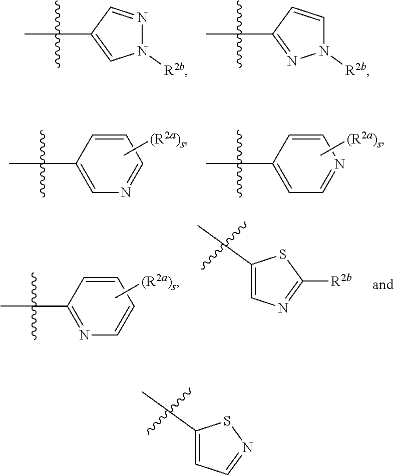

wherein
R^{2a} is independently selected from T, haloC₁-C₄alkyl, haloC₁-C₄alkoxy, C₁-C₄alkoxy, and C₁-C₄alkyl (e.g., CT₃);
R^{2b} is selected from H, T, haloC₁-C₄alkoxy, C₁-C₄alkoxy, haloalkyl and C₁-C₄alkyl;
s is 0, 1 or 2 (preferably 0 or 1); and
wherein haloC₁-C₄alkyl, haloC₁-C₄alkoxy, C₁-C₄alkyl, or C₁-C₄alkoxy optionally comprise one or more T.

In one embodiment, R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl, and p is 1, 2 or 3, e.g., 1.

In a preferred embodiment, R² is a 5-membered or 6-membered heteroaryl selected from the following:

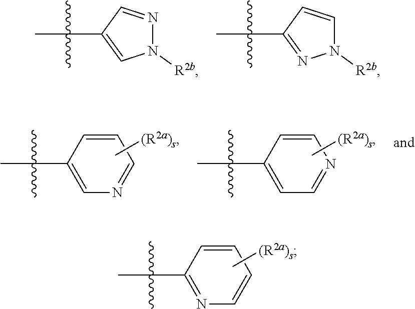

wherein
R^{2a} is independently selected from T, haloC₁-C₄alkyl, haloC₁-C₄alkoxy, C₁-C₄alkoxy, and C₁-C₄alkyl (e.g., CT₃);
R^{2b} is selected from H, T, haloC₁-C₄alkoxy, C₁-C₄alkoxy, haloalkyl and C₁-C₄alkyl (e.g., CT₃);
s is 0, 1 or 2 (preferably 0 or 1); and
wherein haloC₁-C₄alkyl, haloC₁-C₄alkoxy, C₁-C₄alkyl, or C₁-C₄alkoxy optionally comprise one or more T.

Preferably, R² is a 5-membered or 6-membered heteroaryl selected from the following:

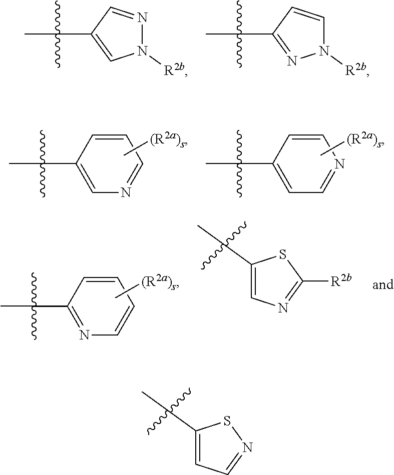

wherein

### R^{2a} is T;

R^{2b} is selected from H, T, haloC₁-C₄alkyl and C₁-C₄alkyl, wherein haloC₁-C₄alkyl and C₁-C₄alkyl optionally comprise one or more T (preferably R^{2b} is selected from T or CT₃); and
s is 0, 1 or 2 (preferably 1).

Preferably, R² is a 5-membered or 6-membered heteroaryl selected from the following:

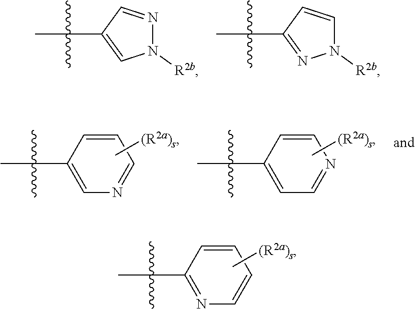

wherein

### R^{2a} is T or H;

R^{2b} is selected from H, haloC₁-C₄alkyl and C₁-C₄alkyl (e.g., CT₃), wherein haloC₁-C₄alkyl and C₁-C₄alkyl (preferably R^{2b} is selected from CT₃); and
s is 0, 1 or 2 (preferably 1).

Preferably, R^{2a} is -T, —OCH₃, —CH₃, -CT₃, or —H; and R^{2b} is selected from —H, -T or -CT₃.

In a preferred embodiment, the detectably labelled compound of formula (I-H*) or (I-H) comprises one, two or three T. Preferably, the detectably labelled compound of formula (I-H*) or (I-H) comprises one T. More preferably, the detectably labelled compound of formula (I-H*) or (I-H) comprises two T. Even more preferably, the detectably labelled compound of formula (I-H*) or (I-H) comprises three T such as -CT₃.

In another embodiment, the invention provides a detectably labelled compound of formula (I-H*) or (I-H) wherein ³H Tritium (“T”) can be replaced by ²H Deuterium (“D”). The deuterated compound can be prepared by reacting a compound of formula (III-H) with a ²H radiolabelling agent.

The compounds of the present invention and their precursors can be detectably labelled. The type of the label is not specifically limited and will depend on the detection method chosen. Examples of possible labels include isotopes such as radionuclides, positron emitters, and gamma emitters, preferably the detectable label is a radioisotope. With respect to the detectably labelled compounds of the present invention and their precursors which include a radioisotope, a positron emitter, or a gamma emitter, it is to be understood that the radioisotope, positron emitter, or gamma emitter is to be present in an amount which is not identical to the natural amount of the respective radioisotope, positron emitter, or gamma emitter. Furthermore, the employed amount should allow detection thereof by the chosen detection method. Examples of suitable isotopes such as radionuclides, positron emitters and gamma emitters include ²H, ³H, ¹⁸F, ¹C, ¹³N, and ¹⁵O, preferably ²H, ³H, ¹¹C, ¹³N, ¹⁵O, and ¹⁸F, more preferably ²H, ³H and ¹⁸F, even more preferably ³H and ¹⁸F. ¹⁸F-labelled compounds are particularly suitable for imaging applications such as PET. The corresponding compounds which include fluorine having a natural ¹⁹F isotope are also of particular interest as they can be used as analytical standards and references during manufacturing, quality control, release, and clinical use of their ¹⁸F-analogs.

Further, substitution with isotopes such as deuterium, i.e., ²H, may afford certain diagnostic and therapeutic advantages resulting from greater metabolic stability by reducing for example defluorination, increased in vivo half-life or reduced dosage requirements, while keeping or improving the original compound efficacy.

Isotopic variations of the compounds of the invention and their precursors can generally be prepared by conventional procedures such as by the illustrative methods or by the preparations described in the Examples and Preparative Examples hereafter using appropriate isotopic variations of suitable reagents, which are commercially available or prepared by known synthetic techniques.

Radionuclides, positron emitters and gamma emitters can be included into the compounds of the present invention and their precursors by methods which are usual in the field of organic synthesis. Typically, they will be introduced by using a correspondingly labelled starting material when the desired compound of the present invention and its precursor is prepared. Illustrative methods of introducing detectable labels are described, for instance, in US 2012/0302755.

The position at which the detectable label is to be attached to the compounds of the present invention and their precursors is not particularly limited. The radionuclides, positron emitters and gamma emitters, for example, can be attached at any position where the corresponding non-emitting atom can also be attached. For instance, ¹⁸F can be attached at any position which is suitable for attaching F. The same applies to the other radionuclides, positron emitters and gamma emitters. Due to the ease of synthesis, preferably R¹ is substituted with ¹⁸F. ³H can be attached at any available position at which H is present. If ²H is employed as a detectable label it can be attached at any available position at which H is present.

In another embodiment, the present invention relates further to a compound of formula (III-F) or (III-F′) that is a precursor of the compound of formula (I-F) and (I-F′), respectively

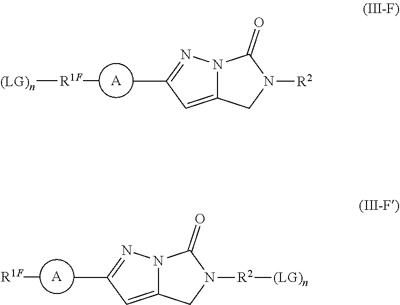

or a stereoisomer, racemic mixture, pharmaceutically acceptable salt, hydrate, or solvate thereof, wherein

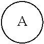

is a 6-membered heteroaryl which is optionally substituted with at least one substituent independently selected from halo, or C₁-C₄alkyl;
R^{1F} is a 4- to 6-membered heterocyclyl or C₁-C₄alkyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
LG is a leaving group; and
n is at least 1.

In another embodiment, the present invention relates further to a compound of formula (III-F) that is a precursor of the compound of formula (I-F)

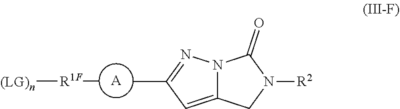

or a stereoisomer, racemic mixture, pharmaceutically acceptable salt, hydrate, or solvate thereof, wherein

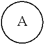

is a 6-membered heteroaryl;
R^{1F} is a 4- to 6-membered heterocyclyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
LG is a leaving group; and
n is at least 1.

In another preferred embodiment, (LG)ₙ-R^{1F} is selected from the following:

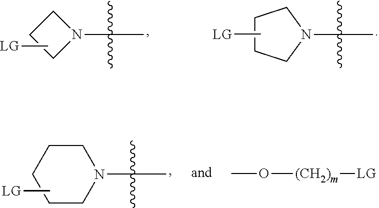

wherein m is at least 1, preferably 1 or 2, more preferably 2.

More preferably, (LG)ₙ-R^{1F} is selected from the following:

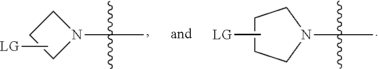

Even more preferably, (LG)ₙ-R^{1F} is:

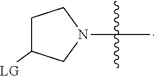

Preferably, the Leaving Group (LG) is halogen, C₁-C₄ alkylsulfonate, C₁-C₄alkyl ammonium, or C₆-C₁₀arylsulfonate, wherein the C₆-C₁₀arylsulfonate can be optionally substituted with —CH₃ or —NO₂. More preferably, the Leaving Group (LG) is bromo, chloro, iodo, C₆-C₄alkylsulfonate, or C₆-C₁₀arylsulfonate, wherein the C₆-C₁₀arylsulfonate can be optionally substituted with —CH₃ or —NO₂. Even more preferably, the Leaving Group (LG) is mesylate, tosylate or nosylate. Even more preferably, the Leaving Group (LG) is mesylate, or nosylate. More preferably the Leaving Group (LG) is mesylate.

In another embodiment, the present invention relates to a compound of formula (III-H), a precursor of the compound of formula (I-H):

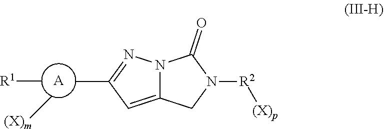

or a stereoisomer, racemic mixture, pharmaceutically acceptable salt, hydrate, or solvate thereof, wherein

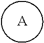

is a 6-membered heteroaryl which is optionally substituted with at least one substituent independently selected from halo, or C₁-C₄alkyl
R¹ is halo, haloC₁-C₄alkoxy, or a 4- to 6-membered heterocyclyl which is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
m is 0, 1, or 2;
p is 0, 1, or 2; and
X is bromo, chloro or iodo;
with the proviso that the compound of formula (III-H) comprises at least one X (e.g., 1, 2 or 3 X, preferably 1 or 2 X).

In another embodiment, the present invention relates to a compound of formula (III-H), a precursor of the compound of formula (I-H):

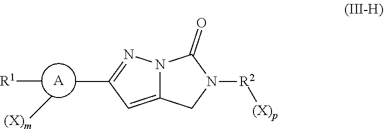

or a stereoisomer, racemic mixture, pharmaceutically acceptable salt, hydrate, or solvate thereof, wherein

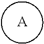

is a 6-membered heteroaryl;
R¹ is halo, or a 4- to 6-membered heterocyclyl which is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
m is 0, 1, or 2;
p is 0, 1, or 2; and
X is bromo, chloro or iodo;
with the proviso that the compound of formula (III-H) comprises at least one X (e.g., 1, 2 or 3 X, preferably 1 or 2 X).

In a preferred embodiment, (X)ₚ-R² is selected from the following:

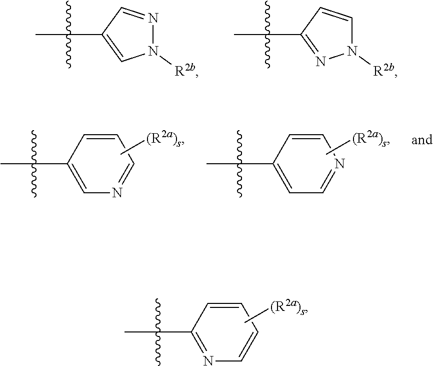

wherein
R^{2a} is independently selected from X, haloC₁-C₄alkyl, haloC₁-C₄alkoxy, C₁-C₄alkoxy, and C₁-C₄alkyl;
R^{2b} is selected from H, X, haloC₁-C₄alkoxy, C₁-C₄alkoxy, and C₁-C₄alkyl;
s is 0, 1 or 2 (preferably 0 or 1); and
wherein haloC₁-C₄alkyl, haloC₁-C₄alkoxy, C₁-C₄alkyl, or C₁-C₄alkoxy optionally comprises one or more X.

Preferably, (X)ₚ—R² is selected from the following:

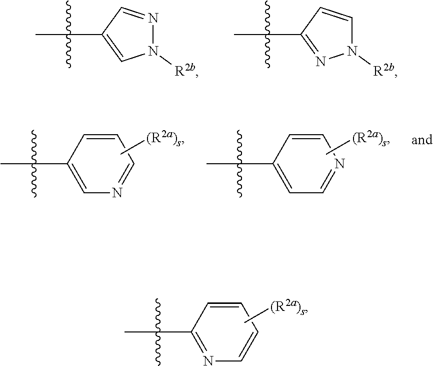

wherein

### R^{2a} is X;

R^{2b} is selected from H, X, haloC₁-C₄alkyl, and C₁-C₄alkyl, preferably X;
s is 0, 1 or 2 (preferably 1); and
wherein C₁-C₄alkyl, or haloC₁-C₄alkyl optionally comprises one or more X.

In a preferred embodiment, the detectably labelled compound of formula (III-H) comprises one, two or three X. In a preferred embodiment, the detectably labelled compound of formula (III-H) comprises one X. In another preferred embodiment, the detectably labelled compound of formula (III-H) comprises two X. In one embodiment, X is selected from bromo, chloro and iodo. In a preferred embodiment X is bromine.

### Methods of Synthesis of Detectably Labelled Compounds

The present invention relates further to a method for preparing a compound of formula (I), or of subformulae thereof (e.g. (IIa), (IIb), (I-F), (I-F′), (I-H*), (I-H)), and in particular a compound of formula (III-F), (III-F′), or (III-H) comprising a detectable label.

In one embodiment, the present invention relates to a method for preparing a compound of formula (I-F), by reacting a compound of formula (III-F) with a ¹⁸F-fluorinating agent.

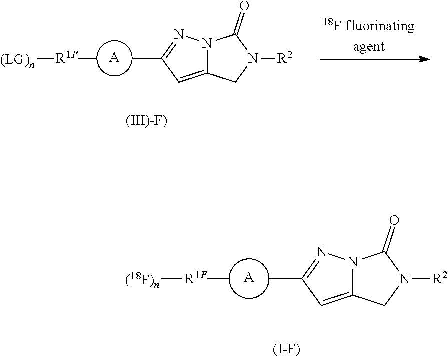

wherein

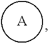

R^{1F}, R², n, and LG are as defined herein above.

In one embodiment, the present invention relates to a method for preparing a compound of formula (I-F′), by reacting a compound of formula (III-F′) with a ¹⁸F-fluorinating agent.

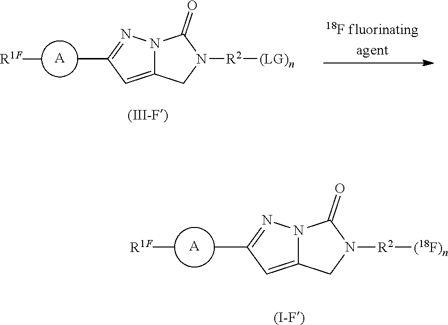

wherein

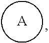

R^{1F}, R², n, and LG are as defined herein above. Suitable solvents for the ¹⁸F-fluorination comprise DMF, DMSO, acetonitrile, DMA, or mixtures thereof, preferably acetonitrile or DMSO. Suitable agents for the ¹⁸F-fluorination are selected from K¹⁸F, Rb¹⁸F, Cs¹⁸F, Na¹⁸F, tetra(C₁₋₆alkyl)ammonium salt of ¹⁸F, Kryptofix[222]¹⁸F and tetrabutylammonium [¹⁸F]fluoride.

In one embodiment, the present invention relates to a method of preparing a compound of formula (I-H), by reacting a compound of formula (III-H) with a ³H radiolabeling agent.

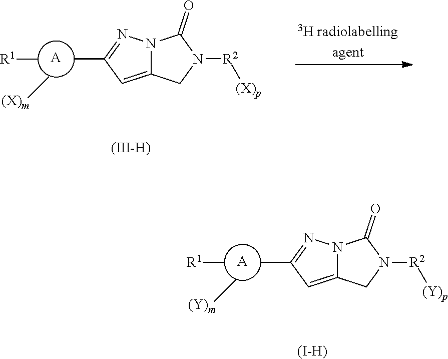

wherein

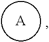

R¹, R², X, Y, m, and p are as defined herein above.

The ³H radiolabeling agent can be tritium gas. The method can be conducted in the presence of a catalyst such as palladium on carbon (Pd/C), a solvent such as dimethylformamide (DMF) and a base such as N,N-diisopropylethylamine (DIEA).

Alternatively, in another embodiment, the present invention relates to a method for preparing a compound of formula (I-H), by radiolabeling a compound of formula (III-H) with a CT₃ radiolabeling agent, wherein T is ³H. The CT₃ radiolabeling agent can be ICT₃ (derivative of iodomethane with ³H). The method can be conducted in the presence of a solvent such as dimethylformamide (DMF) and a base such as cesium carbonate or sodium hydride.

### Radiopharmaceutical Preparations

The compounds of the present invention can also be employed in kits for the preparation of radiopharmaceutical preparations. Due to the radioactive decay, the radiopharmaceuticals are usually prepared immediately before use. The kit typically comprises a precursor of the compound of the present invention, and an agent which reacts with the precursor to introduce a radioactive label into the compound of the present invention. The precursor of the compound of the present invention, can, for example, be a compound having the formula (III-F), or (III-H). The agent can be an agent which introduces a radioactive label such as ¹⁸F, or ³H.

In one embodiment, the kit of part is a test kit for the detection and/or diagnosis of a disease, disorder or abnormality associated with alpha-synuclein aggregates, wherein the test kit comprises at least one precursor of the compound of the present invention (e.g. a compound having the formula (III-F) or (III-H)).

In another embodiment, the kit of part is a kit for preparing a radiopharmaceutical preparation, wherein the kit comprises a sealed vial containing at least one precursor of the compound of the present invention (e.g. a compound having the formula (III-F) or (III-H)).

### Diagnostic Compositions

The compounds of the present invention are particularly suitable for imaging of alpha-synuclein aggregates including, but not limited to, Lewy bodies and/or Lewy neurites. With respect to alpha-synuclein protein, the compounds are particularly suitable for binding to various types of alpha-synuclein aggregates including, but not limited to, Lewy bodies and/or Lewy neurites. The imaging can be conducted in mammals, preferably in humans. The imaging is preferably in vitro imaging, ex vivo imaging, or in vivo imaging. More preferably the imaging is in vivo imaging: Even more preferably, the imaging is preferably brain imaging. The imaging can also be eye/retinal imaging. The compounds of the present invention are particularly suitable for use in diagnostics.

The diagnostics can be conducted for mammals, preferably for humans. The tissue of interest on which the diagnostic is conducted can be brain, tissue of the central nervous system, tissue of the eye (such as retinal tissue), tissue of peripheral organs such as the gut or other tissues, or body fluids such as cerebrospinal fluid (CSF) or blood. The tissue is preferably brain tissue.

In one embodiment, the present invention provides a diagnostic composition comprising a compound of the invention, and optionally at least one pharmaceutically acceptable excipient, carrier, diluent and/or adjuvant.

Due to their design and to the binding characteristics, the compounds of the present invention are suitable for use in the diagnosis of diseases, disorders and abnormalities associated with alpha-synuclein aggregates including, but not limited to, Lewy bodies and/or Lewy neurites. In another embodiment, the diagnostic composition which comprises a compound of the present invention is also suitable for use in the diagnosis of diseases, disorders and abnormalities associated with alpha-synuclein aggregates including, but not limited to, Lewy bodies and/or Lewy neurites.

In yet another embodiment, the compound of the present invention, or the diagnostic composition comprising a compound of the invention, is suitable for use in imaging, such as in vitro imaging, ex vivo imaging, or in vivo imaging, preferably the use is for in vivo imaging, more preferably the use is for brain imaging. In particular, the use is in humans.

In another embodiment, the compounds of the present invention or the diagnostic composition are particularly suitable for use in positron emission tomography imaging of alpha-synuclein aggregates including, but not limited to, Lewy bodies and/or Lewy neurites.

Diseases involving alpha-synuclein aggregates are generally listed as synucleinopathies (or α-synucleinopathies). The compounds of the present invention are suitable for use in the diagnosis of diseases, disorders or abnormalities including, but not limited to, Parkinson's disease (sporadic, familial with alpha-synuclein mutations, familial with mutations other than alpha-synuclein, pure autonomic failure and Lewy body dysphagia), SNCA duplication carrier, dementia with Lewy bodies (“pure” Lewy body dementia), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease and normal aging in Down syndrome). Synucleinopathies with neuronal and glial aggregates of alpha synuclein include multiple system atrophy (MSA) (Shy-Drager syndrome, striatonigral degeneration and olivopontocerebellar atrophy). Other diseases that may have alpha-synuclein-immunoreactive lesions include traumatic brain injury, chronic traumatic encephalopathy, tauopathies (Pick's disease, frontotemporal dementia, progressive supranuclear palsy, corticobasal degeneration and Niemann-Pick type C₁ disease), motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, ataxia telangiectatica, Meige's syndrome, subacute sclerosing panencephalitis, Gaucher disease as well as other lysosomal storage disorders (including Kufor-Rakeb syndrome and Sanfilippo syndrome) and rapid eye movement (REM) sleep behavior disorder (Jellinger, Mov Disord 2003, 18 Suppl. 6, S2-12; Galvin et al. JAMA Neurology 2001, 58 (2), 186-190; Kovari et al., Acta Neuropathol. 2007, 114(3), 295-8; Saito et al., J Neuropathol Exp Neurol. 2004, 63(4), 323-328; McKee et al., Brain, 2013, 136 (Pt 1), 43-64; Puschmann et al., Parkinsonism Relat Disord 2012, 18S1, S24-S27; Usenovic et al., J Neurosci. 2012, 32(12), 4240-4246; Winder-Rhodes et al., Mov Disord. 2012, 27(2), 312-315; Ferman et al., J Int Neuropsychol Soc. 2002, 8(7), 907-914). Preferably, the compounds of the present invention are suitable for use in the diagnosis of Parkinson's disease, multiple system atrophy, dementia with Lewy bodies, Parkinson's disease dementia, SNCA duplication carrier, or Alzheimer's disease, more preferably Parkinson's disease (PD).

In the methods of diagnosing a disease, disorder or abnormality associated with alpha-synuclein aggregates including, but not limited to, Lewy bodies and/or Lewy neurites (e.g. Parkinson's disease), or a predisposition therefor in a subject, the method comprises the steps of:
(a) administering to the subject a diagnostically effective amount of a compound of the present invention, or a diagnostic composition which comprises a compound of the present invention;
(b) allowing the compound of the present invention to distribute into the tissue of interest (such as brain tissue, tissue of the central nervous system (CNS), tissue of the eye, tissue of peripheral organs or other tissues), or body fluid (such as cerebrospinal fluid (CSF) or blood); and
(c) imaging the tissue of interest or body fluid.

If the amount of the compound bound to the alpha-synuclein aggregates, including, but not limited to, Lewy bodies and/or Lewy neurites is increased compared to a normal control level the subject is suffering from or is at risk of developing a disease, disorder or abnormality associated with alpha-synuclein aggregates including, but not limited to, Lewy bodies and/or Lewy neurites.

The compounds of the present invention can be used for imaging of alpha-synuclein aggregates including, but not limited to, Lewy bodies and/or Lewy neurites in any sample or a specific body part or body area of a patient which is suspected to contain alpha-synuclein aggregates including, but not limited to, Lewy bodies and/or Lewy neurites. The compounds are able to pass the blood-brain barrier. Consequently, they are particularly suitable for imaging of alpha-synuclein aggregates including, but not limited to, Lewy bodies and/or Lewy neurites in the brain, tissue of the central nervous system (CNS), tissue of the eye (such as retinal tissue), tissue of peripheral organs such as the gut or other tissues, or body fluids such as cerebrospinal fluid (CSF) or blood.

In diagnostic applications, the compounds of the present invention are preferably administered in the form of a diagnostic composition comprising the compound of the invention. A “diagnostic composition” is defined in the present invention as a composition comprising one or more compounds of the present invention in a form suitable for administration to a patient, e.g., a mammal such as a human, and which is suitable for use in the diagnosis of the specific disease, disorder or abnormality at issue. Preferably a diagnostic composition further comprises a pharmaceutically acceptable excipient, carrier, diluent or adjuvant. Administration is preferably carried out as defined below. More preferably by injection of the composition as an aqueous solution. Such a composition may optionally contain further ingredients such as buffers; pharmaceutically acceptable solubilizers (e.g., cyclodextrins or surfactants such as Pluronic, Tween or phospholipids); and pharmaceutically acceptable stabilisers or antioxidants (such as ascorbic acid, gentisic acid or para-aminobenzoic acid). The dose of the compound of the present invention will vary depending on the exact compound to be administered, the weight of the patient, and other variables as would be apparent to a physician skilled in the art.

While it is possible for the compounds of the present invention to be administered alone, it is preferable to formulate them into a diagnostic composition in accordance with standard pharmaceutical practice. Thus, the invention also provides a diagnostic composition which comprises a diagnostically effective amount of a compound of the present invention in admixture with, optionally, at least one pharmaceutically acceptable excipient, carrier, diluent or adjuvant.

Pharmaceutically acceptable excipients are well known in the pharmaceutical art, and are described, for example, in Remington's Pharmaceutical Sciences, 15^{th} Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

Pharmaceutically useful excipients, carriers, adjuvants and diluents that may be used in the formulation of the diagnostic composition of the present invention may comprise, for example, solvents such as monohydric alcohols such as ethanol, isopropanol and polyhydric alcohols such as glycols and edible oils such as soybean oil, coconut oil, olive oil, safflower oil, cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone, low melting waxes, and ion exchange resins.

The routes for administration (delivery) of the compounds of the invention include, but are not limited to, one or more of: intravenous, gastrointestinal, intraspinal, intraperitoneal, intramuscular, oral (e. g. as a tablet, capsule, or as an ingestible solution), topical, mucosal (e. g. as a nasal spray or aerosol for inhalation), nasal, parenteral (e. g. by an injectable form), intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Preferably, the route of administration (delivery) of the compounds of the invention is intravenous.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include starch, a cellulose, milk sugar (lactose) or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Preferably, in diagnostic applications, the compounds of the present invention are administered parenterally. If the compounds of the present invention are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e. g. using a mixture of ethanol and the propellant as the solvent, which may additionally contain a lubricant, e. g. sorbitan trioleate. Capsules and cartridges (made, for example, from gelatin) for use in an inhaler or insufflator may be formulated to contain a powder mix of the compound and a suitable powder base such as lactose or starch.

Alternatively, the compounds of the present invention can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH was adjusted, sterile saline, or, preferably, as solutions in isotonic, pH was adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing diagnosis.

The diagnostic compositions of the invention can be produced in a manner known per se to the skilled person as described, for example, in Remington's Pharmaceutical Sciences, 15^{th} Ed., Mack Publishing Co., New Jersey (1975).

The compounds of the present invention are useful as an in vitro analytical reference or an in vitro screening tool. They are also useful in in vivo diagnostic methods.

The compounds according to the present invention can also be provided in the form of a mixture, a pharmaceutical composition, or a combination, comprising a compound according to the present invention and at least one compound selected from an imaging agent different from the compound according to the invention, a pharmaceutically acceptable excipient, carrier, diluent or adjuvant. The imaging agent different from the compound according to the invention is preferably present in a diagnostically effective amount. More preferably the imaging agent different from the compound according to the invention is an Abeta or Tau imaging agent.

### Methods

In one embodiment, the invention provides a method of diagnosing a disease, disorder or abnormality associated with alpha-synuclein aggregates including, but not limited to, Lewy bodies and/or Lewy neurites, in a subject, the method comprising the steps:
(a) Administering a compound of the invention, or a diagnostic composition which comprises a compound of the invention to the subject;
(b) Allowing said compound to bind to the alpha-synuclein aggregates, including, but not limited to, Lewy bodies and/or Lewy neurites; and
(c) Detecting the compound bound to the alpha-synuclein aggregates, including, but not limited to, Lewy bodies and/or Lewy neurites.

Optionally, said method may further comprise the step of:
(d) Generating an image representative of the location and/or amount of the compound bound to the alpha-synuclein aggregates including, but not limited to, Lewy bodies and/or Lewy neurites.

In another embodiment, the invention provides a method of positron emission tomography (PET) imaging of alpha-synuclein aggregates, including but not limited to, Lewy bodies and/or Lewy neurites, in a tissue of a subject, the method comprising the steps:
(a) Administering a compound of the invention, or a diagnostic composition which comprises a compound of the invention to the subject;
(b) Allowing the compound to bind to the alpha-synuclein aggregates, including, but not limited to, Lewy bodies and/or Lewy neurites; and
(c) Detecting the compound bound to the alpha-synuclein aggregates, including, but not limited to, Lewy bodies and/or Lewy neurites by collecting a positron emission tomography (PET) image of the tissue of the subject;

In another embodiment, the invention relates to a method for the detection and optionally quantification (e.g., an in vivo or in vitro method) of alpha-synuclein aggregates, including but not limited to, Lewy bodies and/or Lewy neurites, in a tissue of a subject, the method comprising the steps:
(a) Bringing a sample or a specific body part or body area suspected to contain alpha-synuclein aggregates, including but not limited to, Lewy bodies and/or Lewy neurites, into contact with a compound of the invention, or a diagnostic composition which comprises a compound of the invention;
(b) Allowing the compound to bind to the alpha-synuclein aggregates, including but not limited to, Lewy bodies and/or Lewy neurites;
(c) Detecting the compound bound to the alpha-synuclein aggregates, including but not limited to, Lewy bodies and/or Lewy neurites; and
(d) Optionally quantifying the amount of the compound bound to the alpha-synuclein aggregates, including but not limited to, Lewy bodies and/or Lewy neurites.

In an embodiment, the present invention refers to a method of collecting data for the diagnosis of a disease, disorder or abnormality associated with alpha-synuclein aggregates including, but not limited to, Lewy bodies and/or Lewy neurites, the method comprising the steps:
(a) Bringing a sample or a specific body part or body area suspected to contain alpha-synuclein aggregates including, but not limited to, Lewy bodies and/or Lewy neurites into contact with a compound according to the present invention, or a diagnostic composition which comprises a compound according to the present invention;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites; and
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area.

If the amount of the compound bound to the alpha-synuclein aggregates including, but not limited to, Lewy bodies and/or Lewy neurites is higher than a normal control value it can be assumed that the patient is suffering from a disease, disorder or abnormality associated with alpha-synuclein aggregates including, but not limited to, Lewy bodies and/or Lewy neurites.

Yet another embodiment of the present invention refers to a method of collecting data for determining a predisposition to a disease, disorder or abnormality associated with alpha-synuclein aggregates including, but not limited to, Lewy bodies and/or Lewy neurites, the method comprising the steps:
(a) Bringing a sample or a specific body part or body area suspected to contain alpha-synuclein aggregates including, but not limited to, Lewy bodies and/or Lewy neurites into contact with a compound according to the present invention, or a diagnostic composition which comprises a compound according to the present invention;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites; and
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area.

If the amount of the compound bound to the alpha-synuclein aggregates is higher than a normal control value of a healthy/reference subject this indicates that the patient is suffering from or is at risk of developing a disease, disorder or abnormality associated with alpha-synuclein aggregates. In particular, if the amount of the compound bound to the alpha-synuclein aggregates is higher than what expected in a person showing no clinical evidence of a disease, disorder or abnormality associated with alpha-synuclein aggregates including, but not limited to, Lewy bodies and/or Lewy neurites, it can be assumed that the patient has a disposition to a disease, disorder or abnormality associated with alpha-synuclein aggregates.

In a further aspect, the present invention relates to a method of collecting data for prognosing a disease, disorder or abnormality associated with alpha-synuclein aggregates including, but not limited to, Lewy bodies and/or Lewy neurites, wherein the method comprises the steps:
(a) Bringing a sample, a specific body part or body area suspected to contain alpha-synuclein aggregates including, but not limited to, Lewy bodies and/or Lewy neurites into contact with a compound according to the present invention, or a diagnostic composition which comprises a compound according to the present invention;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites;
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; and
(e) Optionally repeating steps (a) to (c) and, if present, optional step (d) at least one time.

The progression of a disease, disorder or abnormality and/or the prospect (e.g., the probability, duration, and/or extent) of recovery can be estimated by a medical practitioner based on the presence or absence of the compound bound to the alpha-synuclein aggregates, the amount of the compound bound to the alpha-synuclein aggregates or the like. If desired, steps (a) to (c) and, if present, optional step (d) can be repeated over time to monitor the progression of the disease, disorder or abnormality and to thus allow a more reliable estimate.

A further aspect is directed to a method of collecting data for monitoring the progression (or evolution) of a disease, disorder or abnormality associated with alpha-synuclein aggregates including, but not limited to, Lewy bodies and/or Lewy neurites in a patient, the method comprising the steps:
(a) Bringing a sample, a specific body part or body area suspected to contain alpha-synuclein aggregates including, but not limited to, Lewy bodies and/or Lewy neurites into contact with the compound according to the present invention, or a diagnostic composition which comprises a compound according to the present invention;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites;
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; and
(e) Optionally repeating steps (a) to (c) and, if present, optional step (d) at least one time.

In the method for monitoring the progression the amount of the compound bound to the alpha-synuclein aggregates can be optionally compared at various points of time during the treatment, for instance, before and after onset of the treatment or at various points of time after the onset of the treatment.

Typically, the patient is or has been undergoing treatment of the disease, disorder or abnormality associated with alpha-synuclein aggregates or is/has been undergoing treatment of the synucleinopathy. In particular, the treatment can involve administration of a medicament which is suitable for treating the disease, disorder or abnormality associated with alpha-synuclein aggregates.

In another embodiment, the invention relates to a method of collecting data for predicting responsiveness of a patient suffering from a disease, disorder or abnormality associated with alpha-synuclein aggregates including, but not limited to, Lewy bodies and/or Lewy neurites to a treatment with a medicament, the method comprising the steps of
(a) Bringing a sample, a specific body part or body area suspected to contain alpha-synuclein aggregates including, but not limited to, Lewy bodies and/or Lewy neurites into contact with a compound of the invention, or a diagnostic composition which comprises a compound of the invention;
(b) Allowing the compound to bind to the alpha-synuclein aggregates including, but not limited to, Lewy bodies and/or Lewy neurites;
(c) Detecting the compound bound to the alpha-synuclein aggregates including, but not limited to, Lewy bodies and/or Lewy neurites;
(d) Optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; and
(e) Optionally repeating steps (a) to (c) and, if present, optional step (d) at least one time.

In the method for predicting the responsiveness, the method can further comprises steps (i) to (vi) before step (a):
(i) bringing a sample or specific body part or body area suspected to contain alpha-synuclein aggregates including, but not limited to, Lewy bodies and/or Lewy neurites into contact with the compound of the present invention, which compound specifically binds to the alpha-synuclein aggregates including, but not limited to, Lewy bodies and/or Lewy neurites;
(ii) allowing the compound to bind to the alpha-synuclein aggregates including, but not limited to, Lewy bodies and/or Lewy neurites;
(iii) detecting the formation of the compound bound to the alpha-synuclein aggregates including, but not limited to, Lewy bodies and/or Lewy neurites;
(iv) optionally correlating the presence or absence of the compound bound to the alpha-synuclein aggregates including, but not limited to, Lewy bodies and/or Lewy neurites with the presence or absence of alpha-synuclein aggregates including, but not limited to, Lewy bodies and/or Lewy neurites in the sample or specific body part or body area;
(v) optionally comparing the amount of the compound bound to the alpha-synuclein aggregates including, but not limited to, Lewy bodies and/or Lewy neurites to a normal control value; and
(vi) treating the patient with the medicament.

Optionally the method can further comprise step (A) after step (d) or step (e):
(A) comparing the amount of the compound bound to the alpha-synuclein aggregates including, but not limited to, Lewy bodies and/or Lewy neurites determined in step (iv) to the amount of the compound bound to the alpha-synuclein aggregates including, but not limited to, Lewy bodies and/or Lewy neurites determined in step (d).

In the method for predicting responsiveness the amount of the compound bound to the alpha-synuclein aggregates can be optionally compared at various points of time during the treatment, for instance, before and after onset of the treatment or at various points of time after the onset of the treatment. A change, especially a decrease, in the amount of the compound bound to the alpha-synuclein aggregates may indicate that the patient has a high potential of being responsive to the respective treatment.

If the amount of the compound bound to the alpha-synuclein aggregates decreases over time, it can be assumed that the patient is responsive to the treatment. If the amount of the compound bound to the alpha-synuclein aggregates is essentially constant or increases overtime, it can be assumed that the patient is non-responsive to the treatment.

Alternatively, the responsiveness can be estimated by determining the amount of the compound bound to the alpha-synuclein aggregates. The amount of the compound bound to the alpha-synuclein aggregates can be compared to a control value such as a normal control value, a preclinical control value or a clinical control value. Alternatively, the control value may refer to the control value of subjects known to be responsive to a certain therapy, or the control value may refer to the control value of subjects known to be non-responsive to a certain therapy. The outcome with respect to responsiveness can either be “responsive” to a certain therapy, “non-responsive” to a certain therapy or “response undetermined” to a certain therapy. Response to the therapy may be different for the respective patients.

Optionally, the diagnostic composition can be used before, during and after, surgical procedures (e.g. deep brain stimulation (DBS)) and non-invasive brain stimulation (such as repetitive transcranial magnetic stimulation (rTMS)), for visualizing alpha-synuclein aggregates before, during and after such procedures. Surgical techniques, including DBS, improve advanced symptoms of PD on top of the best currently used medical therapy. During the past 2 decades, rTMS has been closely examined as a possible treatment for PD (Ying-hui Chou et al. JAMA Neurol. 2015 Apr. 1; 72(4): 432-440).

In any of the above methods, the step of optionally correlating the presence or absence of the compound bound to the alpha-synuclein aggregates including, but not limited to, Lewy bodies and/or Lewy neurites with the presence or absence of the alpha-synuclein aggregates including, but not limited to, Lewy bodies and/or Lewy neurites in the sample or specific body part or body area; comprises
determining the amount of the compound bound to the alpha-synuclein aggregates including, but not limited to, Lewy bodies and/or Lewy;
correlating the amount of the compound bound to the alpha-synuclein aggregates including, but not limited to, Lewy bodies and/or Lewy neurites with the amount of the alpha-synuclein aggregates including, but not limited to, Lewy bodies and/or Lewy neurites in the sample or specific body part or body area; and
optionally comparing the amount of the compound bound with the alpha-synuclein aggregates including, but not limited to, Lewy bodies and/or Lewy neurites in the sample or specific body part or body area to a normal control value in a healthy control subject.

The control value can be, e.g., a normal control value, a preclinical control value and/or a clinical control value.

A “healthy control subject” or “healthy volunteer (HV) subject” is a person showing no clinical evidence of a disease, disorder or abnormality associated with alpha-synuclein aggregates including, but not limited to, Lewy bodies and/or Lewy neurites.

If in any of the above summarized methods the amount of the compound bound with the alpha-synuclein aggregates is higher than the normal control value, then it can be expected that the patient is suffering from or is likely to from a disease, disorder or abnormality associated with alpha-synuclein aggregates or from a synucleinopathy.

A sample or a specific body part or body area suspected to contain an alpha-synuclein aggregates, including but not limited to, Lewy bodies and/or Lewy neurites is brought into contact with a compound of the present invention.

Any of the compounds of the present invention can be used in the above summarized methods.

Preferably detectably labelled compounds of the present invention are employed in the above summarized methods.

The specific body part or body area is preferably of a mammal, more preferably of a human, including the full body or partial body area or body part of the patient suspected to contain alpha-synuclein aggregates. The specific body part or body area can be brain, the central nervous system, eye or a peripheral organ such as the gut, preferably brain.

The tissue can be brain tissue, tissue of the central nervous system (CNS), tissue of the eye (such as retinal tissue), tissue of peripheral organs such as the gut or other tissues, or body fluids such as cerebrospinal fluid (CSF) or blood. The tissue is preferably brain tissue. Preferably, the sample is an in vitro sample from a patient.

In the above methods, the compound of the present invention can be brought into contact with the sample or the specific body part or body area suspected to contain the alpha-synuclein aggregates including, but not limited to, Lewy bodies and/or Lewy neurites by any suitable method.

In in vitro methods the compound of the present invention and a liquid sample can be simply mixed.

In an in vivo method, the specific body part or body area can be brought into contact with a compound of the invention by administering an effective amount of a compound of the invention to the patient.

The effective amount of a compound of the invention is an amount which is suitable for allowing the presence or absence of alpha-synuclein aggregates including, but not limited to, Lewy bodies and/or Lewy neurites in the sample, specific body part or body area to be determined using the chosen analytical technique. The amount is not particularly limited and will depend on the compound of the formula (I), the type of detectable label, the sensitivity of the respective analytical method and the respective device. The amount can be chosen appropriately by a skilled person.

The compound is then allowed to bind to the alpha-synuclein aggregates, including but not limited to, Lewy bodies and/or Lewy neurites. The step of allowing the compound to bind to the alpha-synuclein aggregates including, but not limited to, Lewy bodies and/or Lewy neurites includes allowing sufficient time for the compound of the invention to bind to the alpha-synuclein aggregates including, but not limited to, Lewy bodies and/or Lewy neurites. The amount of time required for binding will depend on the type of test (e.g., in vitro or in vivo) and can be determined by a person skilled in the field by routine experiments. In an in vivo method, the amount of time will depend on the time which is required for the compound to reach the specific body part or body area suspected to contain alpha-synuclein aggregates including, but not limited to, Lewy bodies and/or Lewy neurites. The amount of time should not be too extended to avoid washout and/or metabolism of the compound of the invention.

The compound which has bound to the alpha-synuclein aggregates including, but not limited to, Lewy bodies and/or Lewy neurites, can be subsequently detected by any appropriate method. The method of detecting the compound bound to the alpha-synuclein aggregates including, but not limited to, Lewy bodies and/or Lewy neurites is not particularly limited and depends, among others, on the detectable label, the type of sample, specific body part or body area and whether the method is an in vitro or in vivo method. Examples of possible methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). These have been described and enable visualization of alpha-synuclein biomarkers. The fluorescence imaging technique and/or nuclear imaging technique can be employed for monitoring and/or visualizing the distribution of the detectably labelled compound within the sample or a specific body part or body area. The imaging system provides an image of bound detectable label such as radioisotopes, in particular positron emitters or gamma emitters, as present in the tested sample, the tested specific body part or the tested body area. Preferably, the compound bound to the alpha-synuclein aggregates including, but not limited to, Lewy bodies and/or Lewy neurites is detected by an imaging apparatus such as PET or SPECT scanner, more preferably PET.

The amount of the compound bound to the alpha-synuclein aggregates including, but not limited to, Lewy bodies and/or Lewy neurites can be determined by visual or quantitative analysis, for example, using PET scan images.

A compound according to the present invention or its precursor can also be incorporated into a test kit for detecting alpha-synuclein protein aggregates including, but not limited to, Lewy bodies and/or Lewy neurites. The test kit typically comprises a container holding one or more compounds according to the present invention or its precursor(s) and instructions for using the compound for the purpose of binding to alpha-synuclein aggregates including, but not limited to, Lewy bodies and/or Lewy neurites and detecting the formation of the compound bound to the alpha-synuclein aggregates such that presence or absence of the compound bound to the alpha-synuclein aggregates correlates with the presence or absence of the alpha-synuclein aggregates including, but not limited to, Lewy bodies and/or Lewy neurites.

The term “test kit” refers in general to any diagnostic kit known in the art. More specifically, the latter term refers to a diagnostic kit as described in Zrein et al., Clin. Diagn. Lab. Immunol., 1998, 5, 45-49.

The dose of the detectably labelled compounds of the present invention, preferably compounds of formula (I-F) labelled with ¹⁸F or compounds of formula (I-H*) or (I-H) labelled with ³H, will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

### Methods of Synthesizing the Compounds of the Invention

The compounds of the present invention may be prepared in accordance with the definition of compound of formula (I) by the routes described in the following Schemes or the Examples. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. In the following general methods, R¹, R², \textcircled{A}, X, LG, and n are as previously defined in the above embodiments, or limited to designations in the Schemes. Unless otherwise stated, starting materials are either commercially available or are prepared by known methods.

General Synthetic Scheme for the Preparation of Compounds and Precursors of this Invention:

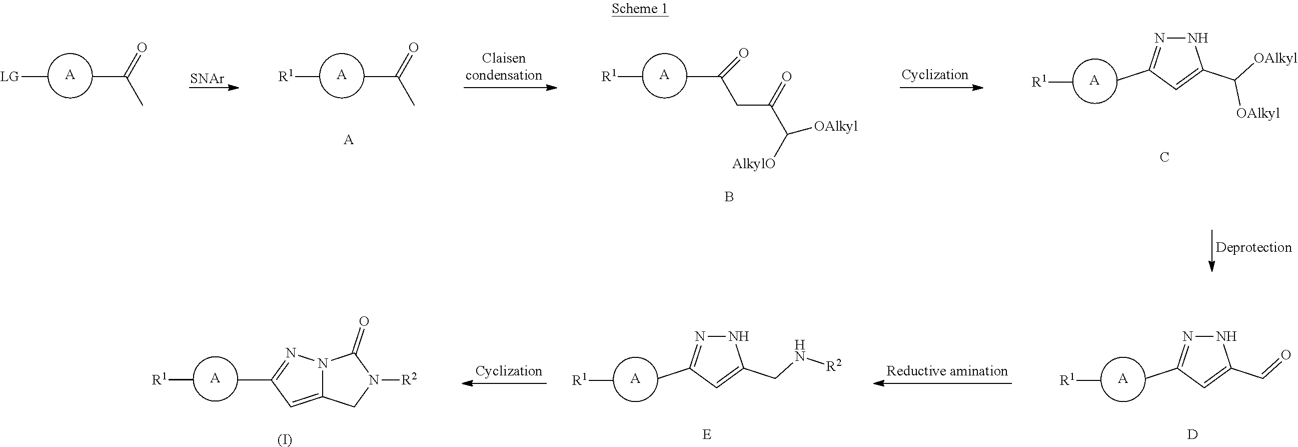

Commercially available ketone can be reacted with a nucleophile by a SNAr reaction to afford intermediate A. Claisen condensation with an appropriate ketone and ester can give intermediate B that can ring cyclized using hydrazine in an appropriate solvent. Deprotection of the acetal using acidic conditions can deliver the aldehyde D. Reductive amination with R²-amine and intermediate D in the presence of a reductive reagent can afford intermediate E. Finally, intermediate E can be ring cyclized using for example CDI in an appropriate solvent to give compounds of formula (I).

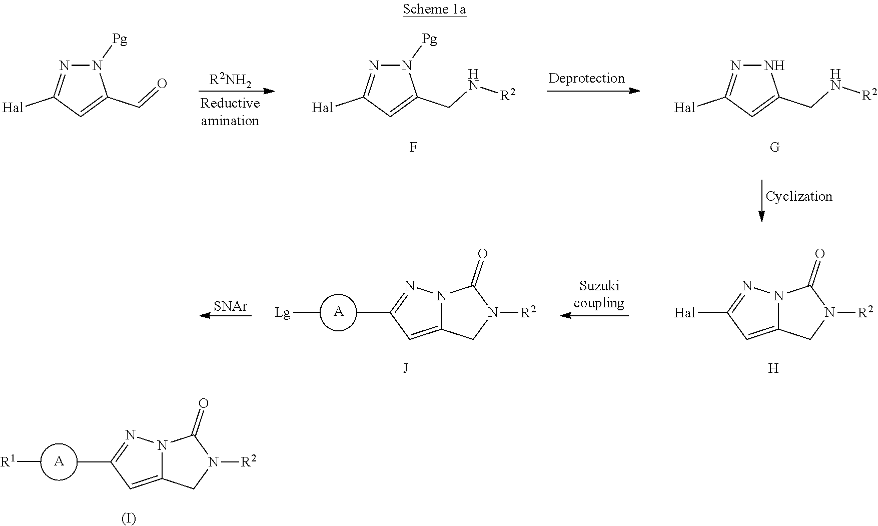

Reductive amination using commercially available aldehyde and appropriate amine can deliver amine intermediate F. Then, deprotection using adequate conditions can yield to NH pyrazole G. Subsequent ring cyclization, using for example CDI, can afford intermediate H. A ring can be introduced by Suzuki reaction using palladium source. Finally, intermediate J can be further functionalized using SNAr reaction with appropriate R¹ to give compounds of formula (I).

### General Synthesis of ¹⁸F-Labelled Compounds of the Present Invention

Compounds having the formula (I) which are labelled by ¹⁸F can be prepared by reacting a precursor compound, as described below, with an ¹⁸F-fluorinating agent, so that the LG comprised in the precursor compound is replaced by ¹⁸F.

The reagents, solvents and conditions which can be used for the ¹⁸F-fluorination are well-known to a skilled person in the field (L. Cai, S. Lu, V. Pike, Eur. J. Org. Chem 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile or DMSO.

Any suitable ¹⁸F-fluorinating agent can be employed. Typical examples include H¹⁸F, alkali or alkaline earth ¹⁸F-fluorides (e.g., K¹⁸F, Rb¹⁸F, Cs¹⁸F, and Na¹⁸F). Optionally, the ¹⁸F-fluorination agent can be used in combination with a chelating agent such as a cryptand (e.g.: 4,7,13,16,21,24-hexaoxa-1,10-diazabicyclo[8.8.8]-hexacosane—Kryptofix®) or a crown ether (e.g.: 18-crown-6). Alternatively, the ¹⁸F-fluorinating agent can be a tetraalkylammonium salt of ¹⁸F or a tetraalkylphosphonium salt of ¹⁸F; e.g., tetra(C₁₋₆ alkyl)ammonium salt of ¹⁸F or a tetra(C₁₋₆ alkyl)phosphonium salt of ¹⁸F. Preferably, the ¹⁸F-fluorination agent is K¹⁸F, H¹⁸F, Cs¹⁸F, Na¹⁸F, tetra(C₁₋₆ alkyl) ammonium salt of ¹⁸F, Kryptofix[222]¹⁸F or tetrabutylammonium [¹⁸F]fluoride.

Although the reaction is shown above with respect to ¹⁸F as a radioactive label, other radioactive labels can be introduced following similar procedures.

The invention is illustrated by the following examples which, however, should not be construed as limiting.

### EXAMPLES

### Exemplification of the Invention

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

Unless otherwise noted, all reagents and solvents were obtained from commercial sources and used without further purification.

The chemical names were generated using ChemBioDraw Ultra v20 from CambridgeSoft.

Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR.

### Abbreviations

Abbreviations used are those conventional in the art.

| | |
|---|---|
| CDI | carbonyldiimidazole |
| CsF | cesium fluoride |
| DCM | dichloromethane |
| DIEA | N,N-diisopropylethylamine |
| DMF | dimethylformamide |
| DMSO | dimethylsulfoxide |
| HPLC | High Performance Liquid Chromatography |
| LCMS | Liquid Chromatography Mass Spectrometry |
| LG | leaving group |
| Pg | protecting group |
| SNAr | nucleophilic aromatic substitution |
| STAB | sodium triacetoxyborohydride |
| THF | tetrahydrofuran |

### Analytical Details, Preparative and Analytical Methods

NMR measurements were performed on a DRX-400 MHz NMR spectrometer, on a Bruker AV-400 MHz NMR spectrometer or Spinsolve 80 MHz NMR spectrometer in deuterated solvents, using or not tetramethylsilane (TMS) as an internal standard. Chemical shifts (o) are reported in ppm downfield from TMS, spectra splitting patterns are designated as singlet (s), doublet (d), triplet (t), quartet (q), quintet (quint), septet (sept), multiplet, unresolved or overlapping signals (m), or broad signal (br). Deuterated solvents are given in parentheses and have chemical shifts of dimethyl sulfoxide (δ 2.50 ppm), methanol (δ 3.31 ppm), chloroform (δ 7.26 ppm), or other solvent as indicated in NMR spectral data.

Mass spectra (MS) were recorded on an Advion CMS mass spectrometer or an UPLC H-Class Plus with Photodiode Array detector and Qda Mass spectrometer from Waters.

Column chromatography was performed using silica gel (Fluka: Silica gel 60, 0.063-0.2 mm) and suitable solvents as indicated in the specific examples.

Flash Column Chromatography System: flash purification was conducted with a Biotage Isolera One flash purification system using HP-Sil or KP—NH SNAP cartridges (Biotage) and the solvent gradient indicated in the specific examples.

Thin layer chromatography (TLC) was carried out on silica gel plates with UV detection.

### Building Block Preparation

### Building Block Preparation 1:

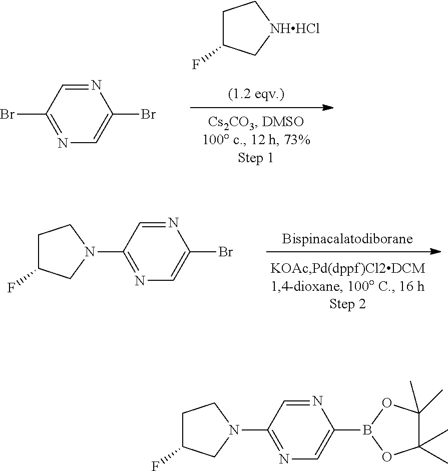

Step 1: In an oven-dried screw capped vial was added 2,5-dibromopyrazine (1.0 g, 4.2 mmol), (R)-3-fluoropyrrolidine hydrogen chloride (0.63 g, 5.1 mmol), Cs₂CO₃ (2.74 g, 8.4 mmol), and DMSO (10 mL) under an argon atmosphere. The mixture was heated to 100° C. for 12 h. Then, the reaction mixture was quenched with ice cold water (15 mL). The crude reaction mass was filtered through Büchner funnel. The obtained mass was washed with hexane (3×5 mL), dried under high vacuum to afford (R)-2-bromo-5-(3-fluoropyrrolidin-1-yl) pyrazine as off-white solid (0.76 g, 73%).

¹H NMR (DMSO-d6) δ 8.21 (d, 1H), 7.84 (d, 1H), 5.47 (dt, 1H), 3.71 (m, 2H), 3.60 (m, 1H), 3.44 (dd, 1H), 2.22 (m, 2H).

MS (ESI) 246.05 [M+H]+

Step 2:

In an oven-dried round-bottom flask was added (R)-2-bromo-5-(3-fluoropyrrolidin-1-yl) pyrazine (500 mg, 2.0 mmol), bispinacalatodiborane (620 mg, 2.4 mmol), KOAc (595 mg, 6.0 mmol) and 1,4-dioxane (15 mL) under an argon atmosphere. The reaction mixture was degassed with argon for 15 min. Then, Pd(dppf)Cl₂·DCM (165 mg, 0.2 mmol) was added and the mixture was heated to 100° C. for 16 h. After that the solvent was removed under high vacuum. To the obtained crude mass was added 40% EtOAc in hexane (3×40 mL x) and filtered through celite pad. The combined organic layer was concentrated under high vacuum. The obtained mass (R)-2-(3-fluoropyrrolidin-1-yl)-5-(4,4,5,5-tetramethyl-1,3,2-dioxa borolan-2-yl)pyrazine was directly used for next step without any further purification. MS (ESI) 294.26 [M+H]+

### Building Block Preparation 2:

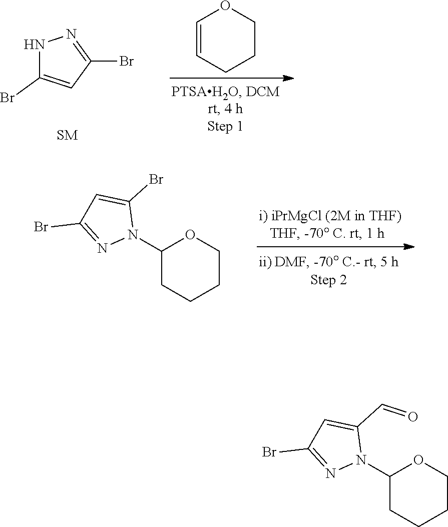 [SMILES: Brc1cc(Br)[nH]n1 ; C1=COCCC1 ; Brc1cc(Br)n(C2CCCCO2)n1 ; O=Cc1cc(Br)nn1C1CCCCO1]

Step 1: To a solution of 3,5-dibromo-1H-pyrazole (10 g, 44.4 mmol) in DCM (200 mL) were added 3,4-dihydro-2H-pyran (6.3 g, 75.5 mmol) and p-toluene sulfonic acid (PTSA) (0.5 g, 2.7 mmol). The reaction mixture was stirred at room temperature (RT) for 4 h. The progression of the reaction was monitored by TLC. After completion, the reaction was quenched with sat. aq. NaHCO₃ solution (2×60 mL x) and extracted with DCM (3×150 mL). The combined organic layer was dried over Na₂SO₄ and concentrated under vacuum. The obtained crude mass was purified by column chromatography over silica gel (230-400 mesh) eluted in 4% EtOAc in hexane to afford 3,5-dibromo-1-(tetrahydro-2H-pyran-2-yl)-1H-pyrazole as white solid (22 g, 80%).

¹H NMR (DMSO-d6) δ 6.76 (s, 1H), 5.44 (dd, 1H), 3.90 (m, 1H), 3.61 (m, 1H), 2.19 (m, 1H), 1.97 (m, 1H), 1.87 (qd, 1H), 1.69 (m, 1H), 1.51 (m, 2H).

MS (ESI) 309.85 [M+H]+

Step 2: To a solution of 3,5-dibromo-1-(tetrahydro-2H-pyran-2-yl)-1H-pyrazole (10 g, 32.3 mmol) in THF (350 mL) was added iPrMgCl (2M in THF, 21 mL, 42 mmol) dropwise with stirring at −70° C. under an argon atmosphere. During the addition, the temperature was kept below −60° C. The reaction mixture was stirred at −70° C./−60° C. for 1 h. Then, to the reaction mixture was added DMF (25 mL, 32.3 mmol) dropwise with stirring, keeping the temperature below −60° C. The reaction mixture was stirred for 5 min at the same temperature then gradually warmed up to room temperature and kept for 5 h. After completion, the reaction was quenched with saturated aqueous NH4Cl solution (80 mL) and extracted with EtOAc (3×150 mL). The combined organic layer was dried over Na₂SO₄ and concentrated under vacuum. The obtained crude mass was purified by column chromatography over silica gel (230-400 mesh) eluted in 40% EtOAc in hexane to afford 3-bromo-1-(tetrahydro-2H-pyran-2-yl)-1H-pyrazole-5-carbaldehyde as yellow solid (6.3 g, 75%).

¹H NMR (DMSO-d6) δ 9.93 (s, 1H), 7.24 (s, 1H), 6.03 (dd, 1H), 3.90 (m, 1H), 3.63 (m, 1H), 2.19 (m, 1H), 1.95 (m, 3H), 1.65 (m, 1H), 1.53 (m, 2H).

MS (ESI) 259.95 [M+H]+

### PREPARATIVE EXAMPLES

### Preparative Example 1

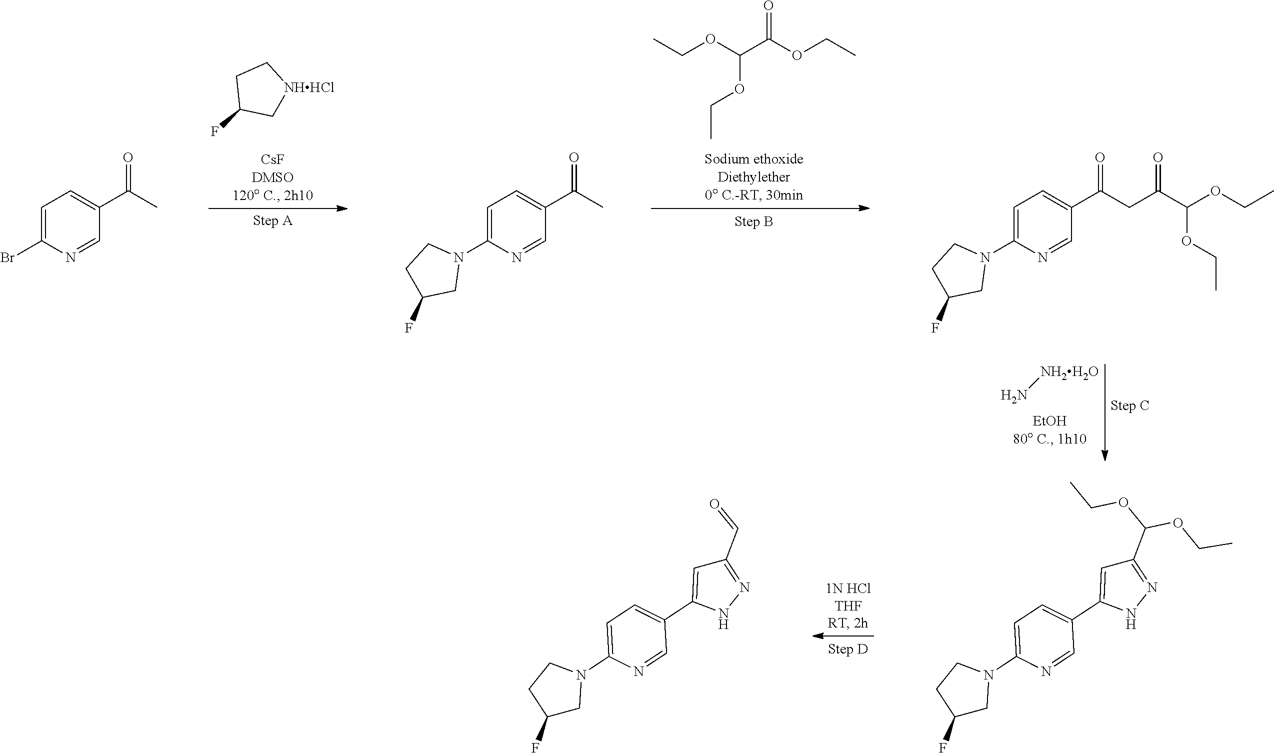

Step-A: In a flask, 1-(6-bromopyridin-3-yl)ethanone (2, 10.00 mmol), (S)-3-fluoropyrrolidine hydrochloride (2.51 g, 20.00 mmol) and cesium fluoride (9.11 g, 60.0 mmol) were heated at 120° C. in dry dimethylsulfoxide (40 mL). After 1 h 35 min, cesium fluoride (4.6 g, 30.0 mmol) was added and the mixture was stirred at 120° C. for an additional 35 minutes. Water was added and the product was extracted three times with dichloromethane. The combined organic layers were washed with water, dried over Na₂SO₄, filtered and concentrated under reduced pressure. The crude product was purified by flash chromatography (Silica 100 g column, 20-80% ethyl acetate in heptane) to afford (S)-1-(6-(3-fluoropyrrolidin-1-yl)pyridin-3-yl)ethenone as a light yellow solid (1.66 g, 80%)

¹H NMR (80 MHz, DMSO-d6) δ 8.73 (d, 1H), 7.99 (dd, 1H), 6.56 (d, 1H), 5.47 (d, 1H), 4.03-3.48 (m, 4H), 2.45 (s, 3H), 2.29-1.73 (m, 2H).

MS: 209.03 [M+H]⁺

Step B: In a flask under argon, the compound from step A (1.65 g, 7.92 mmol) and ethyl diethoxyacetate (4.27 mL, 23.77 mmol) were mixed in diethylether (60 mL). Sodium ethoxide (3.24 g, 47.5 mmol) was added at 0° C. and the mixture was stirred at room temperature for 30 minutes. The mixture was diluted with ethyl acetate, cooled in an ice bath and a 1 N aqueous HCl solution was added until pH 6-7 was reached. The mixture was diluted with water and the two layers separated. The organic layer was washed with brine, dried over Na₂SO₄, filtered and concentrated to dryness. The crude product was purified by flash chromatography (Silica 100 g column, 20-80% ethyl acetate in heptane) to afford (S)-1-(6-(3-fluoropyrrolidin-1-yl)pyridin-3-yl)ethenone as a brown-yellow solid (2.39 g, 89%)

¹H NMR (80 MHz, DMSO-d6) δ 8.72 (d, J=4.7, 2.3 Hz, 1H), 8.00 (dd, J=9.1, 5.2, 2.4 Hz, 1H), 6.78-6.41 (m, 2H), 5.35 (d, 1H), 4.86 (d, J=12.5 Hz, 1H), 4.22-3.16 (m, 8H), 2.28-1.82 (m, 2H), 1.15 (t, 6H).

MS: 339.11 [M+H]⁺

Step C: In a flask under argon, the compound from step B (2.39 g, 7.06 mmol) was dissolved in ethanol (70 mL). Hydrazine hydrate (0.756 mL, 7.77 mmol) was added dropwise and the reaction mixture was refluxed for 1 h 10 min. The solvent was evaporated, the crude product was dissolved in an aqueous solution of sodium bicarbonate and extracted twice with ethyl acetate. The organic layers were washed with brine, dried over Na₂SO₄, filtered and concentrated to dryness to afford (S)-5-(3-(diethoxymethyl)-1H-pyrazol-5-yl)-2-(3-fluoropyrrolidin-1-yl)pyridine as a white solid (2.08 g, 6.22 mmol).

¹H NMR (80 MHz, DMSO-d6) δ 12.91 (s, 1H), 8.50 (d, J=2.3 Hz, 1H), 7.89 (dd, J=8.8, 2.4 Hz, 1H), 6.73-6.42 (m, 2H), 5.89-4.97 (m, 2H), 3.95-3.42 (m, 8H), 2.37-1.59 (m, 2H), 1.15 (t, J=7.0 Hz, 6H).

MS: 335.14 [M+H]+

Step D: The compound from step C (2.08 g, 6.22 mmol) was dissolved in tetrahydrofuran (50 mL) and an aqueous solution of 1N hydrochloric acid (15 mL, 494 mmol) was added. The reaction mixture was stirred at room temperature for 1 h 20 min. An additional aqueous solution of 1N hydrochloric acid (10 mL, 329 mmol) was added and the reaction mixture was stirred at room temperature for an extra 40 minutes. The mixture was basified to pH 14 with an aqueous solution of 1N sodium hydroxide. Ethyl acetate was added and the aqueous phase was extracted twice. The organic layers were combined and washed with a saturated solution of NaHCO₃ and brine. The organic layer was concentrated to afford (S)-5-(6-(3-fluoropyrrolidin-1-yl)pyridin-3-yl)-1H-pyrazole-3-carbaldehyde as a white solid (1.66 g, 6.38 mmol).

¹H NMR (80 MHz, DMSO-d6) δ 9.90 (s, 1H), 8.58 (d, J=2.4 Hz, 1H), 7.95 (dd, J=8.8, 2.4 Hz, 1H), 7.12 (s, 1H), 6.60 (d, J=8.8 Hz, 1H), 5.46 (d, J=53.4 Hz, 1H), 4.12-3.52 (m, 4H), 2.22-1.72 (m, 2H).

MS: 261.05 [M+H]⁺

### Preparative Example 2

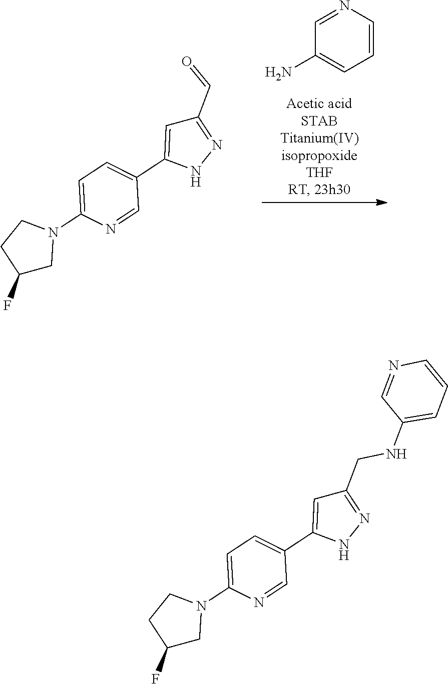 [SMILES: O=Cc1cc(-c2ccc(N3CC[C@H](F)C3)nc2)[nH]n1 ; Nc1cccnc1 ; F[C@H]1CCN(c2ccc(-c3cc(CNc4cccnc4)n[nH]3)cn2)C1]

To a solution of the compound from Preparative Example 1 (250 mg, 0.961 mmol) in tetrahydrofuran (15 mL) at room temperature was added titanium (IV) isopropoxide (0.141 mL, 0.480 mmol) and the mixture was stirred for 5 minutes. 3-Aminopyridine (181 mg, 1.921 mmol) and acetic acid (6 mL) were added and the mixture was stirred at room temperature until full conversion to the imine (20 h). During that time, 6 mL of acetic acid were added. Sodium triacetoxyborohydride (1425 mg, 6.72 mmol) was added and the mixture was stirred for 3 h. The reaction mixture was quenched with an aqueous solution of sodium hydroxide 1N to reach pH 14. The aqueous layer was extracted twice with ethyl acetate. The organic layers were combined, washed twice with a solution of 1 N NaOH, once with brine, dried over Na₂SO₄, filtered and concentrated to dryness. The crude product was suspended in dichloromethane, stirred at reflux and hot-filtrated. The same process was conducted with ethanol. All the product went into the filtrate, and the latter was concentrated to dryness to afford (S)—N-((5-(6-(3-fluoropyrrolidin-1-yl)pyridin-3-yl)-1H-pyrazol-3-yl)methyl)pyridin-3-amine as a salmon-colored solid (70.1 mg, 0.207 mmol).

¹H NMR (80 MHz, DMSO-d6) δ 12.80 (s, 1H), 8.46 (d, J=2.2 Hz, 1H), 8.11-7.66 (m, 3H), 7.20-6.87 (m, 2H), 6.66-6.40 (m, 2H), 6.28 (t, J=5.8 Hz, 1H), 5.44 (d, J=53.3 Hz, 1H), 4.24 (d, J=5.8 Hz, 2H), 3.93-3.48 (m, 4H), 2.24-1.88 (m, 2H).

¹⁹F NMR (76 MHz, DMSO-d6) δ −69.70.

MS: 339.11 [M+H]⁺

### Preparative Example 3

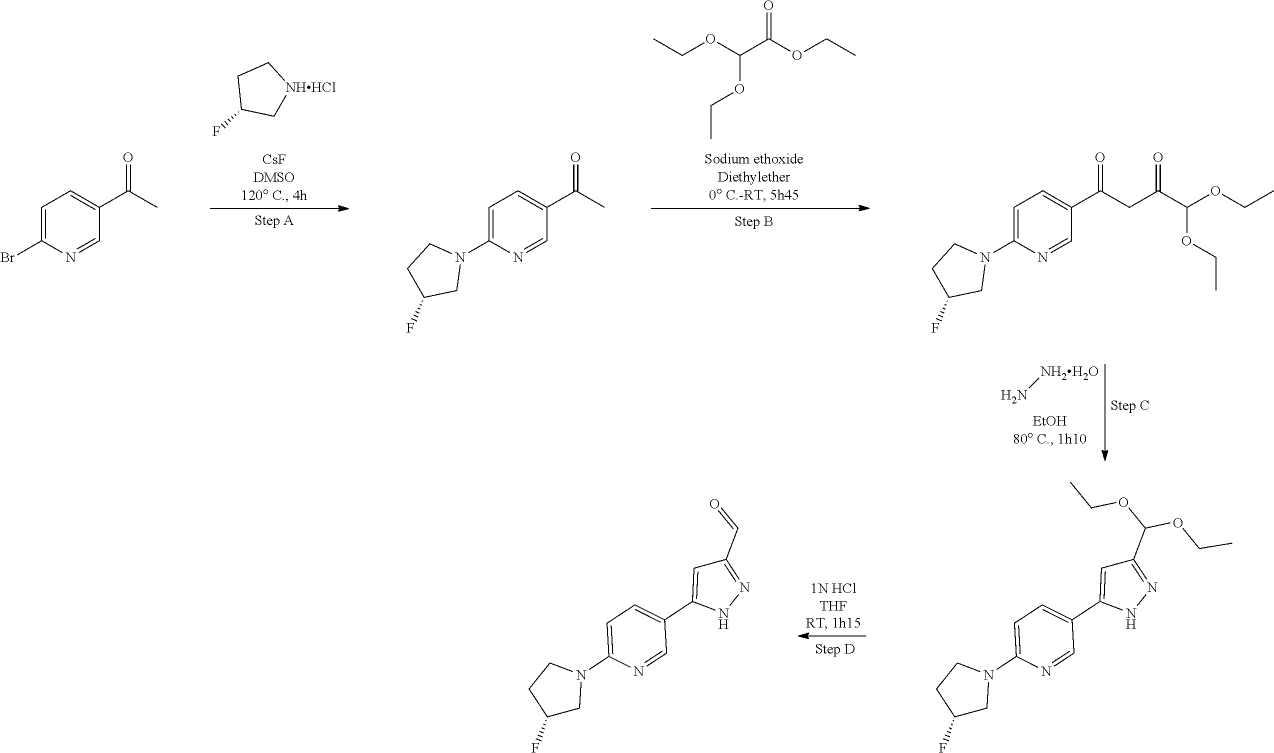

Step A: In a vial under argon, 1-(6-bromopyridin-3-yl)ethanone (2.5 g, 12.50 mmol), (R)-3-fluoropyrrolidine hydrochloride (3.14 g, 25.00 mmol), and cesium fluoride (5.70 g, 37.5 mmol) were mixed in dry dimethylsulfoxide (40 mL). The mixture was flushed with argon and stirred at 120° C. for 1 h 30 min. Cesium fluoride (2.9, 18.8 mmol) was added and the mixture was stirred at 120° C. for an additional 30 minutes. The process was repeated another time. Water was added and the product was extracted six times with DCM. The crude product was purified by flash chromatography (Silica 100 g column, 0-5% methanol in dichloromethane) to afford (R)-1-(6-(3-fluoropyrrolidin-1-yl)pyridin-3-yl)ethenone as a brown oil (2.40 g, 11.53 mmol).

¹H NMR (80 MHz, DMSO-d6) δ 8.72 (d, 1H), 7.98 (dd, 1H), 6.55 (d, 1H), 5.56 (d, 1H), 4.04-3.39 (m, 4H), 2.45 (s, 3H), 2.38-1.78 (m, 2H).

MS: 209.05 [M+H]⁺

Step B: In a flask under argon, the compound from step A (2.40 g, 11.53 mmol) and ethyl diethoxyacetate (2.072 mL, 11.53 mmol) were mixed in diethyl ether (70 mL). Sodium ethoxide (1.569 g, 23.05 mmol) was added at 0° C. and the mixture was stirred at room temperature for 19 h. Ethyl diethoxyacetate (2.072 mL, 11.53 mmol) and sodium ethoxide (1.569 g, 23.05 mmol) were added at 0° C. After 2 h, the conversion was not complete; ethyl diethoxyacetate (1 mL, 5.56 mmol) and sodium ethoxide (1.569 g, 23.05 mmol) were added. The mixture was diluted with ethyl acetate, cooled in an ice bath and 1N aqueous HCl solution (25 mL) was added until pH 6-7 was reached. The mixture was diluted with water and the two layers separated. The organic layer was washed with brine, dried over Na₂SO₄, filtered and concentrated to dryness. The crude product was purified by flash chromatography (Silica 100 g column, 0-10% methanol in dichloromethane), and re-purified by flash chromatography (Silica 100 g column, 20-80% ethyl acetate in heptane) to afford (R)-4,4-diethoxy-1-(6-(3-fluoropyrrolidin-1-yl)pyridin-3-yl)butane-1,3-dione as a yellow solid (1.61 g, 4.76 mmol).

¹H NMR (80 MHz, DMSO-d6) δ 8.72 (d, 1H), 8.00 (dd, 1H), 6.74-6.44 (m, 2H), 5.77 (d, 1H), 4.86 (d, 1H), 4.18-3.09 (m, 8H), 2.37-1.79 (m, 2H), 1.16 (t, 6H).

MS: 339.12 [M+H]⁺

Step C: In a flask under argon, the compound from step B (1.61 g, 4.76 mmol) was dissolved in ethanol (65 mL). Hydrazine hydrate (0.509 mL, 5.23 mmol) was added dropwise and the reaction mixture was refluxed for 1 h 10 min. The solvent was evaporated, the crude product was dissolved in an aqueous solution of sodium bicarbonate and extracted twice with ethyl acetate. The organic layers were washed with brine, dried over Na₂SO₄, filtered and concentrated to dryness to afford (R)-5-(3-(diethoxymethyl)-1H-pyrazol-5-yl)-2-(3-fluoropyrrolidin-1-yl)pyridine as a white solid (1.37 g, 4.10 mmol).

¹H NMR (80 MHz, DMSO-d6) δ 12.94 (s, 1H), 8.50 (d, 1H), 7.89 (dd, 1H), 6.66-6.42 (m, 2H), 5.86-5.04 (m, 2H), 3.94-3.42 (m, 8H), 2.22-1.58 (m, 2H), 1.15 (t, 6H).

MS: 335.12 [M+H]⁺

Step D: The compound from step C (1.37 g, 4.10 mmol) was dissolved in tetrahydrofuran (40 mL) and an aqueous solution of 1 N hydrochloric acid (10 ml, 329 mmol) was added. The reaction mixture was stirred at room temperature for 1 h 15 min. The mixture was basified to pH 14 with an aqueous solution of 1N sodium hydroxide. Ethyl acetate and a saturated solution of NaHCO₃ were added and the layers separated. The aqueous phase was extracted twice, the organic layers were combined and washed once with brine. The organic layer was concentrated to afford (R)-5-(6-(3-fluoropyrrolidin-1-yl)pyridin-3-yl)-1H-pyrazole-3-carbaldehyde as a beige solid (912 mg, 3.50 mmol).

¹H NMR (80 MHz, DMSO-d6) δ 9.90 (s, 1H), 8.58 (d, 1H), 7.95 (dd, 1H), 7.12 (s, 1H), 6.60 (d, 1H), 5.46 (d, 1H), 3.94-3.52 (m, 4H), 2.26-1.78 (m, 2H).

MS: 261.03 [M+H]⁺

### Preparative Example 4

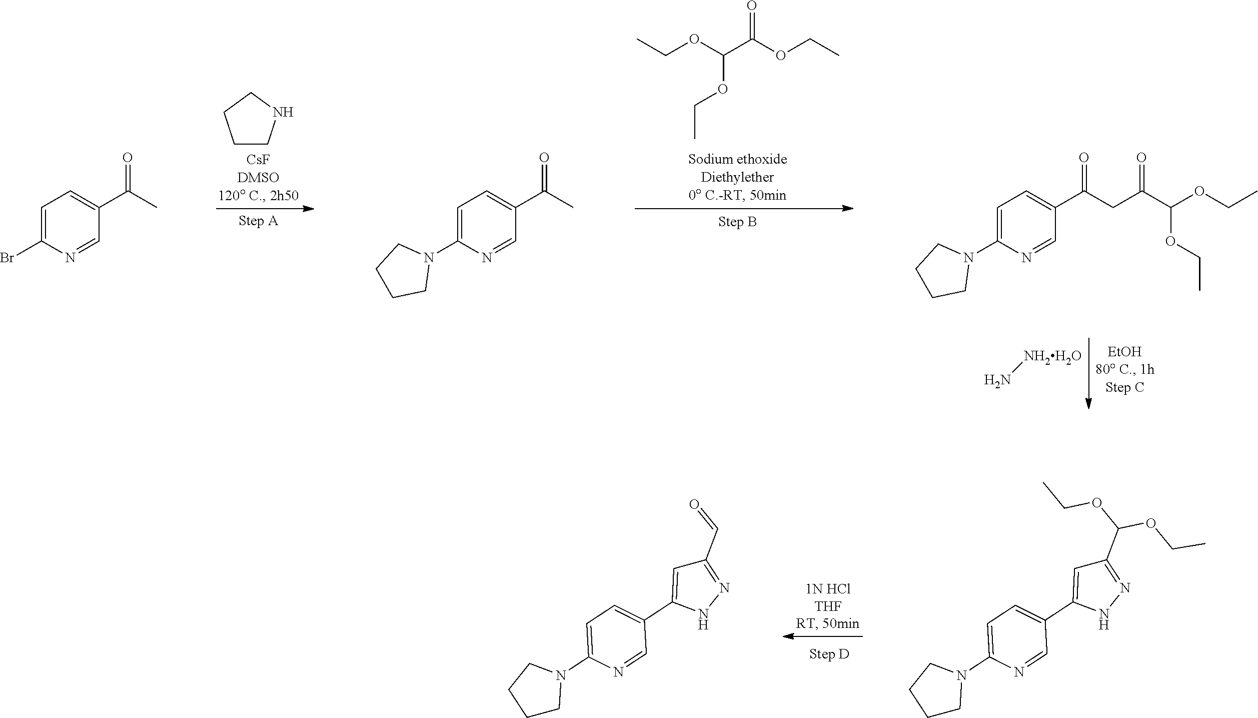

Step-A: In a flask, 1-(6-bromopyridin-3-yl)ethanone (2 g, 10.00 mmol), pyrrolidine (2.504 ml, 30.0 mmol), and cesium fluoride (9.11 g, 60.0 mmol) were mixed in dry dimethylsulfoxide (60 mL). The mixture was stirred at 120° C. for 2 h 50 min. Water was added and the product was extracted twice with dichloromethane. The organic layer was washed with water three times, dried over Na₂SO₄, filtered and concentrated to dryness to afford 1-(6-(pyrrolidin-1-yl)pyridin-3-yl)ethenone as an orange solid (1.84 g, 9.67 mmol).

¹H NMR (80 MHz, DMSO-d6) δ 8.71 (d, 1H), 7.94 (dd, 1H), 6.48 (d, 1H), 3.64-3.36 (m, 4H), 2.43 (s, 3H), 2.15-1.76 (m, 4H).

MS: 191.04 [M+H]⁺

Step-B: In a flask under argon, the compound from step A (1.84 g, 9.67 mmol) and ethyl diethoxyacetate (5.22 mL, 29.0 mmol) were mixed in diethyl ether (80 mL). Sodium ethoxide (3.95 g, 58.0 mmol) was added at 0° C. and the mixture was stirred at room temperature for 50 min. The mixture was diluted with ethyl acetate, cooled in an ice bath and 1N aqueous HCl solution was added until pH 6-7 was reached. The mixture was diluted with water and the two layers separated. The aqueous phase was extracted once. The organic layers were combined, washed with brine, dried over Na₂SO₄, filtered and concentrated to dryness. The crude product was purified by flash chromatography (Silica 100 g column, 20-80% ethyl acetate in heptane) to afford 4,4-diethoxy-1-(6-(pyrrolidin-1-yl)pyridin-3-yl)butane-1,3-dione as a yellow oil (3.1 g, 9.68 mmol).

¹H NMR (80 MHz, DMSO-d6) δ 8.71 (d, 1H), 7.95 (dd, 1H), 6.66-6.38 (m, 2H), 5.01-4.73 (m, 2H), 4.11 (s, 1H), 3.80-3.28 (m, 8H), 2.10-1.78 (m, 4H), 1.32-0.97 (m, 6H).

MS: 321.09 [M+H]⁺

Step-C: In a flask under argon, the compound from step B (3.1 g, 9.68 mmol) was dissolved in ethanol (80 mL). Hydrazine hydrate (1.036 mL, 10.64 mmol) was added dropwise and the reaction mixture was refluxed for 1 h. The solvent was evaporated, the crude product was dissolved in an aqueous solution of sodium bicarbonate and extracted twice with ethyl acetate. The organic layers were washed with brine, dried over Na₂SO₄, filtered and concentrated to dryness to afford 5-(3-(diethoxymethyl)-1H-pyrazol-5-yl)-2-(pyrrolidin-1-yl)pyridine as a light yellow solid (1.88 g, 5.94 mmol).

¹H NMR (80 MHz, DMSO-d6) δ 12.84 (s, 1H), 8.47 (d, 1H), 7.84 (dd, 1H), 6.66-6.30 (m, 2H), 5.52 (s, 1H), 3.80-3.34 (m, 8H), 2.08-1.78 (m, 4H), 1.15 (t, J=7.0 Hz, 6H).

MS: 317.12 [M+H]⁺

Step-D: The compound from step C (1.88 g, 5.94 mmol) was dissolved in tetrahydrofuran (50 mL) and hydrochloric acid 1N aqueous solution (25 ml, 823 mmol) was added. The reaction mixture was stirred at room temperature for 50 minutes. The mixture was basified to pH 14 with an aqueous solution of 1N NaOH. Ethyl acetate was added and the aqueous phase was extracted twice. The organic layer was washed with a saturated solution of NaHCO₃, followed by brine. The organic layer was concentrated to afford 5-(6-(pyrrolidin-1-yl)pyridin-3-yl)-1H-pyrazole-3-carbaldehyde as a light yellow solid (876 mg, 3.62 mmol).

¹H NMR (80 MHz, DMSO-d6) δ 13.95 (s, 1H), 9.89 (s, 1H), 8.55 (d, 1H), 7.90 (dd, 1H), 7.08 (s, 1H), 6.53 (d, 1H), 3.56-3.36 (m, 4H), 2.08-1.83 (m, 4H).

MS: 243.06 [M+H]⁺

### Preparative Example 5

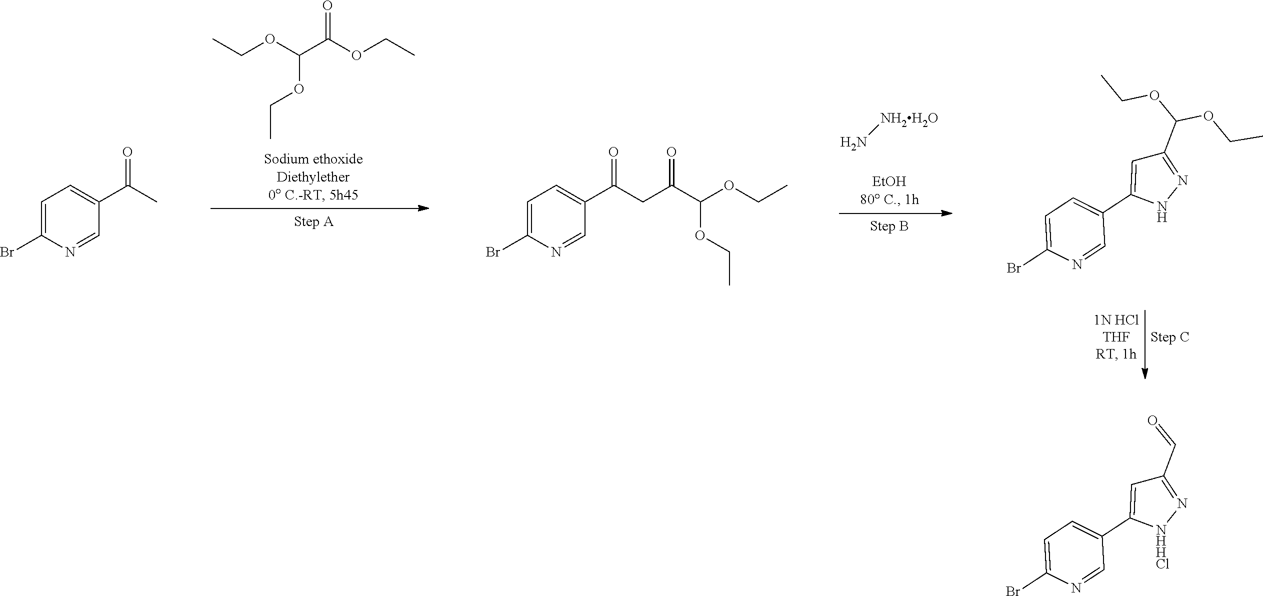

Step-A: In a flask under argon, 1-(6-bromopyridin-3-yl)ethanone (2 g, 10.00 mmol) and ethyl diethoxyacetate (1.798 mL, 10.00 mmol) were mixed in diethyl ether (50 mL). Sodium ethoxide (0.680 g, 10.00 mmol) was added at 0° C. and the mixture was stirred at room temperature for 3 h 15 min. The mixture was then refluxed for 1 h before addition of sodium ethoxide (0.680 g, 10.00 mmol). The reaction mixture was further stirred for 1 h 30 min before completion. The mixture was diluted with ethyl acetate, cooled in an ice bath and 1N aqueous HCl solution (25 mL) was added until pH 6-7 was reached. The mixture was diluted with water and the two layers separated. The organic layer was washed with brine, dried over Na₂SO₄, filtered and concentrated to dryness. The crude product was purified by flash chromatography (Silica 100 g column, 5-40% ethyl acetate in heptane) to afford 1-(6-bromopyridin-3-yl)-4,4-diethoxybutane-1,3-dione as a white solid (1.175 g, 3.56 mmol).

¹H NMR (80 MHz, DMSO-d6) δ 8.93 (d, 1H), 8.22 (dd, 1H), 7.84 (d, 1H), 6.70 (s, 1H), 4.90 (d, 1H), 4.33 (s, 1H), 3.64 (q, 4H), 1.18 (t, 6H).

MS: 331.98 [M+H]⁺

Step-B: In a flask under argon, the compound from step A (1.18 g, 3.57 mmol) was dissolved in ethanol (50 mL). Hydrazine hydrate (0.383 mL, 3.93 mmol) was added dropwise and the reaction mixture was refluxed for 1 h. The solvent was evaporated, the crude product was dissolved in an aqueous solution of sodium bicarbonate and extracted twice with ethyl acetate. The organic layers were washed with brine, dried over Na₂SO₄, filtered and concentrated to dryness to afford 2-bromo-5-(3-(diethoxymethyl)-1H-pyrazol-5-yl)pyridine as a white solid (1.17 g, 3.59 mmol).

¹H NMR (80 MHz, DMSO-d6) δ 13.19 (s, 1H), 8.82 (d, 1H), 8.13 (dd, 1H), 7.67 (d, 1H), 6.82 (s, 1H), 5.67 (s, 1H), 3.58 (q, 4H), 1.16 (t, 6H).

MS: 326.00 [M+H]⁺

Step C: The compound from step B (1.17 g, 3.59 mmol) was dissolved in tetrahydrofuran (40 mL) and hydrochloric acid 1N solution (10 ml, 329 mmol) was added. The reaction mixture was stirred at room temperature for 1 h. The mixture was basified to pH 9 with an aqueous solution of 1N NaOH. Ethyl acetate and a saturated solution of NaHCO₃ were added and the layers separated. The product precipitated in the organic phase, and it was filtered to afford 5-(6-bromopyridin-3-yl)-1H-pyrazole-3-carbaldehyde hydrochloride as a white solid (989.1 mg, 3.92 mmol).

¹H NMR (80 MHz, DMSO-d6) δ 9.87 (s, 1H), 8.86 (d, 1H), 8.16 (dd, 1H), 7.67 (d, 1H), 7.28 (s, 1H).

MS: 253.95 [M+H]⁺

### Preparative Examples 6 to 14

Following the reductive reaction procedure as described in Preparative Example 2, using the aldehyde amine starting material and the reducing agent indicated in Table 1 below, the following Preparative Examples were prepared.

TABLE 1

| | | | | 1. Yield |
|---|---|---|---|---|
| | Reducing | | | 2. ¹H-NMR |
| Amine | agent | Aldehyde | Preparative example | 3. MH⁺ (ESI) |
| 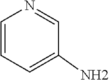 | STAB | 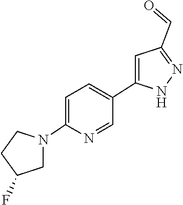 [SMILES: O=Cc1cc(-c2ccc(N3CC[C@@H](F)C3)nc2)[nH]n1] | 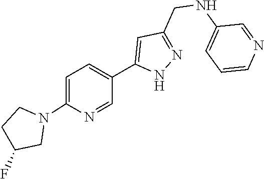 [SMILES: F[C@@H]1CCN(c2ccc(-c3cc(CNc4cccnc4)n[nH]3)cn2)C1] | 1.7% 2. ¹H NMR (80 MHz, DMSO-d6) δ 12.78 (s, 1H), 8.55-8.35 (m, 1H), 8.12- 7.65 (m, 3H), 7.19-6.84 (m, 2H), 6.60 (d, 1H), 6.49 (s, 1H), 6.27 (t, 1H), 5.44 (d, 1H), 4.25 (d, 2H), 3.98- 3.49 (m, 4H), 2.21-1.82 (m, 2H). 3. 339.08 |
| | | | 6 | |
| 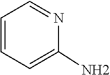 | STAB | 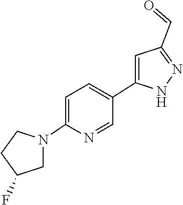 [SMILES: O=Cc1cc(-c2ccc(N3CC[C@@H](F)C3)nc2)[nH]n1] | 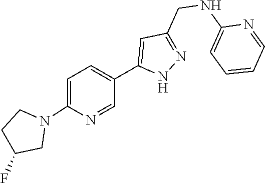 [SMILES: F[C@@H]1CCN(c2ccc(-c3cc(CNc4ccccn4)n[nH]3)cn2)C1] | 1. 16% 2. ¹H NMR (80 MHz, DMSO-d6) δ 12.68 (s, 1H), 8.46 (d, 1H), 8.10-7.73 (m, 2H), 7.38 (t, 1H), 6.80 (s, 1H), 6.64-6.35 (m, 4H), 5.44 (d, 1H), 4.43 (d, 2H), 3.97-3.49 (m, 4H), 2.22- 1.75 (m, 2H). 3. 339.06 |
| | | | 7 | |
| 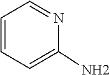 | NaCNBH₃ | 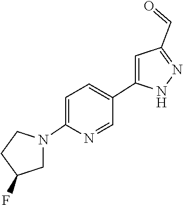 [SMILES: O=Cc1cc(-c2ccc(N3CC[C@H](F)C3)nc2)[nH]n1] | 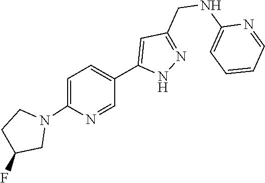 [SMILES: F[C@H]1CCN(c2ccc(-c3cc(CNc4ccccn4)n[nH]3)cn2)C1] | 1. 15% 2. ¹H NMR (80 MHz, DMSO-d6) δ 12.62 (s, 1H), 8.49 (d, 1H), 8.12-7.74 (m, 2H), 7.42 (t, 1H), 6.86 (s, 1H), 6.67-6.36 (m, 4H), 5.47 (d, 1H), 4.47 (d, 2H), 4.00-3.60 (m, 4H), 2.26- 1.79 (m, 2H). 3. 339.10 |
| | | | 8 | |
| 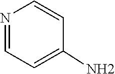 | NaCNBH₃ | 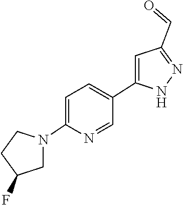 [SMILES: O=Cc1cc(-c2ccc(N3CC[C@H](F)C3)nc2)[nH]n1] | 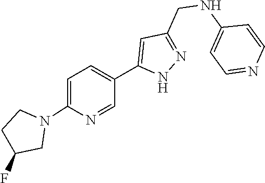 [SMILES: F[C@H]1CCN(c2ccc(-c3cc(CNc4ccncc4)n[nH]3)cn2)C1] | 1. 16% 2. ¹H NMR (80 MHz, DMSO-d6) δ 12.53 (s, 1H), 8.47 (d, 1H), 8.03 (d, 2H), 7.85 (dd, 1H), 7.02 (t, 1H), 6.68-6.38 (m, 4H), 5.45 (d, 1H), 4.27 (d, 2H), 3.92 3.49 (m, 4H), 2.23-1.87 (m, 2H). 3. 339.11 |
| | | | 9 | |
| 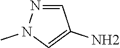 | NaCNBH₃ | 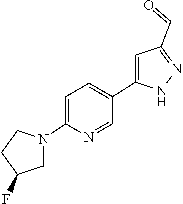 [SMILES: O=Cc1cc(-c2ccc(N3CC[C@H](F)C3)nc2)[nH]n1] | 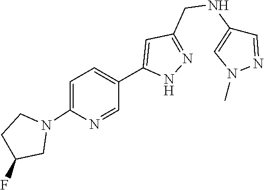 [SMILES: Cn1cc(NCc2cc(-c3ccc(N4CC[C@H](F)C4)nc3)[nH]n2)cn1] | 1. 39% 2. ¹H NMR (80 MHz, DMSO-d6) δ 12.66 (s, 1H), 8.46 (d, 1H), 7.85 (dd, 1H), 7.07 (s, 1H), 6.97 (s, 1H), 6.63-6.43 (m, 2H), 5.45 (d, 1H), 4.70 (t, 1H), 4.00 (d, 2H), 3.93-3.38 (m, 7H), 2.23-1.90 (m, 2H). 3. 342.11 |
| | | | 10 | |
| 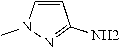 | NaCNBH₃ | 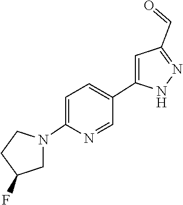 [SMILES: O=Cc1cc(-c2ccc(N3CC[C@H](F)C3)nc2)[nH]n1] | 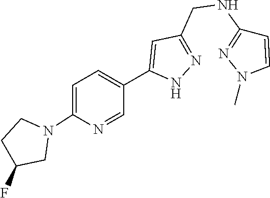 [SMILES: Cn1ccc(NCc2cc(-c3ccc(N4CC[C@H](F)C4)nc3)[nH]n2)n1] | 1. 57% 2. ¹H NMR (80 MHz, DMSO-d6) δ 12.62 (s, 1H), 8.45 (d, 1H), 7.84 (dd, 1H), 7.30 (d, 1H), 6.65-6.38 (m, 2H), 5.45 (d, 1H), 5.44 (d, 1H), 4.17 (d, 2H), 3.92- 3.42 (m, 7H), 2.24-1.89 (m, 2H). 3. 342.11 |
| | | | 11 | |
| 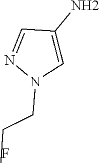 | NaCNBH₃ | 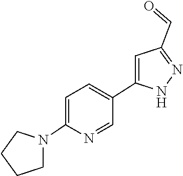 [SMILES: O=Cc1cc(-c2ccc(N3CCCC3)nc2)[nH]n1] | 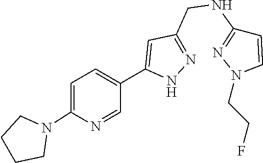 [SMILES: FCCn1ccc(NCc2cc(-c3ccc(N4CCCC4)nc3)[nH]n2)n1] | 1. 14% 2. ¹H NMR (80 MHz, DMSO-d6) δ 12.61 (s, 1H), 8.43 (d, 1H), 7.80 (dd, 1H), 7.16 (s, 1H), 7.05 (s, 1H), 6.58-6.39 (m, 2H), 4.52- 4.29 (m, 2H), 4.19-3.87 (m, 4H), 3.53-3.38 (m, 4H), 2.06-1.81 (m, 4H). 3. 356.15 |
| | | | 12 | |
| 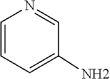 | STAB | 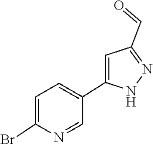 [SMILES: O=Cc1cc(-c2ccc(Br)nc2)[nH]n1] | 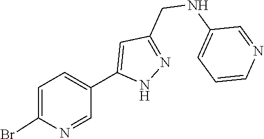 [SMILES: Brc1ccc(-c2cc(CNc3cccnc3)n[nH]2)cn1] | 1. 18% 2. ¹H NMR 13.11 (s, 1H), 8.77 (d, 1H), 8.18-7.97 (m, 2H), 7.84-7.59 (m, 2H), 7.11-6.93 (m, 2H), 6.77 (s, 1H), 6.34 (t, 1H), 4.32 (d, 2H). 3. 331.98 |
| | | | 13 | |
| 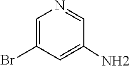 | NaCNBH₃ | 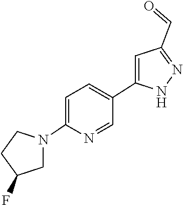 [SMILES: O=Cc1cc(-c2ccc(N3CC[C@H](F)C3)nc2)[nH]n1] | 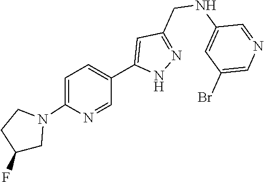 [SMILES: F[C@H]1CCN(c2ccc(-c3cc(CNc4cncc(Br)c4)n[nH]3)cn2)C1] | 1. 28% 2. ¹H NMR δ 12.92 (s, 1H), 8.46 (d, 1H), 8.01 (d, 1H), 7.96-7.67 (m, 2H), 7.21 (s, 1H), 6.80-6.39 (m, 3H), 5.45 (d, 1H), 4.29 (d, 2H), 3.96-3.46 (m, 4H), 2.18- 1.79 (m, 2H). 3. 419.05 |
| | | | 14 | |

### EXAMPLES

### Example 1

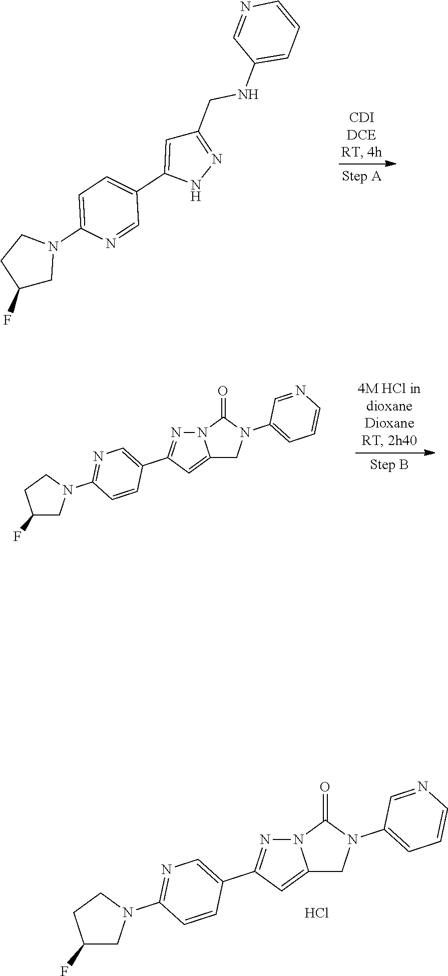 [SMILES: F[C@H]1CCN(c2ccc(-c3cc(CNc4cccnc4)n[nH]3)cn2)C1 ; O=C1N(c2cccnc2)Cc2cc(-c3ccc(N4CC[C@H](F)C4)nc3)nn21 ; O=C1N(c2cccnc2)Cc2cc(-c3ccc(N4CC[C@H](F)C4)nc3)nn21 ; Cl]

Step A: To a solution of the compound from Preparative Example 2 (65.1 mg, 0.192 mmol) in dichloroethane (6 mL) at room temperature was added 1,1′-carbonyldiimidazole (312 mg, 1.924 mmol). The mixture was stirred at room temperature for 4 h. The crude reaction mixture was filtrated and rinsed with a small amount of cold dichloroethane to afford (S)-2-(6-(3-fluoropyrrolidin-1-yl)pyridin-3-yl)-5-(pyridin-3-yl)-4H-imidazo[1,5-b]pyrazol-6(5H)-one as a pale rose solid (50.3 mg, 0.138 mmol).

¹H NMR (400 MHz, DMSO-d6) δ 8.97 (d, 1H), 8.67 (d, 1H), 8.42 (d, 1H), 8.19 (d, 1H), 8.05 (dd, 1H), 7.51 (dd, 1H), 6.93 (s, 1H), 6.62 (d, 1H), 5.47 (d, 1H), 5.15 (s, 2H), 3.95-3.54 (m, 4H), 2.35-2.11 (m, 2H).

¹⁹F NMR (76 MHz, DMSO-d6) δ −69.68.

MS: 365.12 [M+H]⁺

Step B: To a solution of the compound from step A (19 mg, 0.052 mmol) in dioxane (3 mL) at room temperature was added 4M HCl in dioxane (0.025 ml, 0.1 mmol). The mixture was stirred at room temperature for 2 h 40 min. The solvent was evaporated to afford (S)-2-(6-(3-fluoropyrrolidin-1-yl)pyridin-3-yl)-5-(pyridin-3-yl)-4H-imidazo[1,5-b]pyrazol-6(5H)-one hydrochloride as a pale rose solid (23.3 mg, 0.058 mmol).

¹H NMR (80 MHz, DMSO-d6) δ 9.04 (d, 1H), 8.63-8.21 (m, 4H), 7.64 (dd, 1H), 7.16-6.89 (m, 2H), 5.93-5.12 (m, 3H), 4.08-3.67 (m, 4H), 2.28-1.86 (m, 2H).

¹⁹F NMR (76 MHz, DMSO-d6) δ-69.58.

MS: 365.20 [M+H]⁺

### Examples 2 to 9

Following the cyclization reaction procedure as described in Example 1, using the material indicated in Table 2 below, the following Examples were prepared.

TABLE 2

| | | 1. Yield (over two steps) |
|---|---|---|
| | | 2. ¹H-NMR |
| Starting material | Example | 3. MH⁺ (ESI) |
| 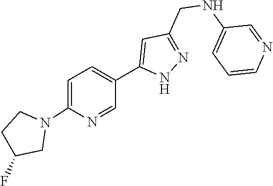 [SMILES: F[C@@H]1CCN(c2ccc(-c3cc(CNc4cccnc4)n[nH]3)cn2)C1] | 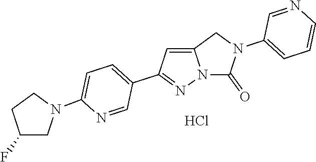 [SMILES: O=C1N(c2cccnc2)Cc2cc(-c3ccc(N4CC[C@@H](F)C4)nc3)nn21 ; Cl] | 1. 78% 2. ¹H NMR (80 MHz, DMSO-d6) δ 9.06 (d, 1H), 8.62-8.22 (m, 4H), 7.66 (dd, ), 7.20-6.92 (m, 2H), 5.97-5.08 (m, 3H), 3.77 (s, 4H), 2.24-1.92 (m, 2H). 3. 365.12 |
| | 2 | |
| | (R)-2-(6-(3-fluoropyrrolidin-1- | |
| | yl)pyridin-3-yl)-5-(pyridin-3-yl)- | |
| | 4H-imidazo[1,5-b]pyrazol-6(5H)- | |
| | one hydrochloride | |
| 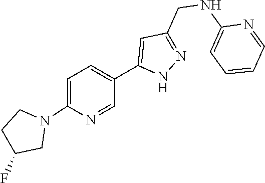 [SMILES: F[C@@H]1CCN(c2ccc(-c3cc(CNc4ccccn4)n[nH]3)cn2)C1] | 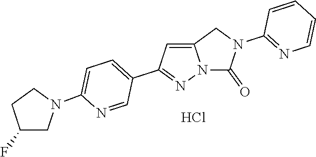 [SMILES: O=C1N(c2ccccn2)Cc2cc(-c3ccc(N4CC[C@@H](F)C4)nc3)nn21 ; Cl] | 1. 48% 2. ¹H NMR (80 MHz, DMSO-d6) δ 8.57 (d, 1H), 8.45 (dd, 1H), 8.37- 8.17 (m, 2H), 7.94 (td, 1H), 7.23 (t, 1H), 7.06-6.82 (m, 2H), 5.95- 5.00 (m, 3H), 4.13-3.70 (m, 4H), 2.32-1.99 (m, 2H). 3. 365.08 |
| | 3 | |
| | (R)-2-(6-(3-fluoropyrrolidin-1- | |
| | yl)pyridin-3-yl)-5-(pyridin-2-yl)- | |
| | 4H-imidazo[1,5-b]pyrazol-6(5H)- | |
| | one | |
| 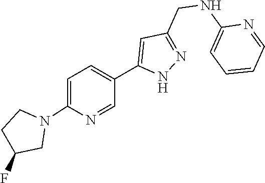 [SMILES: F[C@H]1CCN(c2ccc(-c3cc(CNc4ccccn4)n[nH]3)cn2)C1] | 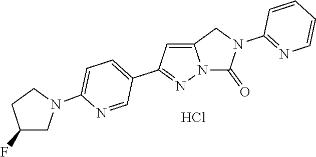 [SMILES: O=C1N(c2ccccn2)Cc2cc(-c3ccc(N4CC[C@H](F)C4)nc3)nn21 ; Cl] | 1. 62% 2. ¹H NMR (80 MHz, DMSO-d6) δ 8.58 (d, 1H), 8.46 (d, 1H), 8.33 (d, 1H), 8.23 (d, 1H), 7.95 (td, 1H), 7.23 (t, 1H), 7.05-6.85 (m, 2H), 5.96-5.05 (m, 3H), 4.07-3.50 (m, 4H), 2.30-1.84 (m, 2H). 3. 365.09 |
| | 4 | |
| | (S)-2-(6-(3-fluoropyrrolidin-1- | |
| | yl)pyridin-3-yl)-5-(pyridin-2-yl)- | |
| | 4H-imidazo[1,5-b]pyrazol-6(5H)- | |
| | one hydrochloride | |
| 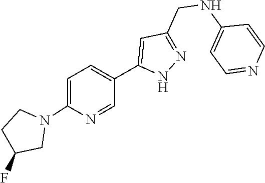 [SMILES: F[C@H]1CCN(c2ccc(-c3cc(CNc4ccncc4)n[nH]3)cn2)C1] | 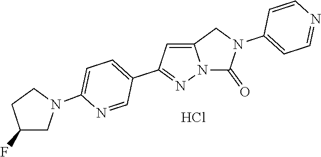 [SMILES: O=C1N(c2ccncc2)Cc2cc(-c3ccc(N4CC[C@H](F)C4)nc3)nn21 ; Cl] | 1. 73% 2. ¹H NMR (80 MHz, DMSO-d6) δ 8.85 (d, 2H), 8.65 (d, 1H), 8.36 8.08 (m, 3H), 7.11 (s, 1H), 6.84 (d, 1H), 5.97-5.09 (m, 3H), 4.11- 3.51 (m, 4H), 2.23-1.81 (m, 2H). 3. 365.12 |
| | 5 | |
| | (S)-2-(6-(3-fluoropyrrolidin-1- | |
| | yl)pyridin-3-yl)-5-(pyridin-4-yl)- | |
| | 4H-imidazo[1,5-b]pyrazol-6(5H)- | |
| | one hydrochloride | |
| 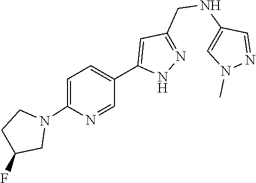 [SMILES: Cn1cc(NCc2cc(-c3ccc(N4CC[C@H](F)C4)nc3)[nH]n2)cn1] | 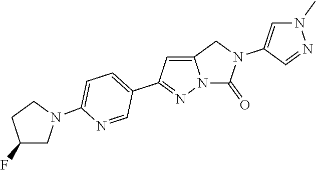 [SMILES: Cn1cc(N2Cc3cc(-c4ccc(N5CC[C@H](F)C5)nc4)nn3C2=O)cn1] | Only Step A was done 1. 35% 2. ¹H NMR (80 MHz, DMSO-d6) δ 8.65 (d, 1H), 8.21-7.91 (m, 2H), 7.66 (s, 1H), 6.88 (s, 1H), 6.60 (d, 1H), 5.45 (d, 1H), 4.93 (s, 2H), 3.99-3.47 (m, 7H), 2.21-1.86 (m, 2H). 3. 368.08 |
| | 6 | |
| | (S)-2-(6-(3-fluoropyrrolidin-1- | |
| | yl)pyridin-3-yl)-5-(1-methyl-1H- | |
| | pyrazol-4-yl)-4H-imidazo[1,5- | |
| | b]pyrazol-6(5H)-one | |
| 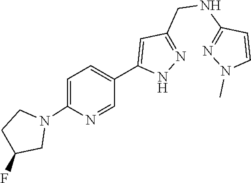 [SMILES: Cn1ccc(NCc2cc(-c3ccc(N4CC[C@H](F)C4)nc3)[nH]n2)n1] | 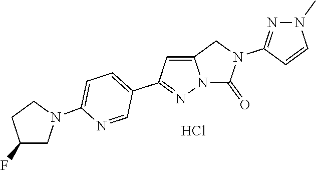 [SMILES: Cn1ccc(N2Cc3cc(-c4ccc(N5CC[C@H](F)C5)nc4)nn3C2=O)n1 ; Cl] | 1. 38% 2. ¹H NMR (80 MHz, DMSO-d6) δ 8.53 (d, 1H), 8.32 (dd, 1H), 7.74 (d, 1H), 7.02 (t, 2H), 6.55 (d, 1H), 5.54 (d, 1H), 5.01 (s, 2H), 4.23-3.63 (m, 7H), 2.28-1.79 (m, 2H). 3. 368.08 |
| | 7 | |
| | (S)-2-(6-(3-fluoropyrrolidin-1- | |
| | yl)pyridin-3-yl)-5-(1-methyl-1H- | |
| | pyrazol-3-yl)-4H-imidazo[1,5- | |
| | b]pyrazol-6(5H)-one | |
| | hydrochloride | |
| 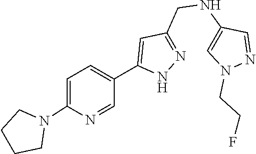 [SMILES: FCCn1cc(NCc2cc(-c3ccc(N4CCCC4)nc3)[nH]n2)cn1] | 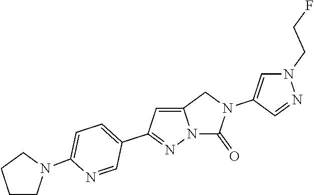 [SMILES: O=C1N(c2cnn(CCF)c2)Cc2cc(-c3ccc(N4CCCC4)nc3)nn21] | Only Step A was done 1. 45% 2. ¹H NMR (80 MHz, DMSO-d6) δ 8.63 (d, 1H), 8.19-7.90 (m, 2H), 7.74 (s, 1H), 6.87 (s, 1H), 6.54 (d, 1H), 5.01 (s, 2H), 4.79-4.17 (m, 4H), 3.57-3.38 (m, 4H), 2.10- 1.81 (m, 4H). 3. 382.13 |
| | 8 | |
| | 5-(1-(2-fluoroethyl)-1H-pyrazol- | |
| | 4-yl)-2-(6-(pyrrolidin-1-yl)pyridin- | |
| | 3-yl)-4H-imidazo[1,5-b]pyrazol- | |
| | 6(5H)-one | |
| 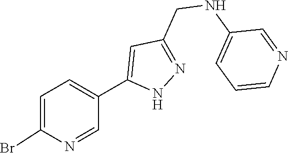 [SMILES: Brc1ccc(-c2cc(CNc3cccnc3)n[nH]2)cn1] | 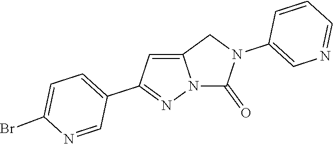 [SMILES: O=C1N(c2cccnc2)Cc2cc(-c3ccc(Br)nc3)nn21] | Only Step A was done 1. 75% 2. ¹H NMR (80 MHz, DMSO-d6) δ 8.98 (d, 2H), 8.44 (d, 1H), 8.38- 8.08 (m, 2H), 7.79 (d, 1H), 7.52 (dd, 1H), 7.17 (s, 1H), 5.20 (s, 2H). 3. 357.93 |
| | 9 | |
| | 2-(6-bromopyridin-3-yl)-5- | |
| | (pyridin-3-yl)-4H-imidazo[1,5- | |
| | b]pyrazol-6(5H)-one | |

### Example 10

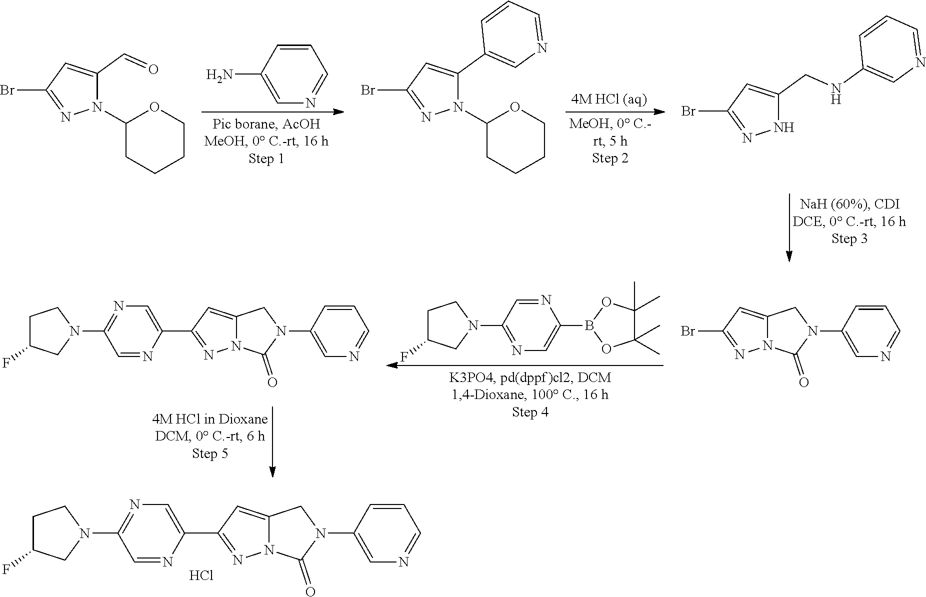

Step 1: To a solution of 3-bromo-1-(tetrahydro-2H-pyran-2-yl)-1H-pyrazole-5-carbaldehyde (6.0 g, 23.2 mmol) and pyridin-3-amine (2.1 g, 23.2 mmol) in methanol (240 mL) was added glacial AcOH (0.13 mL, 2.3 mmol) at RT under N₂. Then, the mixture was stirred for 30 min. After that Pic borane (2.4 g, 23.1 mmol) was added and the mixture was allowed stirred for another 16 h. The Progression of the reaction was monitored by TLC. The reaction mixture was quenched with sat. aq. NaHCO₃ solution and the product was extracted with DCM three times (100 mL×3). The extract was dried over Na₂SO₄ and concentrated under vacuum. The obtained crude mass was purified by column chromatography over silica gel (230-400 mesh) eluted in 2% MeOH in DCM to afford N-((3-bromo-1-(tetrahydro-2H-pyran-2-yl)-1H-pyrazol-5-yl) methyl) pyridine-3-amine as brownish liquid (4.2 g, 53%).

¹H NMR (DMSO-d6) δ 8.00 (d, 1H), 7.80 (dd, 1H), 7.08 (dd, 1H), 6.95 (dq, 1H), 6.39 (t, 1H), 6.30 (s, 1H), 5.51 (dd, 1H), 4.40 (m, 2H), 3.87 (m, 1H), 3.66 (m, 1H), 2.18 (m, 1H), 1.97 (m, 1H), 1.88 (td, 1H), 1.64 (m, 1H), 1.51 (m, 2H).

MS (ESI): 338.38 [M+H]+

Step 2: To a stirred solution of N-((3-bromo-1-(tetrahydro-2H-pyran-2-yl)-1H-pyrazol-5-yl) methyl) pyridine-3-amine (4.2 g, 12.5 mmol) in MeOH (100 mL) was added aq. 4M HCl (29.5 mL, 7.0 vol) at 0° C. under N₂ atmosphere and stirred at RT for 5 h. The reaction time was monitored by TLC. After completion, the reaction mixture was cooled to 0° C. and quenched with saturated aq. NaHCO₃ until the resultant mixture pH reaches up to 8-9. The solvent was removed under vacuum and the product was extracted with DCM three times (80 mL X×3). The combined organic layer was dried over Na₂SO₄ and concentrated under vacuum. The obtained mass was washed with hexane three times (15 mL×3), dried under vacuum to afford N-((3-bromo-1H-pyrazol-5-yl) methyl) pyridin-3-amine as yellow solid (400 mg, 80%), directly used for next step without any further purification.

¹H NMR (DMSO-d6) δ 13.10 (s, 1H), 7.99 (d, 1H), 7.80 (dd, 1H), 7.08 (dd, 1H), 6.93 (dq, 1H), 6.32 (t, 1H), 6.27 (s, 1H), 4.28 (d, 2H).

MS (ESI): 254.83 [M+H]+

Step 3: To an ice cool solution of N-((3-bromo-1H-pyrazol-5-yl) methyl) pyridin-3-amine (2.5 g, 9.8 mmol) in 1, 2-DCE (250 mL) was added NaH (60% dispersed in mineral oil) (120 mg, 4.9 mmol) under N₂ atmosphere. Then, the mixture was allowed to RT and kept for 30 min. Then, CDI (16.0 g, 99 mmol) was added to the reaction mixture and stirred at RT for 16 h. After completion, the reaction mixture was quenched with ice cold water and the product was extracted with DCM three times (70 mL×3). The extract was dried over Na₂SO₄ and concentrated under vacuum. The residue was purified by silica gel chromatography (230-400 mesh) eluted in 3% MeOH in DCM to yield 2-bromo-5-(pyridin-3-yl)-4,5-dihydro-6H-imidazo[1,5-b]pyrazol-6-one as yellow solid (1.65 g, 61%).

¹H NMR (DMSO-d6) δ 8.94 (m, 1H), 8.44 (dd, 1H), 8.16 (dq, 1H), 7.52 (dd, 1H), 6.74 (t, 1H), 5.14 (d, 2H).

MS (ESI): 279.04 [M]+

Step 4: In an oven-dried screw capped vial was added 2-bromo-5-(pyridin-3-yl)-4,5-dihydro-6H-imidazo[1,5-b]pyrazol-6-one (120 mg, 0.43 mmol), boronic ester (600 mg, 0.6 mmol), K3PO4 (166 mg, 1.3 mmol) and 1,4-dioxane (5.0 mL) under an argon atmosphere. The reaction mixture was degassed with argon for 15 min. Then, Pd(dppf)Cl₂·DCM (35 mg, 0.043 mmol) was added and the mixture was heated to 100° C. for 16 h. The reactants were consumed as monitored by TLC. After that the reaction mixture was quenched with ice-water and extracted with DCM three times (10 mL×3). The organic layer was dried over Na₂SO₄, concentrated and purified by silica gel chromatography (230-400 mesh) eluted in 3% MeOH in DCM to get (R)-2-(5-(3-fluoropyrrolidin-1-yl)pyrazin-2-yl)-5-(pyridin-3-yl)-4,5-dihydro-6H-imidazo[1,5-b]pyrazol-6-one as white solid (15 mg, 10%)

¹H NMR (DMSO-d6) δ 8.97 (d, 1H), 8.75 (d, 1H), 8.43 (q, 1H), 8.21 (m, 1H), 8.10 (d, 1H), 7.52 (q, 1H), 6.90 (s, 1H), 5.51 (d, 1H), 5.17 (d, 2H), 3.78 (m, 3H), 3.54 (m, 1H), 2.25 (m, 2H).

Step 5: To a stirred solution of (R)-2-(5-(3-fluoropyrrolidin-1-yl)pyrazin-2-yl)-5-(pyridin-3-yl)-4,5-dihydro-6H-imidazo[1,5-b]pyrazol-6-one (15 mg, 0.041 mmol) in DCM (2.0 mL) was added 4M HCl in 1,4-Dioxane (0.075 mL) at 0° C. under N2 atmosphere and stirred at RT for 6 h. After completion of the reaction, solvent was evaporated, washed with pentane, dried under vacuum to afford as white solid (10 mg, 62%).

¹H NMR (500 MHz, DMSO-D6) δ 9.08 (d, 1H), 8.75 (d, 1H), 8.53 (dt, 1H), 8.39 (d, 1H), 8.10 (d, 1H), 7.79-7.68 (m, 1H), 7.03-6.82 (m, 1H), 5.51 (d, 1H), 5.19 (s, 2H), 3.84-3.64 (m, 3H), 3.64-3.52 (m, 1H), 2.41-2.13 (m, 2H).

LCMS: 365.95 [M]+

### Example 11

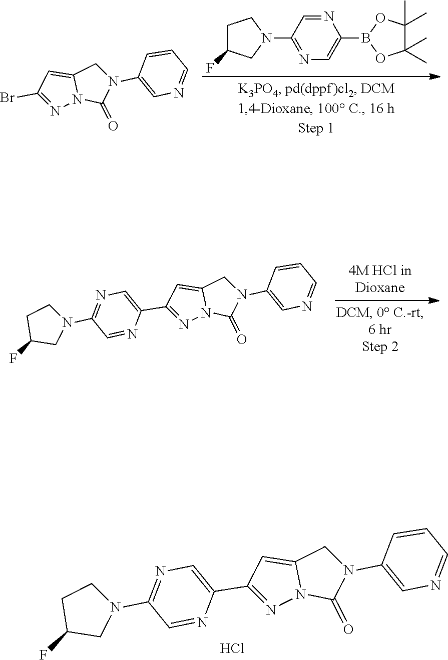

Step 1: In an oven-dried screw capped vial was added 2-bromo-5-(pyridin-3-yl)-4,5-dihydro-6H-imidazo[1,5-b]pyrazol-6-one (120 mg, 0.43 mmol), boronic ester (600 mg, 0.6 mmol), K₃PO₄ (166 mg, 1.3 mmol) and 1,4-dioxane (5.0 mL) under an argon atmosphere. The reaction mixture was degassed with argon for 15 min. Then Pd(dppf)Cl₂·DCM (35 mg, 0.043 mmol) was added and the mixture was heated to 100° C. for 16 h. The reactants were consumed as monitored by TLC. After that the reaction mixture was quenched with ice-water and extracted with DCM three times (10 mL×3). The organic layer was dried over Na₂SO₄, concentrated and purified by silica gel chromatography (230-400 mesh) eluted in 3% MeOH in DCM to get (S)-2-(5-(3-fluoropyrrolidin-1-yl)pyrazin-2-yl)-5-(pyridin-3-yl)-4,5-dihydro-6H-imidazo[1,5-b]pyrazol-6-one as white solid (20 mg, 13%).

¹H NMR (500 MHz, DMSO-D6) δ 8.97 (d, 1H), 8.75 (d, 1H), 8.43 (dd, 1H), 8.20 (ddd, 1H), 8.10 (d, 1H), 7.52 (dd, 1H), 6.96-6.86 (m, 1H), 5.51 (d, 1H), 5.17 (s, 2H), 3.93-3.62 (m, 3H), 3.62-3.46 (m, 1H), 2.35-2.10 (m, 2H).

LCMS: 365.8 [M]+

Step 2: To a stirred solution of (S)-2-(5-(3-fluoropyrrolidin-1-yl)pyrazin-2-yl)-5-(pyridin-3-yl)-4,5-dihydro-6H-imidazo[1,5-b]pyrazol-6-one (20 mg, 0.054 mmol) in DCM (1.6 mL, 80 vol) was added 4M HCl in 1,4-Dioxane (0.1 mL, 5.0 vol) at 0° C. under N₂ atmosphere and stirred at RT for 5 h. Then, solvent was evaporated, washed with pentane, dried under vacuum to afford (S)-2-(5-(3-fluoropyrrolidin-1-yl)pyrazin-2-yl)-5-(pyridin-3-yl)-4,5-dihydro-6H-imidazo[1,5-b]pyrazol-6-one hydrogen chloride salt as white solid (15 mg, 71%).

¹H NMR (500 MHz, DMSO-D6) δ 9.21-9.16 (m, 1H), 8.76 (d, 1H), 8.68-8.55 (m, 2H), 8.11 (d, 1H), 7.92 (dd, 1H), 6.94 (s, 1H), 5.51 (d, 1H), 5.21 (s, 2H), 3.94-3.64 (m, 3H), 3.64-3.52 (m, 1H), 2.42-2.11 (m, 2H).

LCMS: 365.9 [M]+

### Example 12

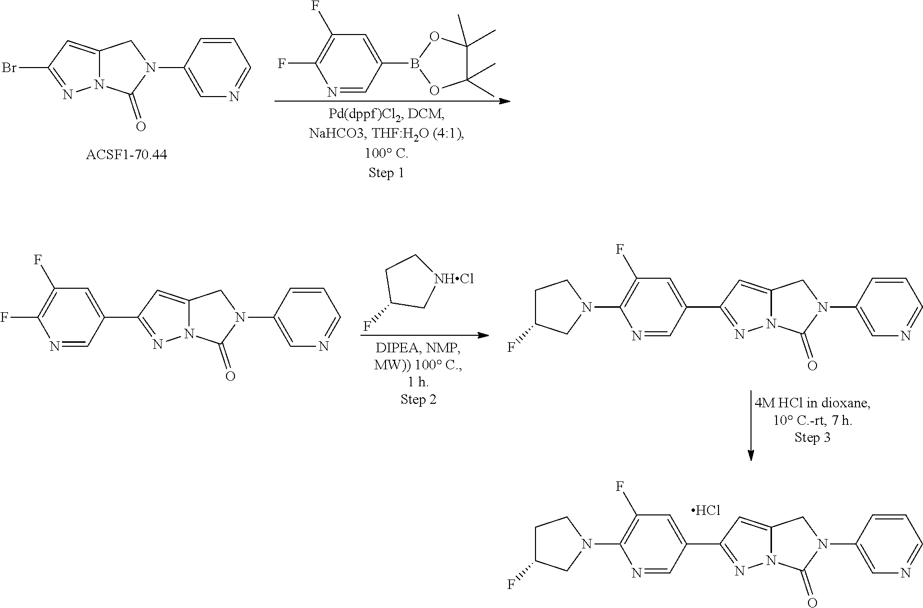

Step 1: In an oven-dried screw capped vial was added 2-bromo-5-(pyridin-3-yl)-4,5-dihydro-6H-imidazo[1,5-b]pyrazol-6-one (250 mg, 0.89 mmol), boronic ester (323 mg, 1.39 mmol), NaHCO₃ (376 mg, 4.48 mmol) and (THF/H₂O) (4:1, 5.0 mL, 20 vol) under argon atmosphere. The reaction mixture was degassed with argon for 15 min. Then, Pd(dppf)Cl₂·DCM (73 mg, 0.089 mmol) was added and the mixture was heated to 100° C. for 5 h. After that the reaction mixture was quenched with ice-water and extracted with EtOAc three times (30 mL X×3). The organic layer was dried over Na₂SO₄, concentrated and purified by silica gel chromatography (230-400 mesh) eluted in 3% MeOH in DCM to get 2-(5,6-difluoropyridin-3-yl)-5-(pyridin-3-yl)-4,5-dihydro-6H-imidazo[1,5-b]pyrazol-6-one as brown solid (100 mg, 35%).

¹H NMR (DMSO-d6) δ 8.98 (d, 1H), 8.67 (t, 1H), 8.57 (m, 1H), 8.45 (dd, 1H), 8.21 (dq, 1H), 7.53 (dd, 1H), 7.18 (s, 1H), 5.21 (s, 2H).

MS (ESI): 314.56 [M+H]+

Step 2: 2-(5,6-difluoropyridin-3-yl)-5-(pyridin-3-yl)-4,5-dihydro-6H-imidazo[1,5-b]pyrazol-6-one (50 mg, 0.15 mmol), (R)-3-fluoropyrrolidine hydrogen chloride (20 mg, 0.23 mmol), DIPEA (0.06 mL, 0.48 mmol), and NMP (2.0 mL) was taken in an oven-dried micro wave vial under argon atmosphere. The reaction mixture was heated under microwave irradiation at 100° C. for 1 h. After completion, the reaction mixture was quenched with ice cold water (5 mL). The crude reaction mass was filtered through Buchner funnel and the obtained mass was washed with hexane three times (3 mL×3), dried under high vacuum to afford (R)-2-(5-fluoro-6-(3-fluoropyrrolidin-1-yl)pyridin-3-yl)-5-(pyridin-3-yl)-4,5-dihydro-6H-imidazo[1,5-b]pyrazol-6-one as off white solid (32 mg, 52%).

¹H NMR (500 MHz, DMSO-D6) δ 8.97 (d, 1H), 8.54 (s, 1H), 8.42 (d, 1H), 8.27-8.13 (m, 1H), 7.92 (d, 1H), 7.51 (dd, 1H), 7.00 (s, 1H), 5.44 (d, 1H), 5.16 (s, 2H), 3.85 (td, 3H), 3.69 (q, 1H), 2.31-2.03 (m, 2H).

LCMS: 382.9 [M]+

Step 3: To a stirred solution of (R)-2-(5-fluoro-6-(3-fluoropyrrolidin-1-yl)pyridin-3-yl)-5-(pyridin-3-yl)-4,5-dihydro-6H-imidazo[1,5-b]pyrazol-6-one (32 mg, 0.08 mmol) in 1,4-dioxane (1.0 mL, 30 vol) was added 4M HCl in 1,4-Dioxane (0.16 mL, 5.0 vol.) at 0° C. under N₂ atmosphere and stirred at RT for 7 h. Then, the solvent was evaporated, washed with pentane, dried under vacuum to afford (R)-2-(5-fluoro-6-(3-fluoropyrrolidin-1-yl)pyridin-3-yl)-5-(pyridin-3-yl)-4,5-dihydro-6H-imidazo[1,5-b]pyrazol-6-one hydrogen chloride salt as off white solid (30 mg, 85%).

¹H NMR (500 MHz, DMSO-D6) δ 9.09 (d, 1H), 8.54 (s, 2H), 8.42 (d, 1H), 7.95 (dd, 1H), 7.74 (dd, 1H), 7.03 (s, 1H), 5.44 (d, 1H), 5.19 (d, 2H), 3.92-3.65 (m, 4H), 2.32-2.03 (m, 2H).

LCMS: 383.2 [M+H]+

### Example 13

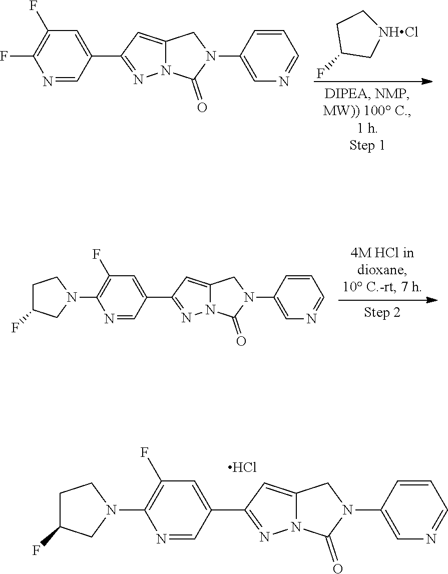

Step 1:2-(5,6-difluoropyridin-3-yl)-5-(pyridin-3-yl)-4,5-dihydro-6H-imidazo[1,5-b]pyrazol-6-one (30 mg, 0.095 mmol), (S)-3-fluoropyrrolidine hydrogen chloride (17 mg, 0.14 mmol), DIPEA (0.05 mL, 0.18 mmol), and NMP (0.6 mL, 20 vol.) was taken in an oven-dried micro wave vial under argon atmosphere. The reaction mixture was heated under microwave irradiation at 100° C. for 1 h. After completion, the reaction mixture was quenched with ice cold water (5 mL). The crude reaction mass was filtered through Büchner funnel and the obtained mass was washed with hexane three times (3 mL×3), dried under high vacuum to afford (S)-2-(5-fluoro-6-(3-fluoropyrrolidin-1-yl) pyridin-3-yl)-5-(pyridin-3-yl)-4,5-dihydro-6H-imidazo[1,5-b]pyrazol-6-one as off white solid (20 mg, 55%).

¹H NMR (400 MHz, DMSO-D₆) δ 8.96 (d, 1H), 8.54 (t, 1H), 8.42 (dd, 1H), 8.20 (d, 1H), 7.94 (dd, 1H), 7.51 (dd, 1H), 7.00 (s, 1H), 5.44 (d, 1H), 5.16 (s, 2H), 3.98-3.62 (m, 4H), 2.33-1.98 (m, 2H).

LCMS: 382.9 [M]+;

Step 2: To a stirred solution of (S)-2-(5-fluoro-6-(3-fluoropyrrolidin-1-yl) pyridin-3-yl)-5-(pyridin-3-yl)-4,5-dihydro-6H-imidazo[1,5-b]pyrazol-6-one (20 mg, 0.05 mmol) in 1,4-dioxane (0.6 mL, 30 vol.) was added 4M HCl in 1,4-Dioxane (0.1 mL, 5.0 vol.) at 0° C. under N2 atmosphere and stirred at RT for 7 h. After completion of the reaction, solvent was evaporated, washed with pentane, dried under vacuum to afford (S)-2-(5-fluoro-6-(3-fluoropyrrolidin-1-yl) pyridin-3-yl)-5-(pyridin-3-yl)-4,5-dihydro-6H-imidazo[1,5-b]pyrazol-6-one hydrogen chloride salt as yellow solid (15 mg, 71%).

¹H NMR (500 MHz, DMSO-D6) δ 9.11 (d, 1H), 8.60-8.50 (m, 2H), 8.45 (d, 1H), 7.95 (dd, 1H), 7.78 (dd, 1H), 7.03 (s, 1H), 5.44 (d, 1H), 5.19 (s, 2H), 3.96-3.75 (m, 3H), 3.75-3.62 (m, 1H), 2.32-2.01 (m, 2H).

LCMS: 383.3 [M+H]+;

### Example 14

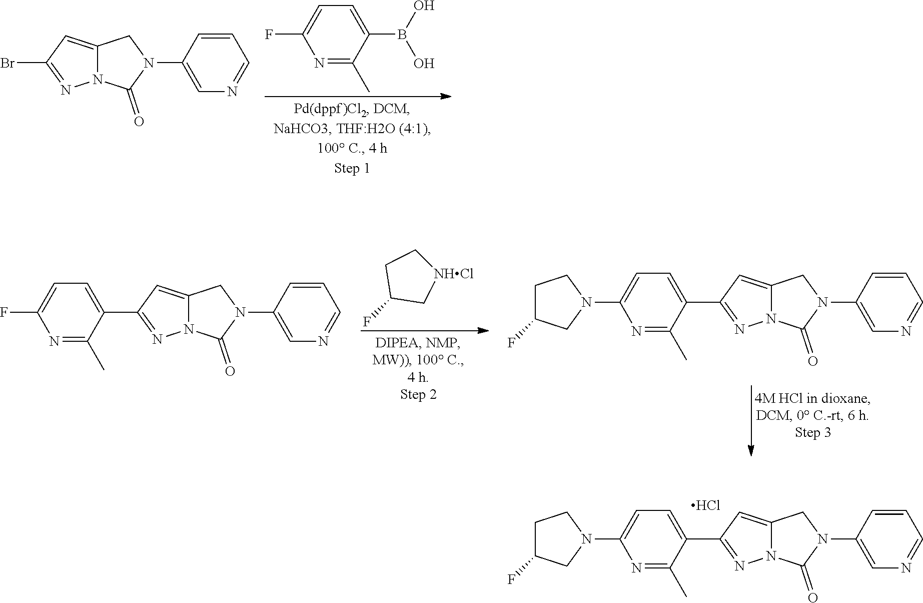

Step 1: In an oven-dried screw capped vial was added 2-bromo-5-(pyridin-3-yl)-4,5-dihydro-6H-imidazo[1,5-b]pyrazol-6-one (450 mg, 1.6 mmol), boronic acid (500 mg, 3.2 mmol), NaHCO₃ (675 mg, 8.0 mmol) and THF:H2O (4:1, 9.0 mL, 20 vol) under an argon atmosphere. The reaction mixture was degassed with argon for 15 min. Then Pd(dppf)Cl₂·DCM (130 mg, 0.16 mmol) was added and the mixture was heated to 100° C. for 4 h. After that the reaction mixture was quenched with ice-water and extracted with DCM three times (30 mL x). The organic layer was dried over Na₂SO₄, concentrated and purified by silica gel chromatography (100-200 mesh) eluted in 2% MeOH in DCM to get 2-(6-fluoro-2-methylpyridin-3-yl)-5-(pyridin-3-yl)-4,5-dihydro-6H-imidazo [1,5-b]pyrazol-6-one as yellow solid (300 mg, 60%).

¹H NMR (400 MHz, DMSO-D6) δ 8.97 (d, 1H), 8.42 (d, 1H), 8.20 (d, 1H), 7.81 (d, 1H), 7.52 (dd, 1H), 6.77 (s, 1H), 6.45 (d, 1H), 5.45 (d, 1H), 5.16 (s, 2H), 3.89-3.38 (m, 4H), 2.60 (s, 3H), 2.36-2.04 (m, 2H).

LCMS: 310.9 [M+H]+

Step 2: 2-(6-fluoro-2-methylpyridin-3-yl)-5-(pyridin-3-yl)-4,5-dihydro-6H-imidazo [1,5-b]pyrazol-6-one (80 mg, 0.16 mmol), (R)-3-fluoropyrrolidine hydrogen chloride (82 mg, 0.32 mmol), DIPEA (0.16 mL, 0.48 mmol), and NMP (2 mL, 20 vol) was taken in an oven-dried micro wave vial under argon atmosphere. The reaction mixture was heated under microwave irradiation at 160° C. for 4 h. After completion, the reaction mixture was quenched with ice cold water (10 mL). The crude reaction mass was filtered through Buchner funnel and the obtained mass was washed with hexane three times (5 mL×3), dried under high vacuum to afford (R)-2-(6-(3-fluoropyrrolidin-1-yl)-2-methylpyridin-3-yl)-5-(pyridin-3-yl)-4,5-dihydro-6H-imidazo[1,5-b]pyrazol-6-one as off-white solid (70 mg, 71%).

¹H NMR (DMSO-d6) δ 8.98 (d, 1H), 8.43 (d, 1H), 8.20 (d, 1H), 7.81 (d, 1H), 7.52 (dd, 1H), 6.77 (s, 1H), 6.46 (d, 1H), 5.46 (d, 1H), 5.16 (s, 2H), 3.70 (m, 3H), 3.46 (m, 1H), 2.60 (s, 3H), 2.20 (m, 2H).

LCMS: 379.4 [M+H]+

Step 3: To a stirred solution of (R)-2-(6-(3-fluoropyrrolidin-1-yl)-2-methylpyridin-3-yl)-5-(pyridin-3-yl)-4,5-dihydro-6H-imidazo[1,5-b]pyrazol-6-one (70 mg, 0.18 mmol) in DCM (7 mL, 100 vol) was added 4M HCl in 1,4-dioxane (0.7 mL, 10 vol) at 0° C. under N₂ atmosphere and stirred at RT for 6 h. After completion of the reaction, solvent was evaporated, washed with pentane, dried under vacuum to afford (R)-2-(6-(3-fluoropyrrolidin-1-yl)-2-methylpyridin-3-yl)-5-(pyridin-3-yl)-4,5-dihydro-6H-imidazo[1,5-b]pyrazol-6-one hydrogen chloride salt as a white solid (60 mg, 78%).

¹H NMR (400 MHz, DMSO-D6) δ 9.12 (d, 1H), 8.58 (dd, 1H), 8.52-8.42 (m, 1H), 8.21 (d, 1H), 7.80 (dd, 1H), 7.03 (d, 1H), 6.96 (d, 1H), 5.69-5.43 (m, 1H), 5.24 (s, 2H), 4.11-3.64 (m, 4H), 2.83 (s, 3H), 2.44-2.12 (m, 2H).

LCMS: 379.4 [M+H]+

### Example 15

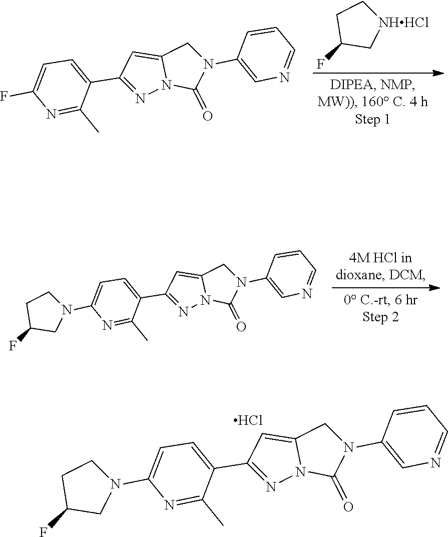

Step 1: 2-(6-fluoro-2-methylpyridin-3-yl)-5-(pyridin-3-yl)-4,5-dihydro-6H-imidazo [1,5-b]pyrazol-6-one (80 mg, 0.26 mmol), (S)-3-fluoropyrrolidine hydrogen chloride (66 mg, 0.52 mmol), DIPEA (0.13 mL, 0.52 mmol), and NMP (1.6 mL, 20 vol) was taken in an oven-dried micro wave vial under argon atmosphere. The reaction mixture was heated under microwave irradiation at 160° C. for 4 h. After completion, the reaction mixture was quenched with ice cold water (10 mL). The crude reaction mass was filtered through Buchner funnel and the obtained mass was washed with hexane three times (5 mL×3), dried under high vacuum to afford (S)-2-(6-(3-fluoropyrrolidin-1-yl)-2-methylpyridin-3-yl)-5-(pyridin-3-yl)-4,5-dihydro-6H-imidazo[1,5-b]pyrazol-6-one as off-white solid (80 mg, 81%).

¹H NMR (500 MHz, DMSO-D6) δ 8.97 (d, 1H), 8.43 (dd, 1H), 8.20 (d, 1H), 7.81 (d, 1H), 7.52 (dd, 1H), 6.77 (s, 1H), 6.46 (d, 1H), 5.46 (d, 1H), 5.16 (s, 2H), 3.88-3.55 (m, 3H), 3.55-3.41 (m, 1H), 2.60 (s, 3H), 2.33-2.14 (m, 2H).

LCMS: 378.90 [M]+

Step 2: To a stirred solution of (S)-2-(6-(3-fluoropyrrolidin-1-yl)-2-methylpyridin-3-yl)-5-(pyridin-3-yl)-4,5-dihydro-6H-imidazo[1,5-b]pyrazol-6-one (80 mg, 0.21 mmol) in DCM (8 mL) was added 4M HCl in 1,4-Dioxane (0.8 mL) at 0° C. under N₂ atmosphere and stirred at RT for 6 h. After completion of the reaction, solvent was evaporated, washed with pentane, dried under vacuum to afford (S)-2-(6-(3-fluoropyrrolidin-1-yl)-2-methylpyridin-3-yl)-5-(pyridin-3-yl)-4,5-dihydro-6H-imidazo[1,5-b]pyrazol-6-onehydrogen chloride salt as white solid (80 mg, 91%).

¹H NMR (400 MHz, DMSO-D6) δ 9.15 (d, 1H), 8.61 (dd, 1H), 8.59-8.48 (m, 1H), 8.23 (d, 1H), 7.87 (dd, 1H), 7.05 (d, 1H), 6.97 (s, 1H), 5.58 (d, 1H), 5.25 (s, 2H), 4.15-3.79 (m, 3H), 3.79-3.63 (m, 1H), 2.85 (s, 3H), 2.48-2.11 (m, 2H).

LCMS: 378.85 [M]+;

### Example 16

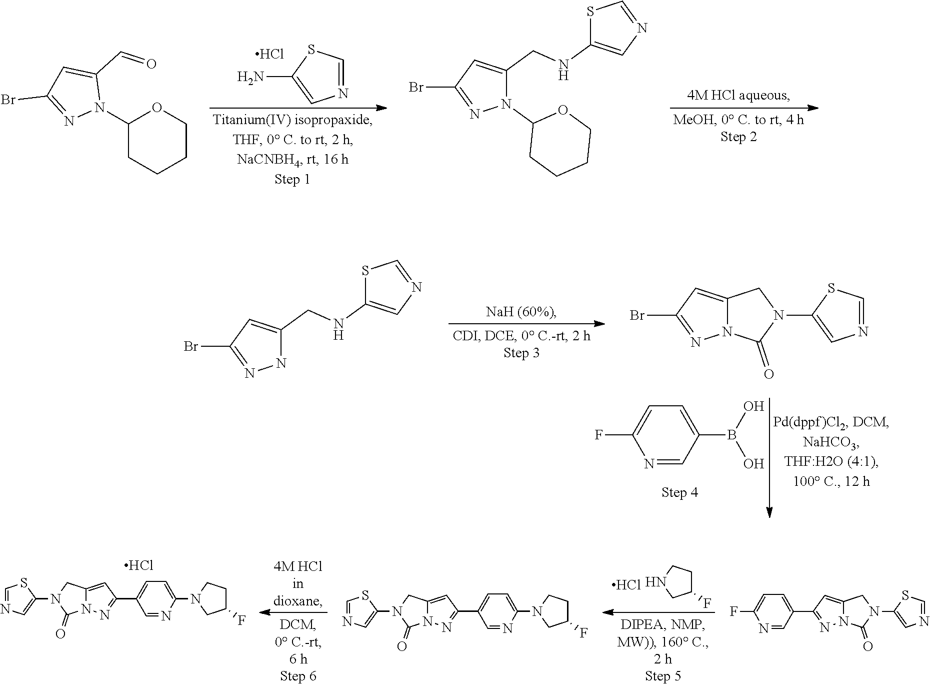

Step 1: To a stirred solution of 3-bromo-1-(tetrahydro-2H-pyran-2-yl)-1H-pyrazole-5-carbaldehyde (2.0 g, 7.7 mmol) and thiazol-5-amine hydrogen chloride salt (3.1 g, 23.1 mmol) in THF (120 mL) was added titanium (IV) isopropaxide (6.8 mL, 23.1 mmol) under N₂, and kept for 2 h. Then, sodium cyano borohydride (0.72 g, 11.5 mmol) was added and the mixture was stirred at RT for 16 h. After completion of the reaction, solvent was removed under high vacuum. The reaction mixture was quenched with sat. aq. NaHCO₃ (40 mL) and EtOAc (80 mL) was added with stirring. The resulting inorganic precipitate was filtered through celite bed. Collected the organic layer from filtrate and aqueous layer was extracted with EtOAc three times (60 mL×3). Combined organic layers were dried over Na₂SO₄ and concentrated under vacuum. The obtained crude mass was purified by column chromatography over silica gel (100-200 mesh) eluted in 3% MeOH in DCM to afford N-((3-bromo-1-(tetrahydro-2H-pyran-2-yl)-1H-pyrazol-5-yl)methyl) thiazol-5-amine as brown solid (1.5 g, 57%).

Step 2: To a stirred solution of N-((3-bromo-1-(tetrahydro-2H-pyran-2-yl)-1H-pyrazol-5-yl)methyl) thiazol-5-amine (1.5 g, 4.3 mmol) in MeOH (36 mL, 24 vol) was added aq. 4M HCl (15 mL, 10 vol) at 0° C. under N₂ atmosphere and stirred at RT for 4 h. After completion of the reaction, the reaction mixture was cooled to 0° C. and quenched with saturated aq. NaHCO₃ until the resultant mixture pH reaches up to 8-9. The solvent was removed under vacuum and the product was extracted with DCM three times (50 mL×3). The combined organic layer was dried over Na₂SO₄ and concentrated under vacuum. The obtained mass was washed with hexane three times (8 mL×3), dried under vacuum to afford N-((3-bromo-1H-pyrazol-5-yl) methyl) thiazol-5-amine as yellow solid (1.0 g, 90%) directly used for next step without any further purification. MS (ESI): 258.99 [M+H]+

Step 3: To an ice cool solution of N-((3-bromo-1H-pyrazol-5-yl) methyl) thiazol-5-amine (1.0 g, 3.8 mmol) in 1, 2-DCE (15 mL) was added NaH (60% dispersed in mineral oil) (92 mg, 1.9 mmol) under N₂ atmosphere. Then, the mixture was allowed to RT and kept for 30 min. Then, CDI (6.2 g, 38.6 mmol) was added to the reaction mixture and stirred at RT for 2 h. The reaction mixture was quenched with ice cold water and the product was extracted with DCM three times (40 mL×3). The extract was dried over Na₂SO₄ and concentrated under vacuum. The residue was purified by silica gel chromatography (100-200 mesh) eluted in 2% MeOH in DCM to yield 2-bromo-5-(thiazol-5-yl)-4,5-dihydro-6H-imidazo[1,5-b]pyrazol-6-one as white solid (800 mg, 72%).

¹H NMR (DMSO-d6) δ 8.82 (d, 1H), 7.79 (d, 1H), 6.74 (s, 1H), 5.10 (m, 2H).

MS (ESI): 284.94 [M+H]+

Step 4: In an oven-dried screw capped vial was added 2-bromo-5-(thiazol-5-yl)-4,5-dihydro-6H-imidazo[1,5-b]pyrazol-6-one (400 mg, 1.4 mmol), boronic acid (395 mg, 2.8 mmol), NaHCO₃ (590 mg, 7.0 mmol) and (THF: H₂O) (4:1, 8.0 mL) under an argon atmosphere. The reaction mixture was degassed with argon for 15 min. Then Pd(dppf)Cl₂·DCM (115 mg, 0.14 mmol) was added and the mixture was heated to 100° C. for 12 h. After that the reaction mixture was quenched with ice-water and extracted with DCM three times (20 mL×3). The organic layer was dried over Na₂SO₄, concentrated and purified by silica gel chromatography (100-200 mesh) eluted in 2% MeOH in DCM to get 2-(6-fluoropyridin-3-yl)-5-(thiazol-5-yl)-4,5-dihydro-6H-imidazo[1,5-b]pyrazol-6-one as white solid (130 mg, 30%).

¹H NMR (DMSO-d6) δ 8.84 (d, 1H), 8.82 (s, 1H), 8.53 (m, 1H), 7.81 (s, 1H), 7.34 (dd, 1H), 7.17 (s, 1H), 5.17 (s, 2H).

LCMS: 302.15 [M+H]+

Step 5: 2-(6-fluoropyridin-3-yl)-5-(thiazol-5-yl)-4,5-dihydro-6H-imidazo[1,5-b] pyrazol-6-one (50 mg, 0.16 mmol), (R)-3-fluoropyrrolidine hydrogen chloride (30 mg, 0.24 mmol), DIPEA (0.08 mL, 0.49 mmol), and NMP (0.5 mL, 10 vol) was taken in an oven-dried microwave vial under argon atmosphere. The reaction mixture was heated under microwave irradiation at 160° C. for 2 h. The reaction mixture was quenched with ice cold water (3 mL). The crude reaction mass was filtered through Büchner funnel. The obtained mass was washed with hexane three times (3 mL×3), dried under high vacuum to afford (R)-2-(6-(3-fluoropyrrolidin-1-yl)pyridin-3-yl)-5-(thiazol-5-yl)-4,5-dihydro-6H-imidazo[1,5-b]pyrazol-6-one as white solid (20 mg, 32%).

¹H NMR (500 MHz, DMSO-D6) δ 7.97 (d, 1H), 7.85 (d, 1H), 7.23 (dd, 1H), 6.95 (d, 1H), 6.14 (d, 1H), 5.80 (d, 1H), 4.65 (d, 1H), 4.30 (s, 2H), 3.03-2.74 (m, 3H), 2.74-2.59 (m, 1H), 1.52-1.27 (m, 2H).

LCMS: 393.15 [M+Na]+

Step 6: To a stirred solution of (R)-2-(6-(3-fluoropyrrolidin-1-yl)pyridin-3-yl)-5-(thiazol-5-yl)-4,5-dihydro-6H-imidazo[1,5-b]pyrazol-6-one (20 mg, 0.05 mmol) in DCM (0.2 mL, 10 vol) was added 4M HCl in 1,4-Dioxane (0.1 mL, 5.0 vol) at 0° C. under N₂ atmosphere and stirred at RT for 6 h. After completion of the reaction, solvent was evaporated, washed with pentane, dried under vacuum to afford (R)-2-(6-(3-fluoropyrrolidin-1-yl) pyridin-3-yl)-5-(thiazol-5-yl)-4,5-dihydro-6H-imidazo[1,5-b]pyrazol-6-one hydrogen chloride salt as white solid (18 mg, 85%).

¹H NMR (400 MHz, DMSO-D6) δ 8.81 (d, 1H), 8.54 (d, 1H), 8.47-8.29 (m, 1H), 7.80 (d, 1H), 7.22-6.95 (m, 2H), 5.55 (d, 1H), 5.15 (s, 2H), 4.01-3.77 (m, 4H), 2.35-2.04 (m, 2H).

LCMS: 371.15 [M+H]+

### Example 17

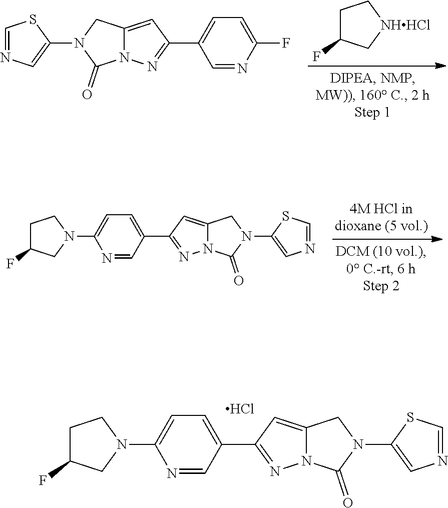

Step 1: 2-(6-fluoropyridin-3-yl)-5-(thiazol-5-yl)-4,5-dihydro-6H-imidazo[1,5-b] pyrazol-6-one (50 mg, 0.16 mmol), (S)-3-fluoropyrrolidine hydrogen chloride (30 mg, 0.24 mmol), DIPEA (0.08 mL, 0.49 mmol), and NMP (0.5 mL) was taken in an oven-dried microwave vial under argon atmosphere. The reaction mixture was heated under microwave irradiation at 160° C. for 2 h. After completion, the reaction mixture was quenched with ice cold water (3 mL). The crude reaction mass was filtered through Büchner funnel. The obtained mass was washed with hexane three times (3 mL×3), dried under high vacuum to afford (S)-2-(6-(3-fluoropyrrolidin-1-yl)pyridin-3-yl)-5-(thiazol-5-yl)-4,5-dihydro-6H-imidazo[1,5-b]pyrazol-6-one as white solid (20 mg, 32%).

¹H NMR (400 MHz, DMSO-D6) δ 8.79 (d, 1H), 8.68 (d, 1H), 8.05 (dd, 1H), 7.77 (d, 1H), 6.96 (s, 1H), 6.62 (d, 1H), 5.47 (d, 1H), 5.12 (s, 2H), 3.87-3.56 (m, 3H), 3.56-3.43 (m, 1H), 2.38-2.05 (m, 2H).

LCMS: 370.2 [M]+

Step 2: To a stirred solution of (S)-2-(6-(3-fluoropyrrolidin-1-yl)pyridin-3-yl)-5-(thiazol-5-yl)-4,5-dihydro-6H-imidazo[1,5-b]pyrazol-6-one (20 mg, 0.05 mmol) in DCM (0.2 mL, 10 vol) was added 4M HCl in 1,4-Dioxane (0.1 mL) at 0° C. under N₂ atmosphere and stirred at RT for 6 h. After completion of the reaction, solvent was evaporated, washed with pentane, dried under vacuum to afford (S)-2-(6-(3-fluoropyrrolidin-1-yl)pyridin-3-yl)-5-(thiazol-5-yl)-4,5-dihydro-6H-imidazo[1,5-b]pyrazol-6-one hydrogen chloride salt as white solid (18 mg, 85%).

¹H NMR (400 MHz, DMSO-D6) δ 8.81 (s, 1H), 8.53 (d, 1H), 8.38 (d, 1H), 7.80 (s, 1H), 7.11 (d, 2H), 5.56 (d, 1H), 5.16 (s, 2H), 4.00-3.81 (m, 4H), 2.36-2.07 (m, 2H).

LCMS: 370.9 [M]+

### Example 18

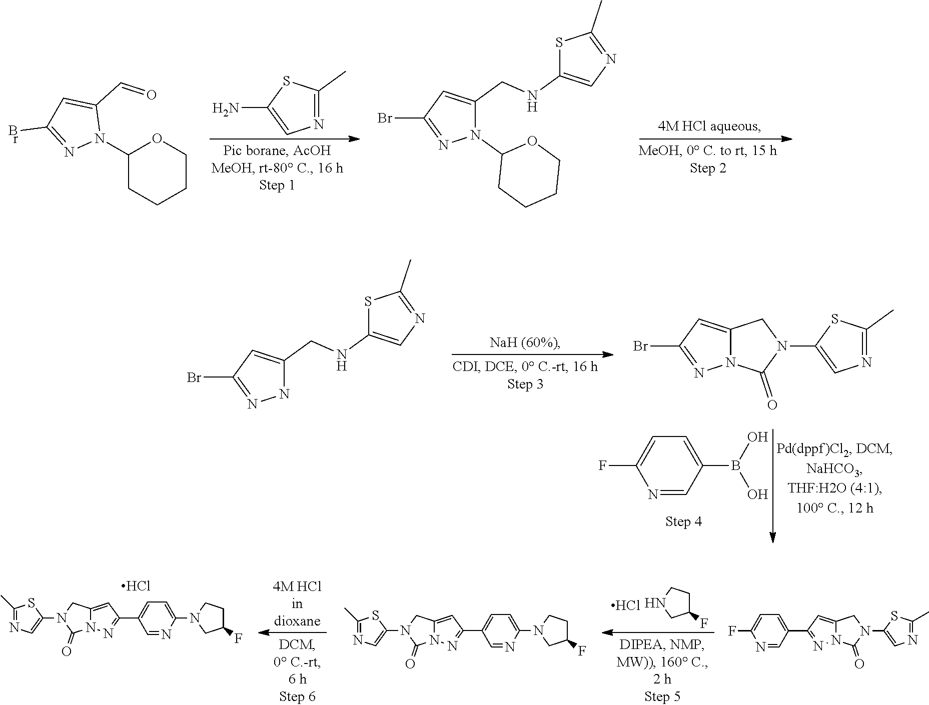

Step 1: To a solution of 3-bromo-1-(tetrahydro-2H-pyran-2-yl)-1H-pyrazole-5-carbaldehyde (1.0 g, 3.8 mmol) and 2-methylthiazol-5-amine (0.43 g, 3.8 mmol) in methanol (40 mL) was added glacial AcOH (0.02 mL, 0.38 mmol) at RT under N₂. Then the mixture was stirred for 15 min. After that pic borane (1.2 g, 11.5 mmol) was added and the mixture was refluxed at 80° C. for 16 h. The reaction mixture was concentrated under reduced pressure and the residue was quenched with sat. aq. NaHCO₃ solution at 0° C. and the product was extracted with 10% MeOH in DCM three times (50 mL×3). The extract was dried over Na₂SO₄ and concentrated under vacuum. The obtained crude mass was purified by column chromatography over basified silica gel (230-400 mesh) eluted in 80% EtOAc in hexane to afford N-((3-bromo-1-(tetrahydro-2H-pyran-2-yl)-1H-pyrazol-5-yl)methyl)-2-methylthiazol-5-amine as brownish liquid (0.54 g, 39%).

Step 2: To a stirred solution of N-((3-bromo-1-(tetrahydro-2H-pyran-2-yl)-1H-pyrazol-5-yl)methyl)-2-methylthiazol-5-amine (0.54 g, 1.51 mmol) in MeOH (13 mL) was added aq. 4M HCl (3.2 mL, 15.1 mmol) at 0° C. under N₂ atmosphere and stirred at RT for 15 h. The reaction mixture was cooled to 0° C. and quenched with saturated aq. NaHCO₃ until the resultant mixture pH reaches up to 8-9 and the product was extracted with 10% MeOH in DCM three times (25 mL×3). The combined organic layer was dried over Na₂SO₄ and concentrated under vacuum to afford N-((3-bromo-1H-pyrazol-5-yl)methyl)-2-methylthiazol-5-amine as yellow solid (0.26 g, 63%) directly used for next step without any further purification. MS (ESI): 275.00 [M+H]+.

Step 3: To an ice cool solution of N-((3-bromo-1H-pyrazol-5-yl)methyl)-2-methylthiazol-5-amine (260 mg, 0.95 mmol) in 1,2-DCE (3.9 mL) was added NaH (60% dispersed in mineral oil) (19 mg, 0.47 mmol) under N₂ atmosphere. Then, the mixture was stirred for 10 min. CDI (1.5 g, 9.5 mmol) was added to the reaction mixture and temperature was allowed to RT and stirred for 16 h. After completion of the reaction, the crude was quenched with ice cold water and the product was extracted with DCM three times (10 mL×3). The extract was dried over Na₂SO₄ and concentrated under vacuum. The residue was purified by silica gel chromatography (230-400 mesh) eluted in 3% MeOH in DCM to yield 2-bromo-5-(2-methylthiazol-5-yl)-4,5-dihydro-6H-imidazo[1,5-b]pyrazol-6-one as light brownish solid (135 mg, 47%).

¹H NMR (DMSO-d6) δ 7.5 (s, 1H), 6.73 (s, 1H), 5.04 (s, 2H), 2.61 (s, 3H).

LCMS: 300.65 [M+H]+

Step 4: In an oven-dried screw capped vial was added 2-bromo-5-(2-methylthiazol-5-yl)-4,5-dihydro-6H-imidazo[1,5-b]pyrazol-6-one (100 mg, 0.33 mmol), (6-fluoropyridin-3-yl)boronic acid (70 mg, 0.5 mmol), NaHCO₃ (140 mg, 1.6 mmol) and 1,4-dioxane (3.0 mL, 30 vol) under an argon atmosphere. The reaction mixture was degassed with argon for 15 min. Then, Pd(dppf)Cl₂·DCM (27 mg, 0.03 mmol) was added and again degassed for 10 min. The mixture was heated to 100° C. for 18 h. The reactants were consumed as monitored by TLC. After that the reaction mixture was quenched with ice-water and extracted with EtOAc three times (10 mL×3). The organic layer was dried over Na₂SO₄, concentrated and purified by silica gel chromatography (230-400 mesh) eluted in 3% MeOH in DCM to get 2-(6-fluoropyridin-3-yl)-5-(2-methylthiazol-5-yl)-4,5-dihydro-6H-imidazo[1,5-b]pyrazol-6-one as light brown solid (38 mg, 36%).

¹H NMR (DMSO-d6) δ 8.83 (d, 1H), 8.52 (td, 1H), 7.52 (s, 1H), 7.34 (dd, 1H), 7.15 (s, 1H), 5.11 (s, 2H), 2.63 (s, 3H).

LCMS: 316.2 [M+H]+

Step 5: 2-(6-fluoropyridin-3-yl)-5-(2-methylthiazol-5-yl)-4,5-dihydro-6H-imidazo[1,5-b]pyrazol-6-one (30 mg, 0.09 mmol), (R)-3-fluoropyrrolidine hydrogen chloride (17 mg, 0.14 mmol), DIPEA (0.04 mL, 0.28 mmol), and NMP (0.6 mL, 20 vol.) was taken in an oven-dried micro wave vial under argon atmosphere. The reaction mixture was heated under microwave irradiation at 160° C. for 2 h. The crude mixture was quenched with ice cold water (5 mL). The crude reaction mass was filtered through Buchner funnel and the obtained mass was washed with hexane three times (5 mL×3), dried under high vacuum to afford (R)-2-(6-(3-fluoropyrrolidin-1-yl)pyridin-3-yl)-5-(2-methylthiazol-5-yl)-4,5-dihydro-6H-imidazo[1,5-b]pyrazol-6-one as off-white solid (20 mg, 55%).

¹H NMR (500 MHz, DMSO-D6) δ 8.67 (d, 1H), 8.04 (dd, 1H), 7.48 (s, 1H), 6.94 (s, 1H), 6.62 (d, 1H), 5.47 (d, 1H), 5.07 (s, 2H), 3.87-3.64 (m, 3H), 3.55-3.42 (m, 2H), 2.62 (s, 3H), 2.33-2.10 (m, 2H).

LCMS: 384.8 [MH]+;

Step 6: To a stirred solution of (R)-2-(6-(3-fluoropyrrolidin-1-yl)pyridin-3-yl)-5-(2-methylthiazol-5-yl)-4,5-dihydro-6H-imidazo[1,5-b]pyrazol-6-one (20 mg, 0.052 mmol) in DCM (1.0 mL, 50 vol.) was added 4M HCl in 1,4-Dioxane (0.1 mL) at 0° C. under N₂ atmosphere and stirred at RT for 6 h. After completion of the reaction, solvent was evaporated, washed with pentane, dried under vacuum to afford (R)-2-(6-(3-fluoropyrrolidin-1-yl)pyridin-3-yl)-5-(2-methylthiazol-5-yl)-4,5-dihydro-6H-imidazo[1,5-b]pyrazol-6-one hydrogen chloride salt as brown solid (16 mg, 76%).

¹H NMR (500 MHz, DMSO-D6) δ 8.52 (s, 1H), 8.38 (s, 1H), 7.52 (s, 1H), 7.16-7.00 (m, 2H), 5.56 (d, 1H), 5.10 (s, 2H), 3.94-3.59 (m, 4H), 2.62 (s, 3H), 2.41-2.15 (m, 2H).

LCMS: 385.2 [M+H]+

### Example 19

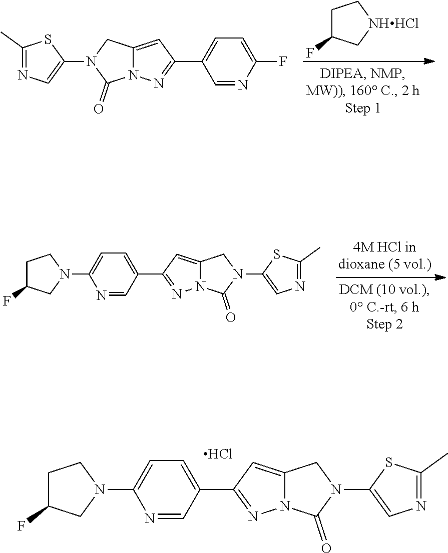

Step 1: 2-(6-fluoropyridin-3-yl)-5-(2-methylthiazol-5-yl)-4,5-dihydro-6H-imidazo[1,5-b]pyrazol-6-one (40 mg, 0.12 mmol), (S)-3-fluoropyrrolidine hydrogen chloride (23 mg, 0.19 mmol), DIPEA (0.06 mL, 0.38 mmol), and NMP (0.8 mL) was taken in an oven-dried micro wave vial under argon atmosphere. the reaction mixture was heated under microwave irradiation at 160° C. for 2 h. The reaction mixture was quenched with ice cold water (5 mL). The crude reaction mass was filtered through Büchner funnel and the obtained mass was washed with hexane three times (5 mL×3), dried under high vacuum to afford (S)-2-(6-(3-fluoropyrrolidin-1-yl)pyridin-3-yl)-5-(2-methylthiazol-5-yl)-4,5-dihydro-6H-imidazo[1,5-b]pyrazol-6-one as brown solid (16 mg, 33%).

¹H NMR (400 MHz, DMSO-D₆) δ 8.67 (s, 1H), 8.05 (d, 1H), 7.48 (s, 1H), 6.95 (s, 1H), 6.62 (d, 1H), 5.47 (d, 1H), 5.07 (s, 2H), 3.91-3.56 (m, 3H), 3.51-3.42 (m, 1H), 2.61 (s, 3H), 2.32-2.04 (m, 2H).

LCMS: 384.7 [M+H]+

Step 2: To a stirred solution of (S)-2-(6-(3-fluoropyrrolidin-1-yl)pyridin-3-yl)-5-(2-methylthiazol-5-yl)-4,5-dihydro-6H-imidazo[1,5-b]pyrazol-6-one (16 mg, 0.041 mmol) in DCM (0.8 mL, 50 vol.) was added 4M HCl in 1,4-Dioxane (0.08 mL, 5.0 vol.) at 0° C. under N₂ atmosphere and stirred at RT for 6 h. After completion of the reaction, solvent was evaporated, washed with pentane, dried under vacuum to afford (S)-2-(6-(3-fluoropyrrolidin-1-yl)pyridin-3-yl)-5-(2-methylthiazol-5-yl)-4,5-dihydro-6H-imidazo[1,5-b]pyrazol-6-one hydrogen chloride salt as brown solid (14 mg, 82%).

¹H NMR (400 MHz, DMSO-D6) δ 8.51 (d, 1H), 8.40 (d, 1H), 7.52 (s, 1H), 7.20-7.01 (m, 2H), 5.56 (d, 1H), 5.10 (s, 2H), 3.73-3.42 (m, 4H), 2.62 (s, 3H), 2.42-2.10 (m, 2H).

LCMS: 385.15 [M+H]+

### Example 20

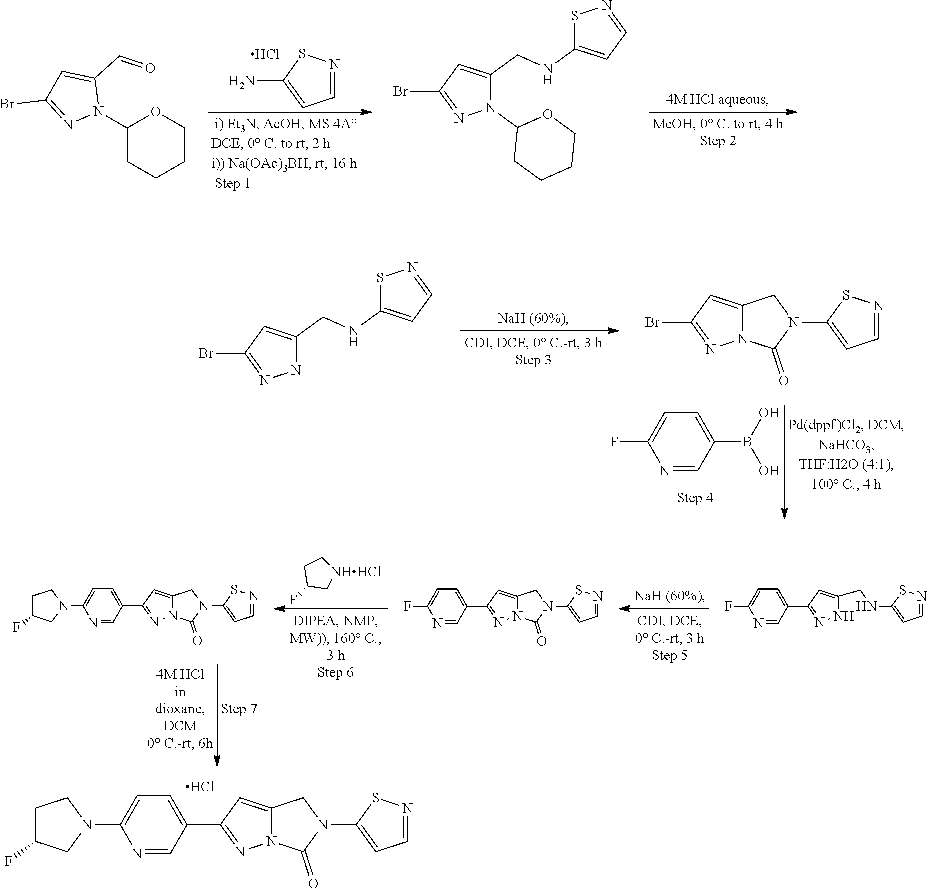

Step 1: To a stirred solution of 3-bromo-1-(tetrahydro-2H-pyran-2-yl)-1H-pyrazole-5-carbaldehyde (1.5 g, 5.8 mmol) and isothiazol-5-amine hydrogen chloride salt (1.0 g, 7.5 mmol) in 1,2 dichloro ethane (60 mL) was added triethyl amine (1.0 mL, 7.5 mmol) and was stirred at RT for 30 min. To this was added molecular sieves 4 A° and glacial AcOH (6.0 mL) under N₂, and kept for 2 h. Then, sodium triacetoxyborohydride (3.7 g, 17.3 mmol) was added and the mixture was stirred at RT for 16 h. The Progression of the reaction was monitored by TLC. The reaction mixture was quenched with aqueous saturated NaHCO₃ (30 mL) solution and the product was extracted with 5% MeOH in DCM three times (60 mL×3). The combined organic layer was dried over Na₂SO₄ and concentrated under vacuum. The obtained crude mass was purified by column chromatography over silica gel (100-200 mesh) eluted in 50% EtOAc in hexane to afford N-((3-bromo-1-(tetrahydro-2H-pyran-2-yl)-1H-pyrazol-5-yl) methyl) iso thiazol-5-amine as yellow solid (1.4 g, 70%). MS (ESI) 344.89 [M+H]+.

Step 2: To a stirred solution of N-((3-bromo-1-(tetrahydro-2H-pyran-2-yl)-1H-pyrazol-5-yl) methyl) iso thiazol-5-amine (1.4 g, 4.1 mmol) in MeOH (42 mL, 30 vol) was added aq. 4M HCl (10.2 mL) at 0° C. under N2 atmosphere and stirred at RT for 3 h. The reaction time was monitored by TLC. After completion, the reaction mixture was cooled to 0° C. and quenched with saturated aq. NaHCO₃ until the resultant mixture pH reaches up to 8-9. The solvent was removed under vacuum and the product was extracted with DCM three times (50 mL×3). The combined organic layer was dried over Na₂SO₄ and concentrated under vacuum. The obtained mass was washed with hexane three times (8 mL×3), dried under vacuum to afford N-((3-bromo-1H-pyrazol-5-yl)methyl)isothiazol-5-amine as yellow solid (700 mg, 66%) directly used for next step without any further purification. MS (ESI): 260.97 [M+H]+.

Step 3: To an ice cool solution of N-((3-bromo-1H-pyrazol-5-yl)methyl)isothiazol-5-amine (700 mg, 2.7 mmol) in 1,2-DCE (11 mL) was added NaH (60% dispersed in mineral oil) (54 mg, 1.3 mmol) under N2 atmosphere. Then, the mixture was allowed to RT and kept for 30 min. Then, CDI (4.38 g, 27 mmol) was added to the reaction mixture and stirred at RT for 3 h. After completion, the reaction mixture was quenched with ice cold water (3 mL). The crude reaction mass was filtered through Buchner funnel. The obtained mass was washed with hexane three times (5 mL×3), dried under high vacuum to afford 2-bromo-5-(isothiazol-5-yl)-4,5-dihydro-6H-imidazo[1,5-b]pyrazol-6-one as brown solid (500 mg, 65%).

¹H NMR (DMSO-d6) δ 8.38 (d, 1H), 7.16 (d, 1H), 6.77 (d, 1H), 5.11 (d, 2H).

MS (ESI): 286.98 [M+H]+

Step 4: In an oven-dried screw capped vial was added 2-bromo-5-(isothiazol-5-yl)-4,5-dihydro-6H-imidazo[1,5-b]pyrazol-6-one (100 mg, 0.35 mmol), boronic acid (100 mg, 0.7 mmol), NaHCO₃ (147 mg, 1.7 mmol), and dioxane:H2O (4:1, 4 mL) under an argon atmosphere. The reaction mixture was degassed with argon for 15 min. Then, Pd(dppf)Cl₂·DCM (57 mg, 0.07 mmol) was added and the mixture was heated to 100° C. for 4 h. The reactants were consumed as monitored by TLC. After that the reaction mixture was quenched with ice-water and extracted in 5% MeOH in DCM three times (10 mL×3). The organic layer was dried over Na₂SO₄, concentrated and purified by silica gel chromatography (100-200 mesh) eluted in 5% MeOH in DCM to get N-((3-(6-fluoropyridin-3-yl)-1H-pyrazol-5-yl)methyl)isothiazol-5-amine as brownish liquid (70 mg, 73%). MS (ESI): 276.11 [M+H]+.

Step 5: To an ice cool solution of N-((3-(6-fluoropyridin-3-yl)-1H-pyrazol-5-yl)methyl)isothiazol-5-amine (70 mg, 0.25 mmol) in 1,2-DCE (1.0 mL) was added NaH (60% dispersed in mineral oil) (5.0 mg, 0.13 mmol) under N₂ atmosphere. Then, the mixture was allowed to RT and kept for 30 min.

Then, CDI (405 mg, 2.5 mmol) was added to the reaction mixture and stirred at RT for 3 h. After completion, the reaction mixture was quenched with ice cold water and the crude reaction mass was filtered through Büchner funnel. The obtained mass was washed with hexane three times (5 mL×3), dried under high vacuum to afford 2-(6-fluoropyridin-3-yl)-5-(isothiazol-5-yl)-4,5-dihydro-6H-imidazo[1,5-b]pyrazol-6-one as brown solid (40 mg, 53%).

¹H NMR (DMSO-d6) δ 8.85 (d, 1H), 8.54 (m, 1H), 8.40 (d, 1H), 7.35 (dd, 1H), 7.18 (m, 2H), 5.18 (s, 2H).

LCMS: 301.80 [M]+

Step 6: 2-(6-fluoropyridin-3-yl)-5-(isothiazol-5-yl)-4,5-dihydro-6H-imidazo[1,5-b]pyrazol-6-one (40 mg, 0.13 mmol), (R)-3-fluoropyrrolidine hydrogen chloride (25 mg, 0.20 mmol), DIPEA (0.07 mL, 0.39 mmol) and NMP (0.5 mL) was taken in an oven-dried micro wave vial under argon atmosphere. The reaction mixture was heated under microwave irradiation at 160° C. for 3 h. After completion of the reaction, the crude mixture was quenched with ice cold water and the crude mass was filtered through Buchner funnel. The obtained mass was washed with hexane three times (5 mL×3), dried under high vacuum to afford (R)-2-(6-(3-fluoropyrrolidin-1-yl) pyridin-3-yl)-5-(isothiazol-5-yl)-4,5-dihydro-6H-imidazo[1,5-b]pyrazol-6-one as brown solid (17 mg, 35%).

¹H NMR (500 MHz, DMSO-D6) δ 8.69 (dd, 1H), 8.38 (d, 1H), 8.06 (dd, 1H), 7.13 (d, 1H), 6.99 (s, 1H), 6.62 (dd, 1H), 5.47 (d, 1H), 5.14 (s, 2H), 3.86-3.58 (m, 3H), 3.54-3.42 (m, 1H), 2.33-2.08 (m, 2H).

LCMS: 371.10 [M+H]+

Step 7: To a stirred solution of (R)-2-(6-(3-fluoropyrrolidin-1-yl) pyridin-3-yl)-5-(isothiazol-5-yl)-4,5-di hydro-6H-imidazo[1,5-b]pyrazol-6-one (17 mg, 0.045 mmol) in DCM (1.0 mL, 50 vol.) was added 4M HCl in 1,4-Dioxane (0.17 mL) at 0° C. under N₂ atmosphere and stirred at RT for 4 h. Then, the solvent was evaporated, washed with pentane, dried under vacuum to afford (R)-2-(6-(3-fluoropyrrolidin-1-yl) pyridin-3-yl)-5-(isothiazol-5-yl)-4,5-dihydro-6H-imidazo[1,5-b]pyrazol-6-one hydrogen chloride salt as brown solid (15 mg, 82%).

¹H NMR (400 MHz, DMSO-D6) δ 8.57 (d, 1H), 8.39 (d, 1H), 8.32 (d, 1H), 7.22-7.06 (m, 2H), 6.99 (s, 1H), 5.54 (d, 1H), 5.16 (d, 2H), 3.96-3.41 (m, 4H), 2.40-2.09 (m, 2H).

LCMS: 371.20 [M+H]+

### Example 21

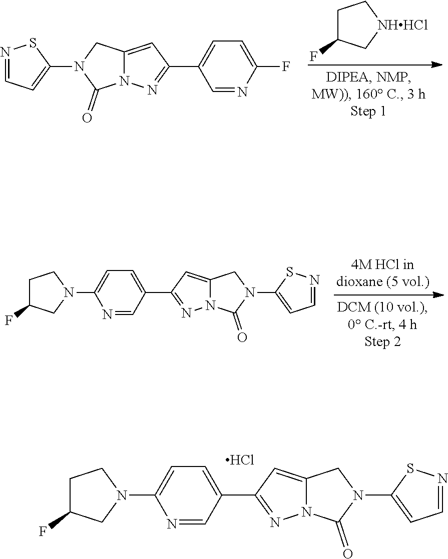

Step 1: 2-(6-fluoropyridin-3-yl)-5-(isothiazol-5-yl)-4,5-dihydro-6H-imidazo[1,5-b]pyrazol-6-one (50 mg, 0.17 mmol), (S)-3-fluoropyrrolidine hydrogen chloride (33 mg, 0.26 mmol), DIPEA (0.09 mL, 0.51 mmol) and NMP (0.5 mL) was taken in an oven-dried micro wave vial under argon atmosphere. The reaction mixture was heated at 160° C. for 3 h. Then, the reaction mixture was quenched with ice cold water and the crude reaction mass was filtered through Büchner funnel. The obtained mass was washed with hexane three times (3 mL×3), dried under high vacuum to afford (S)-2-(6-(3-fluoropyrrolidin-1-yl) pyridin-3-yl)-5-(isothiazol-5-yl)-4,5-dihydro-6H-imidazo[1,5-b]pyrazol-6-one as brown solid (25 mg, 40%).

¹H NMR (500 MHz, DMSO-D6) δ 8.69 (dd, 1H), 8.38 (d, 1H), 8.06 (dd, 1H), 7.13 (d, 1H), 7.00 (d, 1H), 6.62 (dd, 1H), 5.47 (d, 1H), 5.14 (s, 2H), 3.88-3.55 (m, 3H), 3.48 (td, 1H), 2.33-2.08 (m, 2H).

LCMS: 371.00 [M+H]+

Step 2: To a stirred solution of (S)-2-(6-(3-fluoropyrrolidin-1-yl) pyridin-3-yl)-5-(isothiazol-5-yl)-4,5-di hydro-6H-imidazo[1,5-b]pyrazol-6-one (25 mg, 0.07 mmol) in DCM (1.3 mL, 50 vol.) was added 4M HCl in 1,4-Dioxane (0.25 mL) at 0° C. under N₂ atmosphere and stirred at RT for 4 h. After completion of the reaction, solvent was evaporated, washed with pentane, dried under vacuum to afford (S)-2-(6-(3-fluoropyrrolidin-1-yl) pyridin-3-yl)-5-(isothiazol-5-yl)-4,5-dihydro-6H-imidazo[1,5-b]pyrazol-6-one hydrogen chloride salt as brown solid (26 mg, 93%).

¹H NMR (500 MHz, DMSO-D6) δ 8.56 (d, 1H), 8.48-8.29 (m, 2H), 7.25-7.11 (m, 2H), 7.04 (s, 1H), 5.55 (d, 1H), 5.17 (s, 2H), 3.96-3.59 (m, 4H), 2.39-2.14 (m, 2H).

LCMS: 371.15 [M+H]+

### Example 22

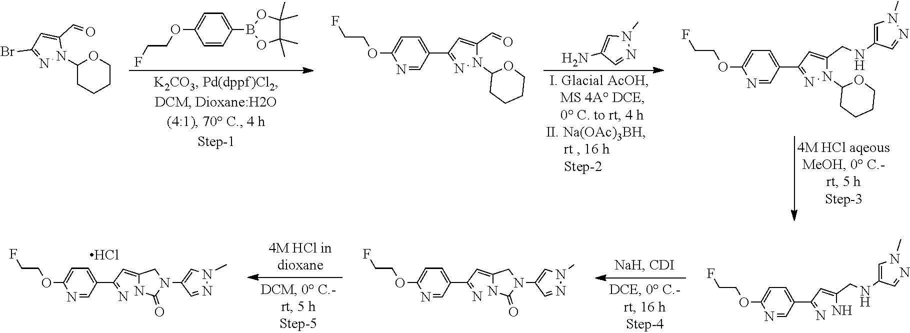

Step1: In an oven-dried screw capped vial was added 3-bromo-1-(tetrahydro-2H-pyran-2-yl)-1H-pyrazole-5-carbaldehyde (2.0 g, 7.7 mmol), boronic ester (4.12 g, 15.4 mmol), K₂CO₃ (2.13 g, 11.5 mmol) and dioxane:H2O (4:1, 50 mL) under an argon atmosphere. The reaction mixture was degassed with argon for 15 min. Then, Pd(dppf)Cl₂·DCM (630 mg, 0.77 mmol) was added and the mixture was heated to 70° C. for 4 h. After that the reaction mixture was quenched with ice-water and extracted with EtOAc three times (60 mL×3). The organic layer was dried over Na₂SO₄, concentrated and purified by silica gel chromatography (100-200 mesh) eluted in 25% EtOAc in Hexane to get 3-(6-(2-fluoroethoxy)pyridin-3-yl)-1-(tetrahydro-2H-pyran-2-yl)-1H pyrazole-5-carbaldehyde as off-white solid (2.0 g, 81%).

¹H NMR (CDCl3) δ 9.99 (s, 1H), 8.56 (dd, 1H), 8.10 (dd, 1H), 7.17 (s, 1H), 6.86 (m, 1H), 6.14 (dd, 1H), 4.83 (m, 1H), 4.71 (m, 1H), 4.65 (m, 1H), 4.58 (m, 1H), 4.07 (m, 1H), 3.76 (m, 1H), 2.51 (m, 1H), 2.11 (m, 2H), 1.71 (m, 4H), 1.34 (m, 1H), 1.24 (s, 2H).

MS (ESI): 320.24 (M+H)+

Step 2: To a stirred solution of 3-(6-(2-fluoroethoxy)pyridin-3-yl)-1-(tetrahydro-2H-pyran-2-yl)-1H pyrazole-5-carbaldehyde (800 mg, 2.5 mmol) and 1-methyl-1H-pyrazol-4-amine (320 mg, 3.2 mmol) in 1,2_dichloro ethane (32 mL) was added molecular sieves 4 A° and glacial AcOH (2.4 mL) under N₂, and kept for 4 h. Then, sodium triacetoxyborohydride (1.1 g, 5.0 mmol) was added and the mixture was stirred at RT for 16 h. The reaction mixture was quenched with aqueous saturated NaHCO₃ (80 mL) solution and the product was extracted with DCM (80 mL×3). The combined organic layer was dried over Na₂SO₄ and concentrated under vacuum. The obtained crude mass was purified by column chromatography over silica gel (100-200 mesh) eluted in 3% MeOH in DCM to afford N-((3-(6-(2-fluoroethoxy) pyridin-3-yl)-1-(tetrahydro-2H-pyran-2-yl)-1H-pyrazol-5-yl) methyl)-1-methyl-1H-pyrazol-4-amine as brown liquid (650 mg, 65%). ¹H NMR (DMSO-d6) δ 8.48 (dd, 1H), 8.04 (dd, 1H), 7.08 (d, 1H), 6.96 (d, 1H), 6.87 (dd, 1H), 6.66 (s, 1H), 5.49 (dd, 1H), 4.83 (s, 1H), 4.78 (t, 1H), 4.66 (m, 1H), 4.52 (m, 1H), 4.45 (m, 1H), 4.09 (dd, 2H), 3.87 (d, 1H), 3.64 (s, 3H), 3.60 (m, 1H), 2.30 (m, 1H), 1.99 (m, 1H), 1.87 (m, 1H), 1.63 (dt, 1H), 1.52 (m, 2H), 1.24 (m, 1H).

Step 3: To a stirred solution of N-((3-(6-(2-fluoroethoxy) pyridin-3-yl)-1-(tetrahydro-2H-pyran-2-yl)-1H-pyrazol-5-yl) methyl)-1-methyl-1H-pyrazol-4-amine (650 mg, 1.6 mmol) in MeOH (16 mL) was added aq. 4M HCl (6.5 mL, 10 vol) at 0° C. under N₂ atmosphere and stirred at RT for 5 h. Then, the reaction mixture was cooled to 0° C. and quenched with saturated aq. NaHCO₃ until the resultant mixture pH reaches up to 8-9. The solvent was removed under vacuum and the product was extracted with EtOAc three times (80 mL×3). The combined organic layer was dried over Na₂SO₄ and concentrated under vacuum. The obtained mass was washed with hexane three times (5 mL×3), dried under vacuum to afford N-((3-(6-(2-fluoroethoxy) pyridin-3-yl)-1H-pyrazol-5-yl)methyl)-1-methyl-1H-pyrazol-4-amine as yellow solid (400 mg, 80%).

¹H NMR (DMSO-d6) δ 12.90 (m, 1H), 8.53 (d, 1H), 8.06 (m, 1H), 7.08 (s, 1H), 6.97 (s, 1H), 6.92 (m, 1H), 6.61 (s, 1H), 4.83 (m, 2H), 4.70 (t, 1H), 4.56 (t, 1H), 4.48 (t, 1H), 4.02 (m, 2H), 3.67 (s, 3H).

MS (ESI): 317.18 (M+H)+Step 4: To an ice cool solution of N-((3-(6-(2-fluoroethoxy) pyridin-3-yl)-1H-pyrazol-5-yl)methyl)-1-methyl-1H-pyrazol-4-amine (250 mg, 0.79 mmol) in 1, 2-DCE (15 mL) was added NaH (60% dispersed in mineral oil) (17 mg, 0.4 mmol) under N₂ atmosphere. Then, the mixture was allowed to warm up to RT and kept for 30 min. Then, CDI (1.2 g, 7.9 mmol) was added to the reaction mixture and stirred at RT for 16 h. The reaction mixture was quenched with ice cold water and the product was extracted with 5% MeOH in DCM three times (20 mL×3). The extract was dried over Na₂SO₄ and concentrated under vacuum. The residue was purified by silica gel chromatography (100-200 mesh) eluted in 3% MeOH in DCM to yield 2-(6-(2-fluoroethoxy) pyridin-3-yl)-5-(1-methyl-1H-pyrazol-4-yl)-4,5-dihydro-6H-imidazo[1,5-b]pyrazol-6-one as white solid (170 mg, 63%).

¹H NMR (500 MHz, DMSO-D6) δ 8.73 (d, 1H), 8.24 (dd, 1H), 8.03 (s, 1H), 7.67 (s, 1H), 7.08-6.91 (m, 2H), 4.95 (s, 2H), 4.87-4.67 (m, 2H), 4.66-4.50 (m, 2H), 3.87 (s, 3H).

LCMS: 342.8 (M)+

Step 5: To a stirred solution of 2-(6-(2-fluoroethoxy) pyridin-3-yl)-5-(1-methyl-1H-pyrazol-4-yl)-4,5-dihydro-6H-imidazo[1,5-b]pyrazol-6-one (80 mg, 0.23 mmol) in DCM (4 mL) was added 4M HCl in 1,4-Dioxane (0.8 mL) at 0° C. under N₂ atmosphere and stirred at RT for 5 h. After completion of the reaction, solvent was evaporated, washed with pentane, dried under vacuum to afford 2-(6-(2-fluoroethoxy) pyridin-3-yl)-5-(1-methyl-1H-pyrazol-4-yl)-4,5-dihydro-6H-imidazo[1,5-b]pyrazol-6-one hydrogen chloride salt as white solid (80 mg, 91%).

¹H NMR (400 MHz, DMSO-D6) δ 8.73 (dd, 1H), 8.25 (dd, 1H), 8.04 (d, 1H), 7.67 (d, 1H), 7.05-6.94 (m, 2H), 4.96 (d, 2H), 4.89-4.66 (m, 2H), 4.66-4.44 (m, 2H), 3.87 (s, 3H).

LCMS: 342.8 (M+H)+

### Example 23

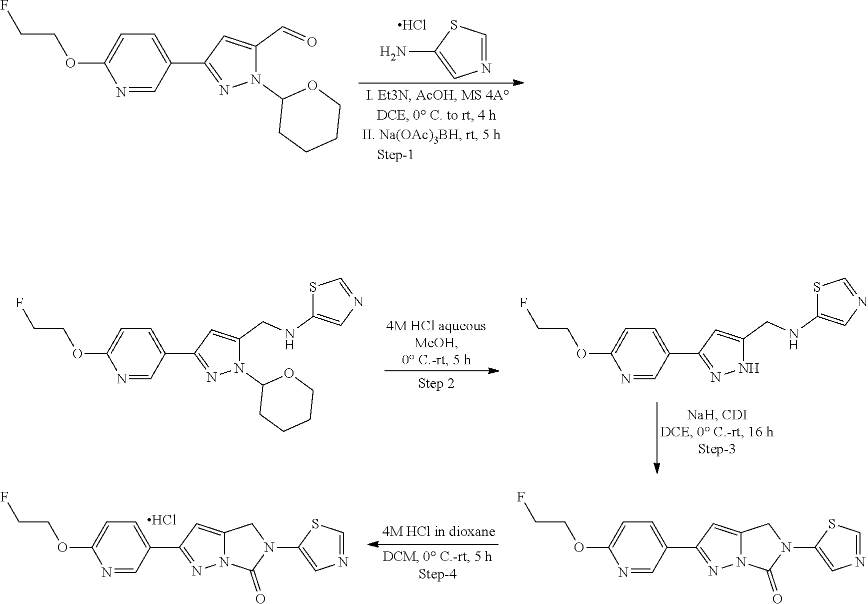

Step 1: To a stirred solution of (3-(6-(2-fluoroethoxy) pyridin-3-yl)-1-(tetrahydro-2H-pyran-2-yl)-1H-pyrazole-5-carbaldehyde (0.8 mg, 2.5 mmol) and thiazol-5-amine hydrogen chloride salt (0.445 g, 3.26 mmol) in 1,2 dichloro ethane (32 mL) was added triethyl amine (0.45 mL, 3.26 mmol) and was stirred at RT for 30 min. To this was added molecular sieves 4 A° and glacial AcOH (3.2 mL) under N₂, and kept for 4 h. Then, sodium triacetoxyborohydride (1.06 g, 5.02 mmol) was added and the mixture was stirred at RT for 5 h. The Progression of the reaction was monitored by TLC. The reaction mixture was quenched with aqueous saturated NaHCO₃ (50 mL) solution and the product was extracted with DCM three times (60 mL×3). The combined organic layer was dried over Na₂SO₄ and concentrated under vacuum. The obtained crude mass was purified by column chromatography over silica gel (100-200 mesh) eluted in 3% MeOH in DCM to afford N-((3-(6-(2-fluoroethoxy) pyridin-3-yl)-1-(tetrahydro-2H-pyran-2-yl)-1H-pyrazol-5-yl) methyl) thiazol-5-amine as brown liquid (0.6 g, 59%).

¹H NMR (DMSO-d6) δ 8.48 (dd, 1H), 8.15 (s, 1H), 8.08 (m, 1H), 6.94 (s, 1H), 6.88 (dd, 1H), 6.79 (q, 1H), 6.75 (s, 1H), 5.76 (s, 2H), 5.53 (m, 1H), 4.80 (t, 1H), 4.71 (t, 1H), 4.55 (t, 1H), 4.49 (t, 1H), 4.36 (m, 2H), 3.90 (d, 1H), 3.66 (td, 1H), 2.32 (m, 1H), 1.96 (m, 3H), 1.66 (m, 1H), 1.59 (s, 3H).

MS (ESI): 402.20 (M−H)+

Step 2: To a stirred solution of N-((3-(6-(2-fluoroethoxy) pyridin-3-yl)-1-(tetrahydro-2H-pyran-2-yl)-1H-pyrazol-5-yl) methyl) thiazol-5-amine (200 mg, 0.5 mmol) in MeOH (6 mL, 30 vol.) was added aq. 4M HCl (1.3 mL, 10 vol) at 0° C. under N₂ atmosphere and stirred at RT for 5 h. The reaction time was monitored by TLC. The reaction mixture was cooled to 0° C. and quenched with saturated aq. NaHCO₃ until the resultant mixture pH reaches up to 8-9. The solvent was removed under vacuum and the product was extracted with EtOAc three times (50 mL×3). The combined organic layer was dried over Na₂SO₄ and concentrated under vacuum. The obtained mass was washed with hexane three times (5 mL×3), dried under vacuum to afford N-((3-(6-(2-fluoroethoxy) pyridin-3-yl)-1H-pyrazol-5-yl) methyl) thiazol-5-amine as brown solid (100 mg, 63%). MS (ESI): 320.08 (M+H)+

Step 3: To an ice cool solution of N-((3-(6-(2-fluoroethoxy) pyridin-3-yl)-1H-pyrazol-5-yl) methyl) thiazol-5-amine (100 mg, 0.3 mmol) in 1,2-DCE (6 mL) was added NaH (60% dispersed in mineral oil) (6.0 mg, 0.16 mmol) under N₂ atmosphere. Then, the mixture was allowed to RT and kept for 30 min. Then, CDI (500 mg, 3.1 mmol) was added to the reaction mixture and stirred at RT for 16 h. After completion, the reaction mixture was quenched with ice cold water and the product was extracted with 5% MeOH in DCM three times (30 mL×3). The extract was dried over Na₂SO₄ and concentrated under vacuum. The residue was purified by silica gel chromatography (100-200 mesh) eluted in 3% MeOH in DCM to yield 2-(6-(2-fluoroethoxy) pyridin-3-yl)-5-(thiazol-5-yl)-4,5-dihydro-6H-imidazo [1,5-b] pyrazol-6-one as white solid (40 mg, 36%).

¹H NMR (400 MHz, DMSO-D6) δ 8.81 (d, 1H), 8.75 (dd, 1H), 8.27 (dd, 1H), 7.79 (d, 1H), 7.08 (s, 1H), 7.01 (dd, 1H), 5.15 (s, 2H), 4.89-4.67 (m, 2H), 4.67-4.47 (m, 2H).

LCMS: 345.9 (M)+

Step 4: To a stirred solution of 2-(6-(2-fluoroethoxy) pyridin-3-yl)-5-(thiazol-5-yl)-4,5-dihydro-6H-imidazo [1,5-b] pyrazol-6-one (40 mg, 0.12 mmol) in DCM (2.4 mL) was added 4M HCl in 1,4-dioxane (0.4 mL) at 0° C. under N2 atmosphere and stirred at RT for 5 h. Then, solvent was evaporated, washed with pentane, dried under vacuum to afford 2-(6-(2-fluoroethoxy) pyridin-3-yl)-5-(thiazol-5-yl)-4,5-dihydro-6H-imidazo [1,5-b] pyrazol-6-one hydrogen chloride salt as white solid (43 mg, 98%). ¹H NMR (400 MHz, DMSO-D6) δ 8.81 (d, 1H), 8.75 (dd, 1H), 8.27 (dd, 1H), 7.80 (d, 1H), 7.08 (s, 1H), 7.01 (dd, 1H), 5.15 (s, 2H), 4.89-4.69 (m, 2H), 4.65-4.48 (m, 2H).

LCMS: 345.7 (M)+

### Radioligand Synthesis

### Example-1 [³H-1]

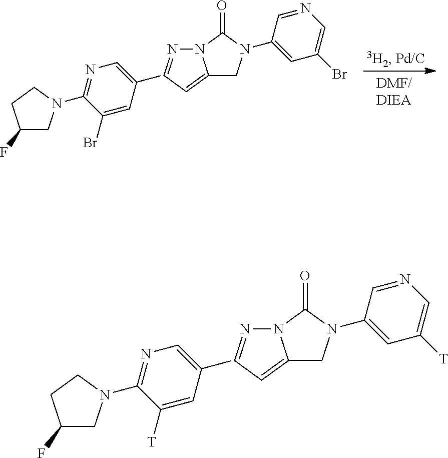

Precursor 1 (0.5 mg) was dissolved in dimethylformamide (DMF) (0.3 mL) and N,N-diisopropylethylamine (DIEA) (5 μL) in a tritium reaction vessel. 10% Pd/C (0.5 mg) was added and the vessel was pressurized to 0.5 atm with tritium gas at −200° C. The solution was stirred for 1 h at room temperature, cooled to −200° C. and excess gas was removed. The reaction flask was rinsed with 4×1 mL CH₃OH, passing each of the CH₃OH washes through a celite pad. The combined methanol was removed under vacuum. The material was purified by HPLC. The mobile phase was removed and the product was redissolved in absolute ethanol. (10 mCi with a radiochemical purity of >99% and a specific activity of 54.8 Ci/mmol). T means Tritium (³H). MS (ESI): m/z=369 (100%) [M+H]⁺

### Biological Assay Description and Corresponding Results

1. Preparation of Human Parkinson's Disease (PD) Brain-Derived Alpha-Synuclein (a-Syn) Aggregates

The procedure was adapted from the protocol described in Spillantini et al., 1998. Frozen tissue blocks from PD donors were thawed on ice and homogenized using a glass dounce homogenizer. The homogenate was then centrifuged at 11,000×g (12,700 RPM) in an ultracentrifuge (Beckman, XL100K) for 20 minutes at 4° C. using a pre-cooled 70.1 rotor (Beckman, 342184). Pellets were resuspended in extraction buffer [10 mM Tris-HCl pH 7.4, 10% sucrose, 0.85 mM NaCl, 1% protease inhibitor (Calbiochem 539131), 1 mM EGTA, 1% phosphatase inhibitor (Sigma P5726 and P0044)] and centrifuged at 15,000×g (14,800 RPM, a 70.1 Ti rotor) for 20 minutes at 4° C. Pellets were discarded and sarkosyl (20% stock solution, Sigma L7414) was added to the supernatants to a final concentration of 1% at room temperature for one hour. This solution was then centrifuged at 100,000×g (38,000 RPM, 70.1 Ti rotor) for one hour at 4° C. Pellets containing enriched alpha-synuclein aggregates were resuspended in PBS and stored at −80° C. until use.

### 2. Micro-Radiobinding Competition Assay for the Determination of Binding Affinity

PD brain-derived alpha-synuclein aggregates were spotted onto microarray slides. The slides were incubated with [³H]-alpha-synuclein reference at 6 nM or 20 nM and the example compounds (non-radiolabelled) at 1 μM and 100 nM. In some cases, the non-radiolabelled example compounds were further assessed for a range of different concentrations, varying from 0.05 nM to 2 μM. After incubation, slides were washed and scanned by a real-time autoradiography system (BeaQuant, ai4R). Quantification of the signal was performed by using the Beamage image analysis software (ai4R). Non-specific signal was determined with an excess of non-radiolabelled Example-1 (2 μM) and specific binding was calculated by subtracting the non-specific signal from the total signal. Competition was calculated as percent, where 0% was defined as the specific binding in the presence of vehicle and 100% as the values obtained in the presence of excess of the non-radiolabelled Example-1. Kᵢ values were calculated in GraphPad Prism7 by applying a nonlinear regression curve fit using a one site, specific binding model. All measurements were performed with at least two technical replicates. For compounds tested in more than one experiment, the mean of the replicates or Kᵢ values in independent experiments is reported.

Results: Example compounds were assessed for their potency to compete with the binding of [³H]-reference alpha-synuclein ligand to PD patient brain-derived alpha-synuclein aggregates. Results of the micro-radiobinding competition assay for the example compounds tested are shown in Table 3 as: % competition at 1 μM and 100 nM. The Table 3 also shows K; values.

TABLE 3

| Example | Micro-radiobinding competition assay | | |
|---|---|---|---|
| Compound | Competition at | Competition at | |
| no. | 1 μM (%) | 100 nM (%) | Ki (nM) |
| 1 | 91 | 56 | 69 |
| 2 | 97 | 87 | *46 |
| 3 | 89 | 74 | |
| 4 | 100 | 94 | |
| 5 | 89 | 59 | 26 |
| 6 | 93 | 43 | |
| 7 | 96 | 73 | |
| 8 | 73 | 27 | 64 |
| 9 | 62 | 45 | |
| 10 | 100 | 72 | |
| 11 | 76 | 25 | 252 |
| 12 | 90 | 76 | 34 |
| 13 | 71 | 55 | 101 |
| 14 | 85 | 56 | 80 |
| 15 | 80 | 48 | 141 |
| 16 | 98 | 79 | |
| 17 | 95 | 66 | |
| 18 | 83 | 51 | 51 |
| 19 | 78 | 41 | 65 |
| 20 | 87 | 85 | |
| 21 | 90 | 82 | |
| 22 | 89 | 45 | |
| 23 | 82 | 31 | |

Table 3: Assessment of binding affinity by micro-radiobinding competition assay on human PD brain-derived alpha-synuclein aggregates. Percent (%) competition over the tritiated [³H]-Example-1 ligand in the presence of 1 μM and 100 nM of example compounds 1-9. Kᵢ values are also shown for selected example compounds. *, mean of Ki values in two independent experiments using PD brain-derived homogenates from two different donors. As shown in Table 3, example compounds 1-9 of the present invention show potent binding to PD brain-derived alpha-synuclein aggregates.

### 3. Assessment of Target Engagement in Alpha-Synucleinopathies and AD Tissues

### 3 A: By High Resolution Micro-Autoradiography

The protocol was adapted from Marquie et al., 2015. Sections were incubated with tritiated example compound 1 ([³H]-Example-1) at 10 nM or 20 nM or a reference Tau ligand ([³H]-Tau-Ref at 20 nM for one hour at room temperature (RT). Sections were then washed as follows: One time in ice-cold 50 mM Tris-HCl pH 7.4 buffer for one minute, two times in 70% ice-cold ethanol for one minute, one time in ice-cold 50 mM Tris-HCl pH 7.4 buffer for one minute and finally rinsed briefly in ice-cold distilled water. Sections were subsequently dried and then exposed to Ilford Nuclear Emulsion Type K5 (Agar Scientific, AGP9281) in a light-proof slide storage box. After five days, the sections were developed by immersing them successively in the following solutions: 1.) Ilford Phenisol Developer (1:5 dilution in H₂O, Agar Scientific, AGP9106), 2.) Ilfostop solution (1:20 dilution in H₂O, Agar Scientific, AGP9104), 3.) Ilford Hypam Fixer (1:5 dilution in H₂O, Agar Scientific, AGP9183) and finally rinsed with H₂O.

When indicated, immunostaining was also performed on the same section. For image acquisition, sections were mounted using ProLong Gold Antifade reagent (Invitrogen P36930) and imaged on a Panoramic150 Slide Scanner (3DHistech) with a 20×objective capturing separately brightfield and fluorescent images.

### 3B. By Staining of Sections Using Antibodies

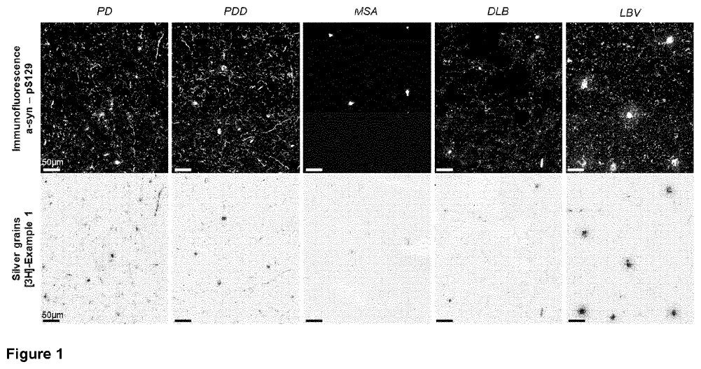

Brain sections were immunostained using a commercially available antibody, specific for phosphorylated serine at amino acid 129 alpha-synuclein (a-syn-pS129, rabbit monoclonal, Abcam 51253). Sections were fixed for 15 minutes at 4° C. with 4% formaldehyde (Sigma, 252549) and washed three times for five minutes with 1×PBS (Dulbecco's phosphate buffered saline, Sigma D1408) at RT. Next, sections were saturated and permeabilized in blocking buffer (PBS, 10% NGS, 0.25% Triton X-100) for one hour at RT and incubated overnight at 4° C. with the primary antibody corresponding to a-syn-pS129 (in PBS, 5% NGS, 0.25% Triton X-100). The following day, sections were washed three times for five minutes with 1×PBS before incubation with a secondary, AlexaFluor647-labelled goat-anti-rabbit (Abcam, ab150079) antibody for 45 minutes at RT. Following incubation with secondary antibody the sections were washed three times in PBS before being processed further. For image acquisition, sections were mounted using ProLong Gold Antifade reagent (Invitrogen P36930) and imaged with a Panoramic150 Slide Scanner (3DHistech; Hungary). Results: High-resolution micro-autoradiography with [³H]-Example-1 was performed on frozen human brain sections from different alpha-synucleinopathy cases. Strong autoradiography signal from [³H]-Example-1 was detected in the form of accumulating silver grains (FIG. 1 bottom) and co-localized with immunofluorescence signal from a-syn-pS129 antibody (FIG. 1 top) suggesting strong target engagement on Lewy bodies and Lewy neurites, as well as alpha-synuclein aggregates of very small size in PD and other alpha-synucleinopathies, including Multiple System Atrophy (MSA), Dementia with Lewy bodies (DLB), Lewy Body Variant of Alzheimer's disease (LBV) and Parkinson's disease dementia (PDD).

4. Assessment of Specific Binding in Brain Sections from PD, PDD, MSA, LBV and Non-Demented Control (NDC) Donors by Autoradiography

Frozen human brain sections from one familial PD case (alpha-synuclein [SNCA] gene G51D missense mutation), labelled as SNCA (G51D), one PDD case, one MSA case, one LBV case and two non-demented control (NDC) cases were first briefly fixed for 15 minutes at 4° C. with 4% paraformaldehyde (Sigma, 252549) and washed three times for five minutes with PBS (Dulbecco's phosphate buffered saline, Sigma) at RT. All slides were then equilibrated for 20 minutes in 50 mM Tris-HCl pH 7.4 buffer prior to use in the experiment. Each brain section was incubated with a fixed concentration (10 nM) of tritiated example compound 1 ([³H]-Example-1) or increasing concentrations of [³H]-Example-1 in the range of 2.5 nM to 80 nM of tritiated compound in Tris-HCl buffer for two hours at RT (Total binding, ‘TB’). To determine non-specific (NSB) binding ([³H]-Example-1 was mixed with 5 μM of non-radiolabelled compound (Example 1, self-block, ‘NSB’). The slides were washed and then exposed and scanned in a real-time autoradiography system (BeaQuant instrument, ai4R). Specific binding was determined by subtracting the non-specific signal from the total signal. K_{d} values were calculated in GraphPad Prism7 by applying a nonlinear regression curve fit using a one site specific binding model.

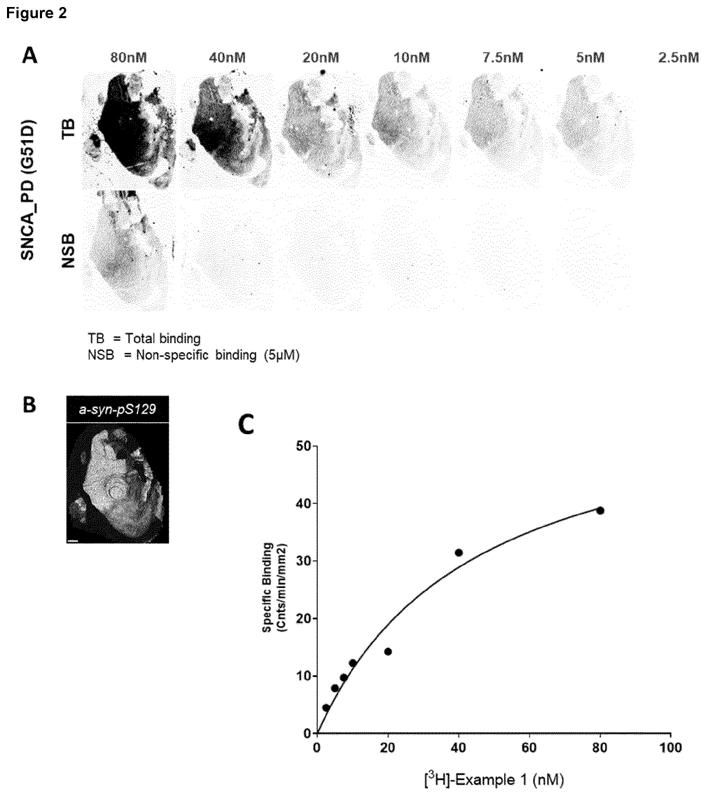

Results: [³H]-Example-1 displayed a dose-dependent autoradiography signal in a genetic PD case (FIG. 2A). The displaceable signal correlated well with the localization of alpha-synuclein pathology, as determined by staining with a-syn-pS129 antibody, indicating specific binding of the compound to PD tissue (FIG. 2B). By quantifying the specific signal, the dissociation constant (K_{d}) was calculated at 44 nM (FIG. 2C/Table 4), suggesting good binding affinity to pathological alpha-synuclein aggregates.

TABLE 4
[³H]-Example-1 in genetic PD (SNCA (G51D))

| Kd | 44 nM |
|---|---|
| R² | 0.97 |

Table 4: Assessment of binding affinity of [³H]-Example-1 on human brain tissue sections from a familial PD case (G51D missense mutation) by autoradiography. The dissociation constant (K_{d}) was calculated by applying a nonlinear regression curve fit using a one site, specific binding model in GraphPad Prism7. R² is the coefficient of determination.

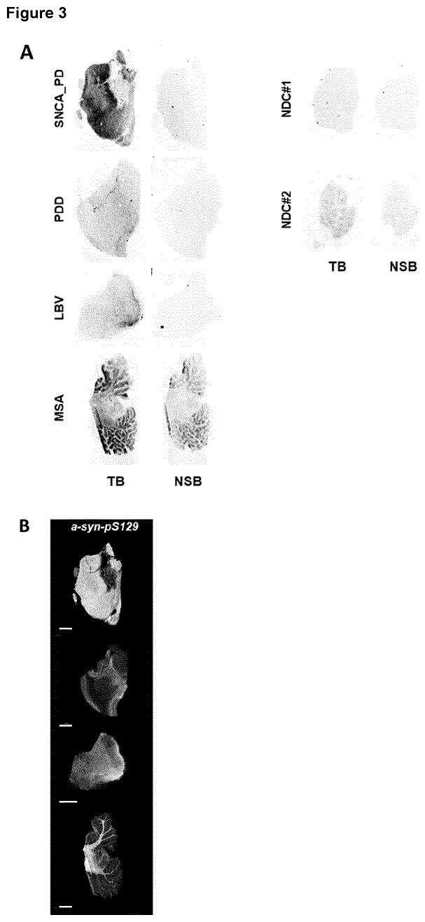

Additionally, [³H]-Example-1 displayed target engagement in various alpha-synucleinopathy tissues, including one PDD, one LBV and one MSA case (FIG. 3A). The displaceable signal correlated well with the localization and load of alpha-synuclein pathology, as determined by staining with a-syn-pS129 antibody (FIG. 3B), indicating specific binding of the compound. Furthermore, the autoradiographic signal appeared greater in diseased donors compared to non-demented control cases, for which signal was very weak (FIG. 3A).

### 5. Saturation Binding Studies on PD Brain-Derived Alpha-Synuclein Aggregates by Micro-Radiobinding

PD brain-derived alpha-synuclein aggregates were spotted onto microarray slides. The slides were incubated with [³H]-Example-1 at increasing concentrations in the range of 156 μM to 47 nM. After incubation, slides were washed and exposed to a phosphor storage screen (GE healthcare, BAS-IP TR 2025). Following exposure, phosphor storage screens were scanned with a laser imaging system (Typhoon FLA 7000) to readout the signal from the radiobinding experiments described above. Quantification of the signal was performed using the ImageJ software package. Non-specific signal was determined with an excess of non-radiolabelled reference ligand (Example-1 at 2 μM) and specific binding was calculated by subtracting the non-specific signal from the total signal. K_{d} values were calculated in GraphPad Prism7 by applying a nonlinear regression curve fit using a one site specific binding model.

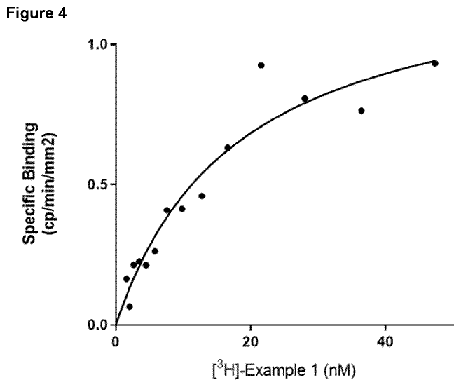

Results: [³H]-Example-1 was assessed in saturation binding studies on PD tissue homogenates by micro-radiobinding. As shown in FIG. 4 and Table 5, the dissociation constant (Kd) was calculated at 18 nM, suggesting good binding affinity to pathological alpha-synuclein aggregates.

TABLE 5
[³H]-Example-1in Genetic PD (SNCA (G51D))

| Kd | 18 nM |
|---|---|
| R² | 0.93 |

Table 5: Assessment of binding affinity of [³H]-Example-1 on human brain tissue homogenates from an idiopathic PD case by micro-radiobinding. The dissociation constant (K_{d}) was calculated by applying a nonlinear regression curve fit using a one site, specific binding model in GraphPad Prism7. R² is the coefficient of determination.

### 6. Radiobinding Competition Assay for Determination of Inhibitor Constant (Ki) on AD Brain Homogenates

### Preparation of Human Alzheimer's Disease (AD) Brain Homogenates:

The procedure was adapted from the protocol described in Bagchi et al., 2013. Frozen tissue blocks from AD donors were thawed on ice and homogenized in high salt buffer (50 mM Tris-HCl pH 7.5, 0.75M NaCl, 5 mM EDTA) supplemented with protease inhibitors (Complete; Roche 11697498001) at 4° C. using a glass Dounce homogenizer. The homogenate was centrifuged at 100,000×g (38,000 RPM) in an ultracentrifuge (Beckman, XL100K) for one hour at 4° C. using a pre-cooled 70.1 rotor (Beckman, 342184). Pellets were resuspended in high salt buffer supplemented with 1% Triton X-100 and homogenized at 4° C. using a glass Dounce homogenizer. The homogenates were centrifuged again at 100,000×g (38,000 RPM, 70.1 rotor) for one hour at 4° C. Pellets were resuspended in high salt buffer supplemented with 1% Triton X-100 and 1M sucrose and homogenized at 4° C. using a glass Dounce homogenizer. The homogenates were centrifuged at 100,000×g (38,000 RPM, 70.1 rotor) for one hour at 4° C. The resulting pellets containing the insoluble fraction were resuspended in PBS, aliquoted and stored at −80° C. until use.

A fixed concentration of AD insoluble fraction was incubated with a tritiated reference Abeta ligand ([³H]-Abeta-Ref) at 10 nM and increasing concentrations of non-radiolabelled example compound 1 in the range of 400 μM to 2 μM for two hours at RT. The samples were then filtered under vacuum in GF/C filter plates (PerkinElmer) to trap the aggregates with the bound radioligand and washed five times with 50 mM Tris pH 7.5. The GF/C filters were then dried and scintillation liquid (UltimateGold, PerkinElmer) was added in each well. The filters were analyzed on a Microbeta2 scintillation counter (PerkinElmer). Non-specific signal was determined with an excess of non-radiolabelled reference ligand (2 μM) and specific binding was calculated by subtracting the non-specific signal from the total signal. Competition was calculated as percent, where 0% was defined as the specific binding in the presence of vehicle and 100% as the values obtained in the presence of excess of the non-radiolabelled reference ligand. Kᵢ values were calculated in GraphPad Prism7 by applying a nonlinear regression curve fit using a one site, specific binding model. Measurements were performed with at least two replicates in two independent experiments.

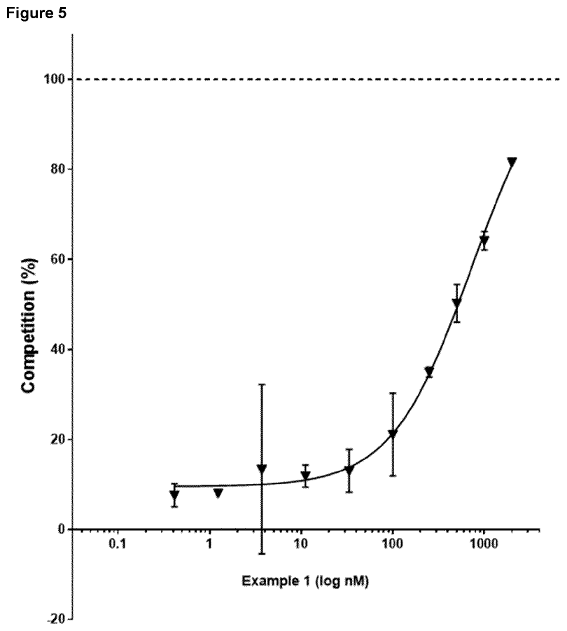

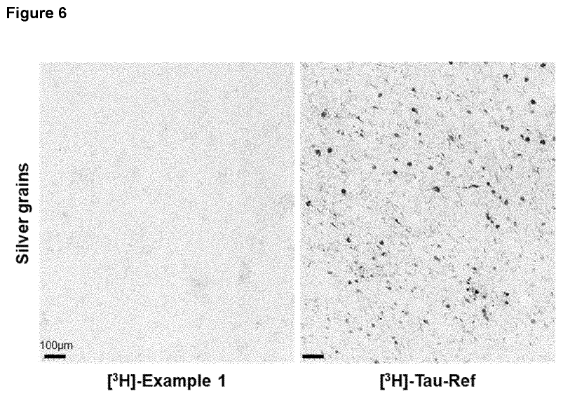

Results: As shown in FIG. 5 and Table 6, the Ki value of example compound 1 in AD brain-derived homogenates was determined at 360 nM. Based on the binding affinity of [³H]-Example-1 on PD brain tissue by autoradiography and in PD brain homogenates by micro-radiobinding, example compound 1 showed good selectivity for alpha-synuclein over Abeta pathological aggregates present in the human AD brain homogenates. Additionally, [³H]-Example-1 did not display specific target engagement on Tau aggregates in AD brain tissue, as compared to a reference Tau binder used as a positive control (FIG. 6), suggesting good selectivity for alpha-synuclein over Tau pathological aggregates. Overall, these data indicate the selectivity for alpha-synuclein of example compound 1 over other amyloid-like proteins such as Abeta and Tau.

TABLE 6
Example-1

| Ki | 360 nM |
|---|---|
| R² | 0.96 |

Table 6: Ki value determination of example compound 1 for the displacement of [³H]-Abeta-Ref with non-radiolabelled example compound 1 on AD brain-derived homogenates. Kᵢ, and R² values were calculated by applying a nonlinear regression curve fit using a one site, specific binding model in GraphPad Prism7.

1. A compound of formula (I): 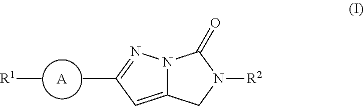
or a detectably labelled compound, stereoisomer, racemic mixture, pharmaceutically acceptable salt, hydrate, or solvate thereof, wherein 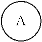
is a 6-membered heteroaryl which is optionally substituted with at least one substituent independently selected from halo, or C₁-C₄alkyl;
R¹ is halo, haloC₁-C₄alkoxy, or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl.

2. The compound of formula (I) according to claim 1: 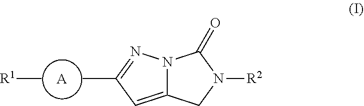
or a detectably labelled compound, stereoisomer, racemic mixture, pharmaceutically acceptable salt, hydrate, or solvate thereof, wherein 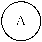
is a 6-membered heteroaryl;
R¹ is halo, or a 4- to 6-membered heterocyclyl which is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl.

3. The compound according to claim 1, having a formula (IIa), (IIb), (IIb′), (IIc), (IId) or (IIe): 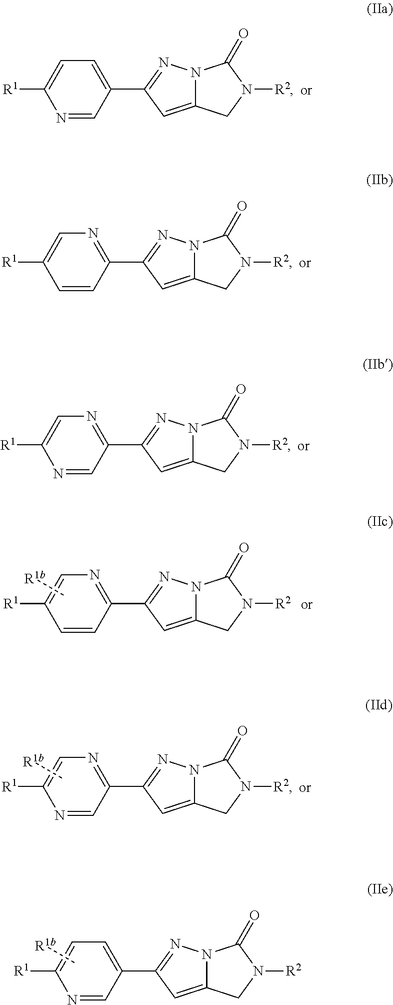
or a detectably labelled compound, stereoisomer, racemic mixture, pharmaceutically acceptable salt, hydrate, or solvate thereof, wherein R^{1b} is halo or C₁-C₄alkyl.

4. The compound according to claim 1, wherein R¹ is a 4- to 6-membered heterocyclyl selected from the following: 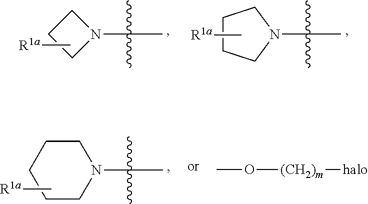
wherein R^{1a} is halo or H, and m is 1 or 2: wherein R¹ is preferably a 5-membered heterocyclyl selected from the following:

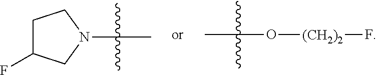

5. (canceled)

6. The compound according to claim 1, wherein R² is a 5-membered or 6-membered heteroaryl selected from the following: 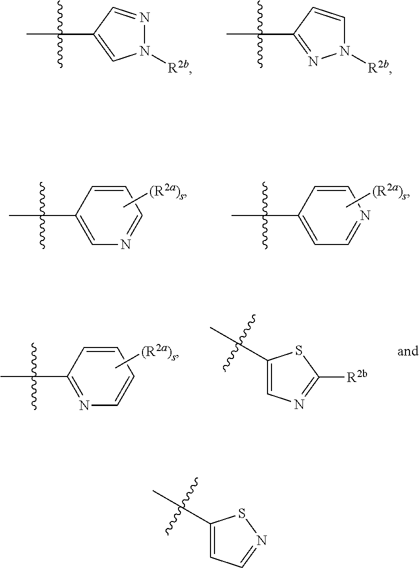
wherein
R^{2a} is independently selected from haloC₁-C₄alkyl, haloC₁-C₄alkoxy, C₁-C₄alkoxy, and C₁-C₄alkyl;
R^{2b} is selected from H, haloC₁-C₄alkyl, haloC₁-C₄alkoxy, C₁-C₄alkoxy, and C₁-C₄alkyl; and
s is 0, 1 or 2; wherein R² is preferably a 5-membered or 6-membered heteroaryl selected from the following: 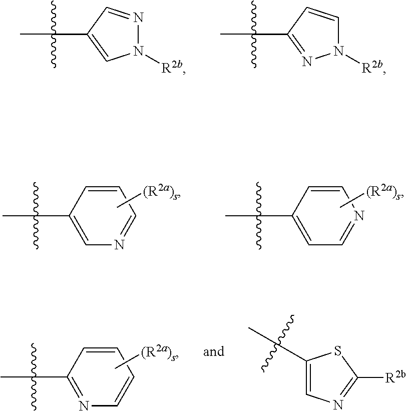
wherein
R^{2b} is selected from H, C₁-C₄alkyl, and haloC₁-C₄alkyl; and
s is 0.

7. (canceled)

8. The compound according to claim 1, wherein the compound is selected from: 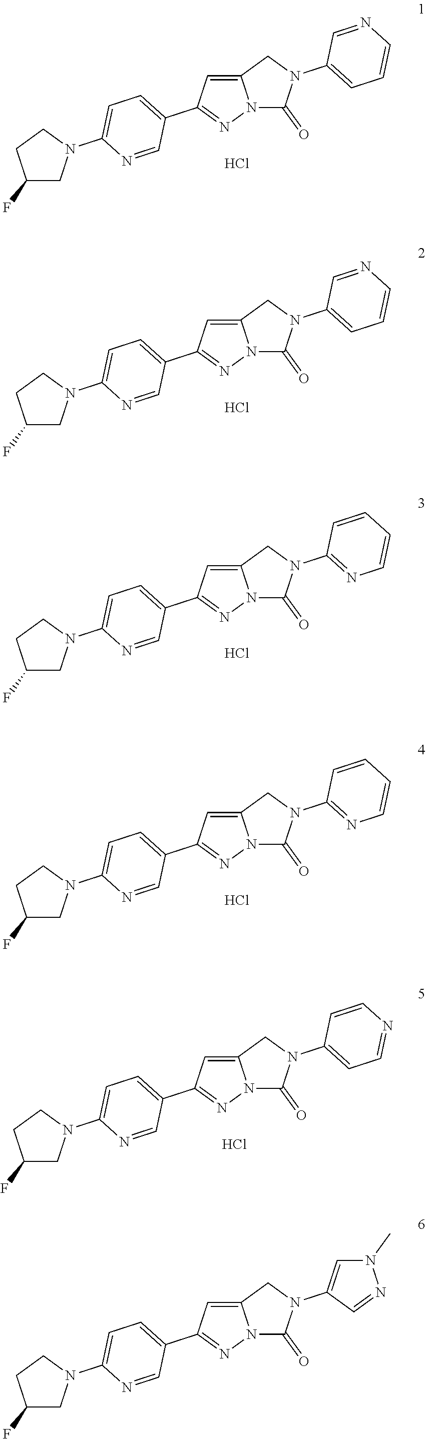 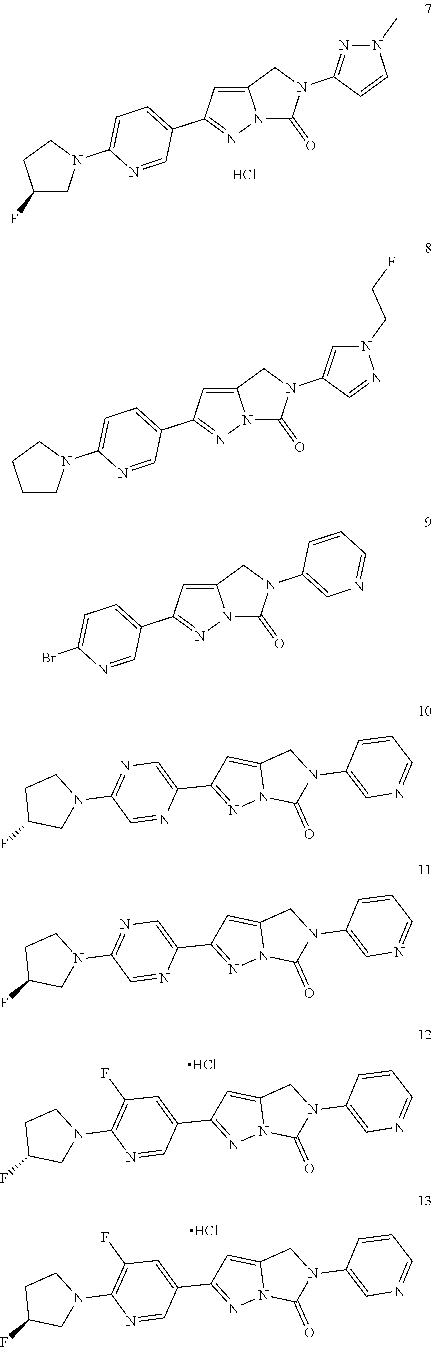 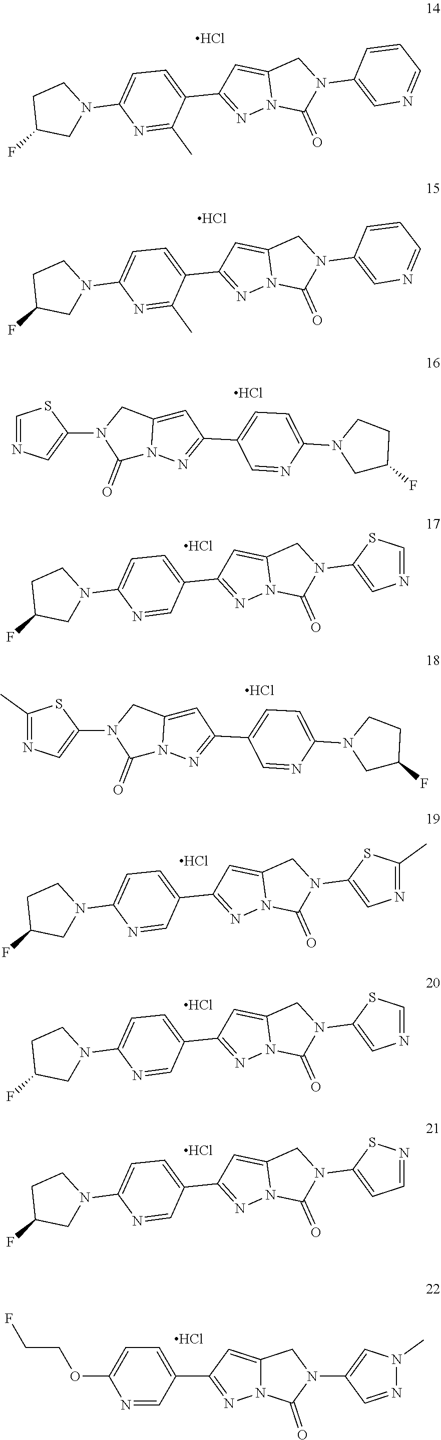 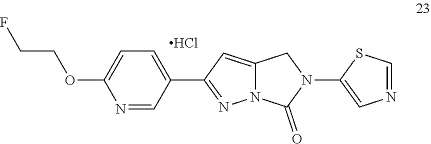 [SMILES: O=C1N(c2cncs2)Cc2cc(-c3ccc(OCCF)nc3)nn21 ; Cl]
or a detectably labelled compound, stereoisomer, racemic mixture, pharmaceutically acceptable salt, hydrate, or solvate thereof.

9. The compound according to claim 1, wherein the compound is a detectably labelled compound; wherein the detectable label is preferably selected from a radioisotope, preferably ²H, ³H or ¹⁸F.

10. (canceled)

11. The compound according to claim 9 wherein R¹ is 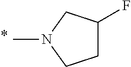
and the compound of formula (I) is detectably labelled at least at one available position by ³H.

12. A diagnostic composition comprising a compound according to claim 1, and at least one pharmaceutically acceptable excipient, carrier, diluent and/or adjuvant.

13. A method for imaging of alpha-synuclein aggregates including, but not limited to, Lewy bodies and/or Lewy neurites or positron emission tomography imaging of alpha-synuclein aggregates including, but not limited to, Lewy bodies and/or Lewy neurites, comprising employing the compound according to claim 9.

14. (canceled)

15. The method according to claim 13, wherein the imaging comprises in vitro imaging, ex vivo imaging, or in vivo imaging, preferably in vivo imaging, more preferably brain imaging.

16. A method for diagnostics, comprising employing the compound according to claim 9, wherein the diagnostics are the diagnostics of a disease, disorder or abnormality associated with alpha-synuclein aggregates including, but not limited to, Lewy bodies and/or Lewy neurites or a predisposition therefor, wherein the disease, disorder or abnormality is optionally selected from Parkinson's disease (including sporadic, familial with alpha-synuclein mutations, familial with mutations other than alpha-synuclein, pure autonomic failure or Lewy body dysphagia), SNCA duplication carrier, Lewy Body dementia (LBD), dementia with Lewy bodies (DLB) (including “pure” Lewy body dementia), Parkinson's disease dementia (PDD), diffuse Lewy body disease (DLBD), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease, Down syndrome, multiple system atrophy (MSA) (including Shy-Drager syndrome, striatonigral degeneration or olivopontocerebellar atrophy), traumatic brain injury, chronic traumatic encephalopathy, dementia puglistica, tauopathies (including Pick's disease, frontotemporal dementia, progressive supranuclear palsy, corticobasal degeneration, Niemann-Pick type C1 disease, frontotemporal dementia with Parkinsonism linked to chromosome 17), Creutzfeldt-Jakob disease, Huntington's disease, motor neuron disease, amyotrophic lateral sclerosis (including sporadic, familial or ALS-dementia complex of Guam), neuroaxonal dystrophy, neurodegeneration with brain iron accumulation type 1 (including Hallervorden-Spatz syndrome), prion diseases, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder.

17. (canceled)

18. The method according to claim 16, wherein the disease is Parkinson's disease, multiple system atrophy, dementia with Lewy bodies, Parkinson's disease dementia, SNCA duplication carrier, or Alzheimer's disease.

19. (canceled)

20. (canceled)

21. (canceled)

22. (canceled)

23. (canceled)

24. (canceled)

25. (canceled)

26. (canceled)

27. (canceled)

28. (canceled)

29. A method comprising the steps:
(a) Bringing a sample or a specific body part or body area suspected to contain an alpha-synuclein aggregates, including but not limited to, Lewy bodies and/or Lewy neurites, into contact with a compound according to claim 1;
(b) Allowing the compound to bind to the alpha-synuclein aggregates, including but not limited to, Lewy bodies and/or Lewy neurites;
(c) Detecting the compound bound to the alpha-synuclein aggregates, including but not limited to, Lewy bodies and/or Lewy neurites using positron emission tomography; and
wherein the method is selected from:
(i) a method for the detection and optionally quantification of alpha-synuclein aggregates, including but not limited to, Lewy bodies and/or Lewy neurites, in a tissue of a subject,
(ii) a method of collecting data for the diagnosis of a disease, disorder or abnormality associated with alpha-synuclein aggregates including, but not limited to, Lewy bodies and/or Lewy neurites,
(iii) a method of collecting data for determining a predisposition to a disease, disorder or abnormality associated with alpha-synuclein aggregates including, but not limited to, Lewy bodies and/or Lewy neurites,
(iv) a method of collecting data for prognosing a disease, disorder or abnormality associated with alpha-synuclein aggregates including, but not limited to, Lewy bodies and/or Lewy neurites,
(v) a method of collecting data for monitoring the progression of a disease, disorder or abnormality associated with alpha-synuclein aggregates including, but not limited to, Lewy bodies and/or Lewy neurites in a patient, or
(vi) a method of collecting data for predicting responsiveness of a patient suffering from a disease, disorder or abnormality associated with alpha-synuclein aggregates including, but not limited to, Lewy bodies and/or Lewy neurites to a treatment with a medicament.

30. (canceled)

31. (canceled)

32. (canceled)

33. (canceled)

34. (canceled)

35. (canceled)

36. A compound of formula (III-F) or (III-F′) 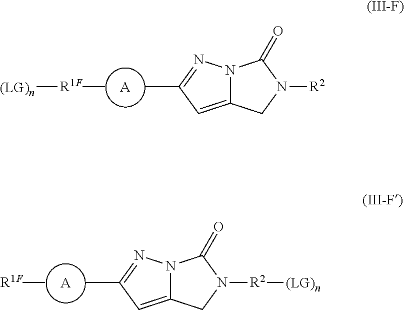
or a stereoisomer, racemic mixture, pharmaceutically acceptable salt, hydrate, or solvate thereof, wherein 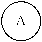
is a 6-membered heteroaryl; which is optionally substituted with at least one substituent independently selected from halo, or C₁-C₄alkyl
R^{1F} is a 4- to 6-membered heterocyclyl or C₁-C₄alkoxy;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
LG is a leaving group, which is preferably selected from bromo, chloro, iodo, C₁-C₄alkylsulfonate and C₆-C₁₀arylsulfonate, wherein the C₆-C₁₀arylsulfonate can be optionally substituted with —CH₃ or —NO₂; and
n is at least 1.

37. (canceled)

38. A compound of formula (III-H) 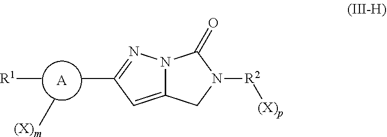
or a stereoisomer, racemic mixture, pharmaceutically acceptable salt, hydrate, or solvate thereof, wherein 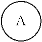
is a 6-membered heteroaryl which is optionally substituted with at least one substituent independently selected from halo, or C₁-C₄alkyl;
R¹ is halo or a 4- to 6-membered heterocyclyl which is optionally substituted with at least one halo, or haloC₁-C₄alkoxy;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
m is 0, 1, or 2;
p is 0, 1, or 2; and
X is bromo, chloro or iodo;
with the proviso that the compound of formula (III-H) comprises at least one X.

39. A method of preparing the compound according to claim 9 comprising reacting the compound of formula (III-F) or (III-F′) according to claim 36 with a ¹⁸F-fluorinating agent, so that LG is replaced by ¹⁸F, wherein the ¹⁸F-fluorinating agent is preferably selected from K¹⁸F, Rb¹⁸F, Cs¹⁸F, Na¹⁸F, Kryptofix[222]K¹⁸F, tetra(C₁₋₆alkyl)ammonium salt of ¹⁸F, and tetrabutylammonium [¹⁸F]fluoride.

40. (canceled)

41. A method of preparing the compound according to claim 9, comprising reacting the compound of formula (III-H) according to claim 38 with a ³H radiolabelling agent.

42. A method of screening comprising employing the compound according to claim 1, wherein the compound is employed as an in vitro analytical reference or an in vitro screening tool.

43. A test kit for the detection and/or diagnosis of a disease, disorder or abnormality associated with alpha-synuclein aggregates, wherein the test kit comprises at least one compound as defined in claim 1.

44. A kit for preparing a radiopharmaceutical preparation, wherein the kit comprises a sealed vial containing at least one compound as defined in claim 36.
